(19) United States
(12) Patent Application Publication
(10) Pub. No.: US 2024/0415818 A1
(43) Pub. Date: Dec. 19, 2024

(54) COMBINATION THERAPY FOR THE TREATMENT OF GASTROINTESTINAL STROMAL TUMORS

(71) Applicant: Deciphera Pharmaceuticals, LLC, Waltham, MA (US)

(72) Inventors: Daniel L. Flynn, Waltham, MA (US); Bryan D. Smith, Waltham, MA (US); Anu Gupta, Waltham, MA (US)

(21) Appl. No.: 18/631,891

(22) Filed: Apr. 10, 2024

Related U.S. Application Data
(62) Division of application No. 16/943,821, filed on Jul. 30, 2020, now Pat. No. 11,986,463.
Application No. 16/943,821 is a continuation of application No. PCT/US2019/016148, filed on Jan. 31, 2019.
(60) Provisional application No. 62/624,448, filed on Jan. 31, 2018.

(51) Int. Cl. A61K 31/4375; A61K 45/06; A61P 35/04

(52) U.S. Cl. CPC A61K 31/4375; A61K 45/06; A61P 35/04

(57) ABSTRACT

The present disclosure relates to the use of 1-[4-bromo-5-[1-ethyl-7-(methylamino)-2-oxo-1,2-dihydro-1,6-naphthyridin-3-yl]-2-fluorophenyl]-3-phenylurea or 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof, in combination with a MAPKAP kinase inhibitor for the treatment of cancers, including c-KIT-mediated cancers, such as GIST.

### CROSS-REFERENCE TO RELATED APPLICATIONS

This application is divisional of U.S. application Ser. No. 16/943,821 filed Jul. 30, 2020, which is a continuation of International Application Number PCT/US2019/016148 filed Jan. 31, 2019, which claims priority to U.S. Ser. No. 62/624,448 filed Jan. 31, 2018, the contents of which are incorporated herein by reference in their entirety.

### BACKGROUND

c-KIT (also known as KIT, CD117, and stem cell factor receptor) is a 145 kDa transmembrane tyrosine kinase protein that acts as a type-III receptor. The c-KIT proto-oncogene, located on chromosome 4q11-21, encodes the c-KIT receptor, whose ligand is the stem cell factor (SCF, steel factor, kit ligand, mast cell growth factor). The receptor has tyrosine-protein kinase activity, and binding of the ligand SCF leads to the autophosphorylation of c-KIT and its association with substrates such as phosphatidylinositol 3-kinase (PI3K). Tyrosine phosphorylation by protein tyrosine kinases is of particular importance in cellular signaling and can mediate signals for major cellular processes, such as proliferation, survival, differentiation, apoptosis, attachment, invasiveness and migration.

The role of c-KIT expression and activity has been studied in hematologic and solid tumors, such as acute leukemias and gastrointestinal stromal tumors (GISTs). Most GISTs have primary activating mutations in the genes encoding the closely related RTKs c-KIT (75-80% of GIST) or PDGFRα (8% of the non-c-KIT mutated GIST), and gain-of-function mutations of the c-KIT gene and the expression of constitutively phosphorylated c-KIT are found in many GIST. The majority of primary GIST-causing c-KIT mutations affect the juxtamembrane (JM) region of the protein encoded by exon 11 and consist of in-frame deletions or insertions, or missense mutations (i.e., V560D). c-KIT exon 11 mutations have been identified as primary mutations in approximately 65% of GISTs. Such JM domain mutations disrupt the autoinhibition mechanism of c-KIT kinase, leading to constitutive kinase activity and cell-transforming events causative of GIST. Other primary GIST-causing c-KIT mutations are located in exon 9 (AY501-502 duplication/insertion, 8%), exon 13(mutation, 1%), and exon 17 (mutation, 1%).

The clinical importance of c-KIT expression in malignant tumors was demonstrated in studies with Gleevec® (imatinib mesylate, STI571 (signal transduction inhibitor number 571), Novartis Pharma AG Basel, Switzerland), which specifically inhibits tyrosine kinase receptors. Moreover, a clinically relevant breakthrough has been the finding of anti-tumor effects of this compound in GIST, a group of tumors regarded as being generally resistant to conventional chemotherapy. However, while major responses were seen after first-line treatment of GIST with Gleevec®, an inhibitor of c-KIT, and a substantial number of patients with metastatic and/or inoperable GIST benefit from treatment with Gleevec®, complete tumor remissions are rare, and about 50% of patients experience disease recurrence within two years of treatment. It has also been reported that a combination of the c-KIT inhibitor imatinib and the MEK kinase inhibitor MEK162 resulted in an increased growth suppression in vitro and tumor regression in vivo in various GIST cancer cell lines compared to treatment with either single agent.

GIST most often become Gleevec® resistant, and molecularly targeted small molecule therapies that target c-KIT secondary mutations remain elusive. GIST patients who relapse after treatment with Gleevec® or Sutent® have disease still driven by c-KIT mutations. These secondary mutations occur on the same alleles as the primary JM-region mutation, and thus represent even more aggressive activated forms of c-KIT than the original primary mutation. These secondary mutants of c-KIT identified in GIST lead to acquired drug resistance. Secondary mutations are found in the ATP binding pocket (exon 13, i.e. K642E, V654A; exon 14, i.e. T6701), and activation loop (exon 17, i.e. N822K, D816H, D816V, D820A; exon 18 A829P). These various secondary c-KIT mutations have been reported: . Sunitinib malate (Sutent™, Pfizer) is an inhibitor of multiple RTKs, notably in this context, c-KIT and PDGFRα, and has been shown to be effective against certain imatinib-resistant c-KIT mutants, such as the ATP-binding pocket mutants V654A and T670I. Certain Gleevec®-resistant mutants are also resistant to sunitinib, such as D816H and D816V which are located in the activation loop of the c-KIT catalytic domain encoded by exon 17. Median survival after progression due to Gleevec®-resistance remains relatively short.

It has been demonstrated that complex, multiple secondary c-KIT mutations can arise and vary within individual patients, such variation in mutational status of c-KIT being demonstrated by biopsy samples obtained from different progressing metastases within each patient. The complex c-KIT mutational heterogeneity within individual patients underscores an unmet medical need to identify inhibitors of c-KIT kinase that are effective across a broad spectrum of c-KIT primary and secondary mutations. In addition, there is a need to identify therapies that are cytotoxic, or cytocidal, to c-KIT-mediated GISTs, as opposed to merely being cytostatic, and which result in disease remission and/or reduced disease recurrence.

### SUMMARY

The instant disclosure is drawn to the combination of the c-KIT inhibitor Compound A or the c-KIT inhibitor Compund B with an inhibitor of the MAPKAP kinase signaling pathway. Herein, the MAPKAP pathway is defined as the signaling through the kinases RAF→MEK→ERK. It has been unexpectedly demonstrated that combination of the c-KIT inhibitor Compound A or Compound B with a MEK inhibitor, including trametinib, an ERK inhibitor including ulixertinib, or a RAF inhibitor including LY3009120 leads to cell death, apoptosis, or prolonged cell stasis of GIST cells, enhanced GIST tumor regression in vivo, or eradication of GIST cells in colony formation assays as compared to a combination of imatinib with a MEK inhibitor or to single agent treatment with Compound A, imatinib, or a MEK inhibitor. Additionally it has been demonstrated that combination of the c-KIT inhibitor Compound A with a MEK inhibitor leads to enhanced cell death or apoptosis and eradication of GIST cancer cell lines that are resistant to imatinib in combination with a MEK inhibitor. In colony outgrowth studies in GIST cells, Compound A exhibited superior synergy in combination with a MEK inhibitor compared to imatinib in combination with a MEK inhibitor. This disclosure, in part, relates to methods of treating tumors in patients using Compound A as described herein or a pharmaceutically acceptable salt thereof.

For example, described herein is a method of treating a tumor having one or more c-KIT mutations in a patient in need thereof, comprising administering to the patient: an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and an effective amount of a mitogen-activated protein kinase inhibitor (MEK inhibitor) and/or an effective amount of an extracellular signal regulated kinase inhibitor (ERK inhibitor).

This disclosure also provides a method of treating a solid tumor in an imatinib resistant patient, comprising: administering to the patient an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and administering to the patient an effective amount of a MEK or ERK inhibitor selected from the group consisting of trametinib, binimetinib, cobimetinib, and ulixertinib wherein the solid tumor is selected from the group consisting of of lung adenocarcinoma, squamous cell lung cancer, glioblastoma, pediatric glioma, astrocytoma, sarcoma, gastrointestinal stromal tumor (GIST), and melanoma.

A method of treating an imatinib resistant gastrointestinal stromal tumor or imatinib resistant melanoma in a patient in need thereof is also contemplated herein, comprising administering to the patient an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and administering to the patient an effective amount of a MEK or ERK inhibitor selected from the group consisting of trametinib, binimetinib, cobimetinib, and ulixertinib.

This disclosure also provides a method of treating a solid tumor in a patient in need thereof, comprising: administering to the patient an effective amount of 1-[4-bromo-5-[1-ethyl-7-(methylamino)-2-oxo-1,2-dihydro-1,6-naphthyridin-3-yl]-2- fluorophenyl]-3-phenylurea, or a pharmaceutically acceptable salt thereof; and administering to the patient an effective amount of a MEK or ERK inhibitor selected from the group consisting of trametinib, binimetinib, cobimetinib, and ulixertinib wherein the solid tumor is selected from the group consisting of of lung adenocarcinoma, squamous cell lung cancer, glioblastoma, pediatric glioma, astrocytoma, sarcoma, gastrointestinal stromal tumor (GIST), and melanoma.

A method of treating an gastrointestinal stromal tumor or melanoma in a patient in need thereof is also contemplated herein, comprising administering to the patient an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and administering to the patient an effective amount of a MEK or ERK inhibitor selected from the group consisting of trametinib, binimetinib, cobimetinib, and ulixertinib.

Additionally contemplated herein is a method of treating a solid tumor in a patient need thereof, comprising administering to the patient an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and administering to the patient an effective amount of a RAF inhibitor.

### BRIEF DESCRIPTION OF THE DRAWINGS

FIG. 1A shows a graphical representation of Caspase activity following various treatments with Compound A and trametinib for 24 hours in GIST-T1 cells.

FIG. 1B provides a synergy matrix chart based on the combination index method for various treatments with Compound A and trametinib for 24 hours in GIST-T1 cells and a Combination Index Plot demonstrating synergy graphed as combination index (CI) on the y-axis and Fraction affected (Fa) on the x-axis.

FIG. 1C shows a graphical representation of Caspase activity following various treatments with Compound A and trametinib for 48 hours in GIST-T1 cells.

FIG. 1D provides a synergy matrix chart based on the combination index method for various treatments with Compound A and trametinib for 48 hours in GIST-T1 cells and a Combination Index Plot demonstrating synergy graphed as combination index (CI) on the y-axis and Fraction affected (Fa) on the x-axis.

FIG. 1E shows a graphical representation of Caspase activity following various treatments with Compound A and trametinib for 24 hours in GIST-T1/D816E imatinib resistant cells.

FIG. 1F provides a synergy matrix chart based on the combination index method for various treatments with Compound A and trametinib for 24 hours in GIST-T1/D816E imatinib resistant cells and a Combination Index Plot demonstrating synergy graphed as combination index (CI) on the y-axis and Fraction affected (Fa) on the x-axis.

FIG. 1G shows a graphical representation of Caspase activity following various treatments with Compound A and trametinib for 24 hours in GIST-T1/T670I imatinib resistant cells.

FIG. 1H provides a synergy matrix chart based on the combination index method for various treatments with Compound A and trametinib for 24 hours in GIST-T1/T670I imatinib resistant cells and a Combination Index Plot demonstrating synergy graphed as combination index (CI) on the y-axis and Fraction affected (Fa) on the x-axis.

FIG. 2A shows a graphical representation of Caspase activity following various treatments with Compound B and trametinib for 24 hours in GIST-T1 cells.

FIG. 2B provides a synergy matrix chart based on the combination index method for various treatments with Compound B and trametinib for 24 hours in GIST-T1 cells and a Combination Index Plot demonstrating synergy graphed as combination index (CI) on the y-axis and Fraction affected (Fa) on the x-axis.

FIG. 2C shows a graphical representation of Caspase activity following various treatments with Compound B and trametinib for 24 hours in GIST-T1/D816E imatinib resistant cells.

FIG. 2D provides a synergy matrix chart based on the combination index method for various treatments with Compound B and trametinib for 24 hours in GIST-T1/D816E imatinib resistant cells and a Combination Index Plot demonstrating synergy graphed as combination index (CI) on the y-axis and Fraction affected (Fa) on the x-axis.

FIG. 2E shows a graphical representation of Caspase activity following various treatments with Compound B and trametinib for 24 hours in GIST-T1/T670I imatinib resistant cells.

FIG. 2F provides a synergy matrix chart based on the combination index method for various treatments with Compound B and trametinib for 24 hours in GIST-T1/T670I imatinib resistant cells and a Combination Index Plot demonstrating synergy graphed as combination index (CI) on the y-axis and Fraction affected (Fa) on the x-axis.

FIG. 3A shows a graphical representation of Caspase activity following various treatments with Compound A and binimetinib for 24 hours in GIST-T1 cells.

FIG. 3B provides a synergy matrix chart based on the combination index method for various treatments with Compound A and binimetinib for 24 hours of GIST-T1 cells and a Combination Index Plot demonstrating synergy graphed as combination index (CI) on the y-axis and Fraction affected (Fa) on the x-axis.

FIG. 3C shows a graphical representation of Caspase activity following various treatments with Compound A and binimetinib for 24 hours in GIST-T1/D816E imatinib resistant cells.

FIG. 3D provides a synergy matrix chart based on the combination index method for various treatments with Compound A and binimetinib for 24 hours in GIST-T1/D816E imatinib resistant cells and a Combination Index Plot demonstrating synergy graphed as combination index (CI) on the y-axis and Fraction affected (Fa) on the x-axis.

FIG. 3E shows a graphical representation of Caspase activity following various treatments with Compound A and binimetinib for 24 hours in GIST-T1/T670I imatinib resistant cells.

FIG. 3F provides a synergy matrix chart based on the combination index method for various treatments with Compound A and binimetinib for 24 hours in GIST-T1/T670I imatinib resistant cells and a Combination Index Plot demonstrating synergy graphed as combination index (CI) on the y-axis and Fraction affected (Fa) on the x-axis.

FIG. 4A shows a graphical representation of Caspase activity following various treatments with Compound B and binimetinib for 24 hours in GIST-T1 cells.

FIG. 4B provides a synergy matrix chart based on the combination index method for various treatments with Compound B and binimetinib for 24 hours in GIST-T1 cells and a Combination Index Plot demonstrating synergy graphed as combination index (CI) on the y-axis and Fraction affected (Fa) on the x-axis.

FIG. 4C shows a graphical representation of Caspase activity following various treatments with Compound B and binimetinib for 24 hours in GIST-T1/D816E imatinib resistant cells.

FIG. 4D provides a synergy matrix chart based on the combination index method for various treatments with Compound B and binimetinib for 24 hours in GIST-T1/D816E imatinib resistant cells and a Combination Index Plot demonstrating synergy graphed as combination index (CI) on the y-axis and Fraction affected (Fa) on the x-axis.

FIG. 4E shows a graphical representation of Caspase activity following various treatments with Compound B and binimetinib for 24 hours in GIST-T1/T670I imatinib resistant cells.

FIG. 4F provides a synergy matrix chart based on the combination index method for various treatments with Compound B and binimetinib for 24 hours in GIST-T1/T670I imatinib resistant cells and a Combination Index Plot demonstrating synergy graphed as combination index (CI) on the y-axis and Fraction affected (Fa) on the x-axis.

FIG. 5A shows a graphical representation of Caspase activity following various treatments with Compound A and cobimetinib for 24 hours in GIST-T1 cells.

FIG. 5B provides a synergy matrix chart based on the combination index method for various treatments with Compound A and cobimetinib for 24 hours in GIST-T1 cells and a Combination Index Plot demonstrating synergy graphed as combination index (CI) on the y-axis and Fraction affected (Fa) on the x-axis.

FIG. 5C shows a graphical representation of Caspase activity following various treatments with Compound A and cobimetinib for 24 hours in GIST-T1/D816E imatinib resistant cells.

FIG. 5D provides a synergy matrix chart based on the combination index method for various treatments with Compound A and cobimetinib for 24 hours in GIST-T1/D816E imatinib resistant cells and a Combination Index Plot demonstrating synergy graphed as combination index (CI) on the y-axis and Fraction affected (Fa) on the x-axis.

FIG. 5E shows a graphical representation of Caspase activity following various treatments with Compound A and cobimetinib for 24 hours in GIST-T1/T670I imatinib resistant cells.

FIG. 5F provides a synergy matrix chart based on the combination index method for various treatments with Compound A and cobimetinib for 24 hours in GIST-T1/T670I imatinib resistant cells and a Combination Index Plot demonstrating synergy graphed as combination index (CI) on the y-axis and Fraction affected (Fa) on the x-axis.

FIG. 6A shows a graphical representation of Caspase activity following various treatments with Compound B and cobimetinib for 24 hours in GIST-T1 cells.

FIG. 6B provides a synergy matrix chart based on the combination index method for various treatments with Compound B and cobimetinib for 24 hours of GIST-T1 cells and a Combination Index Plot demonstrating synergy graphed as combination index (CI) on the y-axis and Fraction affected (Fa) on the x-axis.

FIG. 6C shows a graphical representation of Caspase activity following various treatments with Compound B and cobimetinib for 24 hours in GIST-T1/D816E imatinib resistant cells.

FIG. 6D provides a synergy matrix chart based on the combination index method for various treatments with Compound B and cobimetinib for 24 hours in GIST-T1/D816E imatinib resistant cells and a Combination Index Plot demonstrating synergy graphed as combination index (CI) on the y-axis and Fraction affected (Fa) on the x-axis.

FIG. 6E shows a graphical representation of Caspase activity following various treatments with Compound B and cobimetinib for 24 hours in GIST-T1/T670I imatinib resistant cells.

FIG. 6F provides a synergy matrix chart based on the combination index method for various treatments with Compound B and cobimetinib for 24 hours in GIST-T1/T670I imatinib resistant cells and a Combination Index Plot demonstrating synergy graphed as combination index (CI) on the y-axis and Fraction affected (Fa) on the x-axis.

FIG. 7A shows a graphical representation of Caspase activity following various treatments with Compound A and the ERK inhibitor ulixertinib for 24 hours in GIST-T1 cells.

FIG. 7B provides a synergy matrix chart based on the combination index method for various treatments with Compound A and ulixertinib for 24 hours in GIST-T 1 cells and a Combination Index Plot demonstrating synergy graphed as combination index (CI) on the y-axis and Fraction affected (Fa) on the x-axis.

FIG. 7C shows a graphical representation of Caspase activity following various treatments with Compound A and ulixertinib for 24 hours in GIST-T1/T670I imatinib resistant cells.

FIG. 7D provides a synergy matrix chart based on the combination index method for various treatments with Compound A and ulixertinib for 24 hours in GIST-T1/T670I imatinib resistant cells

FIG. 8A shows images of representative culture plates and a graphical representation of the number of GIST-T1 colonies counted following various treatments with Compound A, imatinib, and trametinib for 2 weeks followed by a 9 day recovery period.

FIG. 8B shows images of representative culture plates and a graphical representation of the number of GIST-T1/D816E colonies counted following various treatments with Compound A, imatinib, and trametinib for 2 weeks followed by a 10 day recovery period. The right upper panel shows representative culture plates after additional 10 days of recovery.

FIG. 8C shows images of representative culture plates and a graphical representation of the number of GIST-T1/D816E colonies counted following various treatments with Compound A, imatinib (IM), and trametinib for 2 weeks followed by a 10 day recovery period.

FIG. 8D shows images of representative culture plates and a graphical representation of the number of GIST-T1/T670I colonies counted following various treatments with Compound A, imatinib, and trametinib for 2 weeks followed by a 10 day recovery period.

FIG. 9A shows images of representative culture plates and a graphical representation of the number of GIST-T1 colonies counted following various treatments with Compound B and trametinib for 2 weeks followed by a 10 day recovery period.

FIG. 9B shows images of representative culture plates and a graphical representation of the number of GIST-T1/D816E colonies counted following various treatments with Compound B and trametinib for 2 weeks followed by a 10 day recovery period. The right upper panel shows representative culture plates after additional 10 days of recovery.

FIG. 9C shows images of representative culture plates showing the number of GIST-T1/T670I colonies counted following various treatments with Compound B and trametinib for 2 weeks followed by a 10 day recovery period.

FIG. 10A shows images of representative culture plates and a graphical representation of the number of GIST-T1 colonies counted following various treatments with Compound A, imatinib, and binimetinib for 2 weeks followed by a 10 day recovery period. The right upper panel shows representative culture plates after additional 10 days of recovery.

FIG. 10B shows images of representative culture plates and a graphical representation of the number of GIST-T1/D816E colonies counted following various treatments with Compound A, imatinib, and binimetinib for 2 weeks followed by a 10 day recovery period. The right upper panel shows representative culture plates after additional 10 days of recovery.

FIG. 10C shows images of representative culture plates and a graphical representation of the number of GIST-T1/T670I colonies counted following various treatments with Compound A, imatinib, and binimetinib for 2 weeks followed by a 10 day recovery period. The right upper panel shows representative culture plates after additional 10 days of recovery.

FIG. 11A shows images of representative culture plates and a graphical representation of the number of GIST-T1 colonies counted following various treatments with Compound B and binimetinib for 2 weeks followed by a 10 day recovery period. The right upper panel shows representative culture plates after additional 10 days of recovery.

FIG. 11B shows images of representative culture plates and a graphical representation of the number of GIST-T1/D816E colonies counted following various treatments with Compound B and binimetinib for 2 weeks followed by a 10 day recovery period. The right upper panel shows representative culture plates after additional 10 days of recovery.

FIG. 11C shows images of representative culture plates showing the number of GIST-T1/D816E colonies counted following various treatments with Compound B and binimetinib for 2 weeks followed by a 10 day recovery period. The right upper panel shows representative culture plates after additional 10 days of recovery.

FIG. 12A shows images of representative culture plates and a graphical representation of the number of GIST-T1 colonies counted following various treatments with Compound A, imatinib, and cobimetinib for 2 weeks followed by a 10 day recovery period. The right upper panel shows representative culture plates after additional 10 days of recovery.

FIG. 12B shows images of representative culture plates and a graphical representation of the number of GIST-T1/D816E colonies counted following various treatments with Compound A, imatinib, and cobimetinib for 2 weeks followed by a 10 day recovery period. The right upper panel shows representative culture plates after additional 10 days of recovery.

FIG. 12C shows images of representative culture plates and a graphical representation of the number of GIST-T1/T670I colonies counted following various treatments with Compound A, imatinib, and cobimetinib for 2 weeks followed by a 10 day recovery period. The right upper panel shows representative culture plates after additional 10 days of recovery.

FIG. 13A shows images of representative culture plates and a graphical representation of the number of GIST-T1 colonies counted following various treatments with Compound B and cobimetinib for 2 weeks followed by a 10 day recovery period. The right upper panel shows representative culture plates after additional 10 days of recovery.

FIG. 13B shows images of representative culture plates and a graphical representation of the number of GIST-T1/D816E colonies counted following various treatments with Compound B and cobimetinib for 2 weeks followed by a 10 day recovery period. The right upper panel shows representative culture plates after additional 10 days of recovery.

FIG. 13C shows images of representative culture plates showing the number of GIST-T1/T670I colonies counted following various treatments with Compound B and cobimetinib for 2 weeks followed by a 10 day recovery period. The right upper panel shows representative culture plates after additional 10 days of recovery.

FIG. 14A shows images of representative culture plates and a graphical representation of the number of GIST-T1 colonies counted following various treatments with Compound A and the ERK inhibitor ulixertinib for 2 weeks followed by a 10 day recovery period.

FIG. 14B shows images of representative culture plates and a graphical representation of the number of GIST-T1/D816E colonies counted following various treatments with Compound A and ulixertinib for 2 weeks followed by a 10 day recovery period.

FIG. 14C shows images of representative culture plates and a graphical representation of the number of GIST-T1/T670I colonies counted following various treatments with Compound A and ulixertinib for 2 weeks followed by a 10 day recovery period. The right upper panel shows representative culture plates after additional 10 days of recovery.

FIG. 15 shows images of representative culture plates and a graphical representation of the number of GIST-T1/D816E colonies counted following various treatments with Compound B and ulixertinib for 2 weeks.

FIG. 16A shows graphical representations of Caspase activity from various treatments with Compound A and trametinib for 48 hours in vector control or N-ras G12D transfected GIST-T1 cells.

FIG. 16B and FIG. 16C show images of representative culture plates of vector control (FIG. 16B) or N-ras G12D (FIG. 16C) transfected GIST-T1 colonies following various treatments with Compound A, imatinib, and trametinib and a subsequent 10 day recovery period.

FIG. 16D and FIG. 16E show a graphical representation of the number of vector control (FIG. 15D) or N-ras G12D (FIG. 16E) transfected GIST-T1 colonies counted following various treatments treatments with Compound A, imatinib, and trametinib and a subsequent 10 day recovery period.

FIG. 16F shows images of representative culture plates of N-ras G12D transfected GIST-T1 colonies following various treatments with Compound A and trametinib and a subsequent extended 21 day recovery period.

FIG. 17A provides graphical representations of Ba/F3 V560D KIT cell outgrowth of c-KIT secondary mutations in T670I, K807E, or D816V, or cell outgrowth retaining only the original c-KIT V560D mutation plus additional non-c-KIT resistance mechanisms upon saturation mutagenesis followed by single agent treatments with either imatinib (left panel) or Compound A (right panel).

FIG. 17B provides graphical representations of Ba/F3 V560D KIT cell outgrowth of c-KIT secondary mutations in T670I, K807E, or D816V, or cell outgrowth retaining only the original c-KIT V560D mutation plus additional non-c-KIT resistance mechanisms upon saturation mutagenesis followed by combination treatments with either imatinib plus trametinib (left panel) or Compound A plus trametinib (right panel).

FIG. 18A provides a graphical representation of GIST T1 xenograft tumor growth following treatments with single agent Compound A, single agent trametinib, or with a combination of Compound A and trametinib.

FIG. 18B is a blow up of the graphical representation from FIG. 18A, showing resolution of effects on tumor regression following treatments with single agent Compound A, single agent trametinib, or with a combination of Compound A and trametinib.

### DETAILED DESCRIPTION

It has been found that the combination of 1-[4-bromo-5-[1-ethyl-7-(methylamino)-2-oxo-1,2-dihydro-1,6-naphthyridin-3-yl]-2-fluorophenyl]-3-phenylurea (Compound A) and a MAPKAP kinase pathway inhibitor, e.g., trametinib, unexpectedly synergizes to lead to cell death, apoptosis, or prolonged cell stasis of GIST cells, to induce eradication of tumor cells, to induce tumor regression, to reduce tumor volume, to inhibit tumor regrowth, and/or to lead to enhanced cell death, apoptosis, cell stasis or eradication of GIST cancer cell lines that are resistant to imatinib in combination with a MEK inhibitor in the accompanying Examples. In addition, the combination therapy methods disclosed herein appear to be cytocidal as opposed to merely cytostatic.

Without wishing to be bound to any particular theory, it is believed that many c-KIT inhibitors only inhibit certain mutant forms of c-KIT, such as the prominent exon 11 mutation observed in GIST. Other mutant forms of c-KIT appear resistant to many c-KIT inhibitors, and these often arise as secondary mutations in exons 13, 14, 17 and 18 that render a tumor resistant to treatment with c-KIT inhibitors. The present disclosure provides methods of treating tumors, e.g., c-KIT-mediated tumors such as GIST, by inhibiting both c-KIT and a MAPKAP pathway kinase using a c-KIT inhibitor disclosed herein as Compound A or a pharmaceutically acceptable salt thereof or Compound B or a pharmaceutically acceptable salt thereof. Surprisingly, Compound A and Compound B (and pharmaceutically acceptable salts thereof) synergize with a MEK inhibitor, a ERK inhibitor, or a RAF inhibitor to induce cell death, apoptosis, or prolonged cell stasis of GIST cells, to induce eradication of tumor cells to the limit of detection, to reduce tumor volume, to inhibit tumor regrowth and/or to lead to enhanced cell death, apoptosis, cell stasis, or eradication to the limit of detection, of GIST cancer cell lines that are resistant to imatinib in combination with a MAPKAP kinase inhibitor. Compound A exhibits superior potency and synergy in combination with MEK inhibition compared to imatinib in combination with MEK inhibition in GIST cells containing c-KIT resistance mutations. Further, the level of potency and synergy and the degree of GIST tumor cell prolonged cell stasis or eradication of Compound A in combination with MEK inhibition is superior to that of imatinib in combination with MEK inhibition even in cell lines known to be sensitive to imatinib. Again without wishing to be bound to any particular theory, it is believed that Compound A is able to inhibit a wider range of mutant forms of c-KIT than previous c-KIT inhibitors, including imatinib, in GIST cells possibly through mechanisms that include the inhibition of drug efflux pumps, including the BCRP efflux pump, within tumor cells, e.g., GIST cells. Imatinib is a substrate for the BCRP efflux pump, leading to lower intracellular concentrations in tumor cells where this efflux pump is present (Eechoute, K, et al, Clin Cancer Res. 2015, 17, 406-15). GIST tumors have been demonstrated to have overexpression of the BCRP efflux pump in 93% (42/45) of GIST patient tumors evaluated (Feldman, R, et al. J Clin Oncol. 2015, 33, 58). Compound A is a potent inhibitor of the BCRP efflux transporter, exhibiting an IC₅₀ value of 40 nM.

Accordingly, in certain embodiments, the present disclosure provides methods for inducing prolonged tumor cell stasis, inducing cell death, inducing apoptosis of tumor cells, inducing eradication of tumor cells, inducing tumor regression, reducing tumor volume, inhibiting tumor regrowth, or inhibiting the growth of resistant tumor cells, the methods comprising administering to a patient in need thereof an effective amount of: (i) 1-[4-bromo-5-[1-ethyl-7-(methylamino)-2-oxo-1,2-dihydro-1,6-naphthyridin-3-yl]-2- fluorophenyl]-3-phenylurea or a pharmaceutically acceptable salt thereof, or 1-(5-(7-amino-1-ethyl-2-oxo-1,2-dihydro-1,6-naphthyridin-3-yl)-4-bromo-2-fluorophenyl)-3-phenylurea or a pharmaceutically acceptable salt thereof; and (ii) a MAPKAP kinase inhibitor, e.g., the MEK inhibitor trametinib, binimetinib, or cobimetinib; the ERK inhibitor ulixertinib, or a RAF inhibitor. In particular embodiments of any of the methods disclosed herein, the tumor is a c-KIT-mediated solid tumor, e.g., a c-KIT-mediated GIST or melanoma.

### Definitions

Compounds A and B as used herein refer to 1-[4-bromo-5-[1-ethyl-7-(methylamino)-2-oxo-1,2-dihydro-1,6-naphthyridin-3-yl]-2-fluorophenyl]-3-phenylurea and 1-(5-(7-amino-1-ethyl-2-oxo-1,2-dihydro-1,6-naphthyridin-3-yl)-4-bromo-2-fluorophenyl)-3-phenylurea, respectively. Pharmaceutically acceptable salts, tautomers, hydrates, and solvates, of Compounds A and B are also contemplated in this disclosure. The structures of Compounds A and B are represented below:

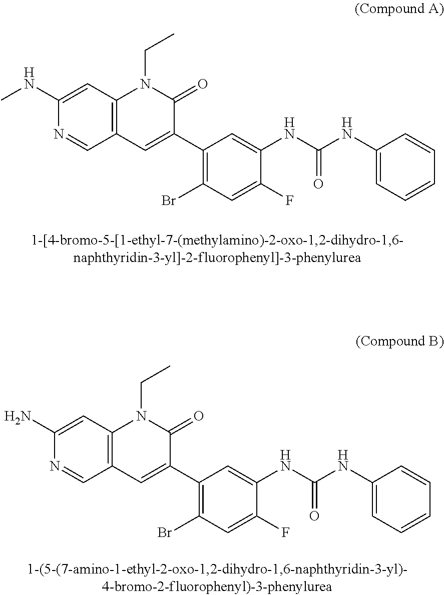 [SMILES: CCn1c(=O)c(-c2cc(NC(=O)Nc3ccccc3)c(F)cc2Br)cc2cnc(NC)cc21 ; CCn1c(=O)c(-c2cc(NC(=O)Nc3ccccc3)c(F)cc2Br)cc2cnc(N)cc21]

Methods of making Compound A and Compound B are disclosed in U.S. Pat. No. 8,461,179B1 the contents of which are incorporated herein by reference.

Illustrative methods and materials are now described. In the specification and the appended claims, the singular forms also include the plural unless the context clearly dictates otherwise. Unless defined otherwise, all technical and scientific terms used herein have the same meaning as commonly understood by one of ordinary skill in the art.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes acid addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

A “pharmaceutical composition” refers to a formulation of a compound described herein, e.g., Compound A or a pharmaceutically acceptable salt thereof, and a medium generally accepted in the art for the delivery of the biologically active compound to mammals, e.g., humans. Such a medium includes all pharmaceutically acceptable carriers, diluents or excipients therefor.

Subjects or patients “in need of treatment” with a combination therapy of the present disclosure, e.g., Compound A in combination with a MEK inhibitor, include patients with diseases and/or conditions that can be treated with a combination disclosed herein to achieve a beneficial therapeutic result, e.g., a GIST patient. A beneficial outcome includes an objective response, increased progression free survival, increased survival, prolongation of stable disease, and/or a decrease in the severity of symptoms or delay in the onset of symptoms. In certain embodiments, a patient in need of treatment is suffering from a tumor growth or tumor progression; the patient is suffering from, but not limited to, lung adenocarcinoma, squamous cell lung cancer, glioblastoma, pediatric glioma, astrocytomas, sarcomas, melanoma or gastrointestinal stromal tumors.

The term “effective amount” when used in connection with a compound or other therapeutic agent disclosed herein, refers to an amount of the therapeutic agent, e.g., Compound A or a MEK inhibitor, alone or in combination, that is useful to treat or prevent a disease or disorder. The effective amount of therapeutic agents used in a combination therapy is the amount of each of the therapeutic agents that is useful for treating or preventing a disease or disorder when used in the combination therapy, even if the amount of one or both of the therapeutic agents, in the absence of the other therapeutic agent, is ineffective to treat or prevent the disease or disorder. In certain embodiments, an effective amount is a quantity that results in prolonged cell stasis of GIST cells, cytocidal GIST cell killing, apoptosis of GIST cells, eradication of GIST cells, regression of a GIST, reduction of GIST tumor volume, inhibition of GIST regrowth, and/or leads to enhanced cell stasis, cell death, apoptosis, or eradication to the limit of detection, of GIST cancer cell lines that are resistant to imatinib in combination with a MEK inhibitor, and/or a leads to a beneficial clinical outcome of the condition being treated with the compound compared with the absence of treatment. The “effective amount” can vary depending upon the mode of administration, specific locus of the disease or disorder, and the age, body weight, and general health of the subject. The amount of the compounds administered will depend on the degree, severity, and type of the disease or condition, the amount of therapy desired, and the release characteristics of the pharmaceutical formulation(s). It will also depend on the subject's health, size, weight, age, sex and tolerance to drugs. Typically, the compounds are administered for a sufficient period of time to achieve the desired therapeutic effect.

The terms “treatment,” “treat,” and “treating,” are meant to include the full spectrum of intervention in patients with “cancer” with the intention to induce prolonged cell stasis of GIST cells, to induce cytocidal GIST cell killing, to induce apoptosis of GIST cells, to induce eradication of GIST tumor cells to the limit of visual detection as determined by 5X objective microscopy, to cause regression of a GIST tumor in a patient, to reduce GIST tumor volume, to inhibit GIST regrowth, and/or to inhibit the growth of resistant GIST cells on a given treatment, such as administration of a combination therapy disclosed herein to alleviate, slow or reverse one or more of the symptoms and to induce regression of the GIST even if the GIST is not actually eliminated. In some embodiments, treatment includes eliminating the disease or disorder, e.g., GISTs, entirely. Treating can be curing, improving, or at least partially ameliorating the disorder.

“Cancer” as defined herein refers to a new growth which has the ability to invade surrounding tissues, metastasize (spread to other organs) and which may eventually lead to the patient's death if untreated. In certain embodiments, a cancer” can be a solid tumor.

“Tumor” as used herein refers to a mass. This is a term that may refer to benign (generally harmless) or malignant (cancerous) growths. Malignant growth can originate from a solid organ or the bone marrow.

“Tumor growth” as defined herein refers to growth of a mass caused by genomic alterations of a c-KIT gene, which may alter c-KIT protein expression and/or activity.

“Tumor progression” as defined herein refers to growth of an existing c-KIT dependent tumor, e.g., a GIST, wherein such growth of an existing mass may be caused by further genomic alterations of c-KIT resistant to a treatment.

“Tumor regression”, “complete response” and “partial response” as defined herein refer to a reduction in tumor size as determined by weight or volume as determined by RECIST 1.1 or Choi criteria.

Eradication of an existing c-KIT-mediated tumor, e.g., a c-KIT-mediated GIST, is defined as a “complete cytocidal cell killing” of a tumor to the limit of detection as determined by 5X objective microscopy for in vitro evaluations, or as defined as a complete response as determined by RECIST 1.1 or Choi criteria for in vivo preclinical or clinical evaluations without the possibility of regrowth of the tumor under preclinical or clinical conditions. Accordingly, “eradication of a c-KIT-mediated tumor” indicates that all cells of the c-KIT-mediated tumor are killed or removed to the limit of detection without the possibility of regrowth of the c-KIT-mediated tumor.

“Tumor regrowth” as used herein refers to growth of a tumor that previously halted growth or regressed following a treatment, e.g., treatment with Gleevec® or Sutent®. In certain embodiments, tumor regrowth occurs due to the introduction of a c-Kit secondary mutation in a tumor cell. In other embodiments, tumor regrowth occurs due to the activation or mutation of a different signaling pathway, including but not limited to activation of the MAPKAP signaling pathway, which includes signaling through MEK kinases.

“Cell stasis” as used herein refers to cells ceasing to divide and remaining in a dormant non-replicative state.

“Apoptosis” as used herein refers to programmed cell death. Features of apoptosis detectable by histologic and histochemical methods include cell shrinkage; increased membrane permeability; nuclear and cytoplasmic condensation; endolytic cleavage of nuclear DNA into oligonucleosomal fragments; and ultimately formation of apoptotic bodies, which are absorbed and removed by macrophages. Apoptosis is primarily medated by the caspases, which are aspartate-specific serine proteases. Apoptosis can be induced via intrinsic genetic programming in response to various conditions, e.g., DNA damage or growth factor withdrawal, or apoptosis can be induced by extrinsic factors, such as injury to cellular DNA by irradiation and some cytotoxic agents used to treat cancer. It can be suppressed by naturally occurring factors (for example, cytokines) and by some drugs (for example, protease inhibitors). Apoptosis typically does not occur or is compromised in malignant cells. In particular embodiments, apoptosis refers to programmed cell death as determined by increases in cleaved and activated caspase 3 and caspase 7.

A “combination therapy” is a treatment that includes the administration of two or more therapeutic agents, e.g., Compound A and a MEK inhibitor, to a patient. The two or more therapeutic agents may be delivered at the same time, e.g., in separate pharmaceutical compositions or in the same pharmaceutical composition, or they may be delivered at different times. For example, they may be delivered concurrently or during overlapping time periods, and/or one therapeutic agent may be delivered before or after the other therapeutic agent(s). Treatment with a combination of a KIT inhibitor such as Compound A and a MEK inhibitor optionally includes treatment with either single agent, preceded or followed by a period of concurrent treatment with both agents. However, it is contemplated that during some time period, effective amounts of the two or more therapeutic agents are present within the patient.

A “MAPKAP pathway inhibitor” is an inhibitor of the MAP kinase signaling pathway. Inhibitors of this pathway include RAS inhibitors, RAF inhibitors (e.g. dabrafenib, vemurafenib, LY3009120), MEK inhibitors (e.g. trametinib, binimetinib, cobimetinib), and ERK inhibitors (e.g. ulixertinib). The terms “MAPKAP pathway inhibitor” and “MAPKAP kinase inhibitor are used interchangeably herein.

### Methods of Treatment

The compounds and compositions described herein can be used to treat tumors in a patient in need thereof. For example, provided herein is a method of treating a tumor having one or more c-KIT mutations in patient in need thereof, comprising administering to the patient: an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and an effective amount of one or more MAPKAP kinase inhibitors. In one embodiment, the MAPKAP kinase inhibitor is selected from the group consisting of a mitogen-activated protein kinase inhibitor (MEK inhibitor) and an effective amount of an extracellular signal regulated kinase inhibitor (ERK inhibitor).

The c-KIT mutation can be a primary mutation in exon 9, exon 11, exon 13, or exon 17 of the c-KIT gene. In another embodiment, the c-KIT mutation is a deletion mutation.

Furthermore, the tumor can have one or more secondary resistance mutations in the c-KIT gene. In some embodiments, the secondary resistance mutation is in exon 13, exon 14, exon 17, or exon 18 of the c-KIT gene. In some embodiments, the secondary resistance mutation is in exon 17 of the c-KIT gene. In some embodiments, the secondary resistance mutation is the substitution of aspartic acid in codon 816 or the substitution of asparagine in codon 822. In some embodiments, the secondary resistance mutation is one of D816V, D816E, D816H, D820A, T670I, or N822V. In some embodiments, the secondary resistance mutation was acquired after previous administration of imatinib, sunitib or regorafenib, or a pharmaceutically acceptable salt thereof to the patient.

Such a disclosed method further comprises determining if the tumor has the c-KIT secondary mutation. In some embodiments, determining if the tumor has the c-KIT secondary mutation comprises identifying mutations in DNA extracted from a tumor sample. In some embodiments, determining if the tumor has the c-KIT secondary mutation comprises identifying mutations in circulating tumor DNA. In another embodiment, the tumor was been resistant to treatment with imatinib mesylate, sunitinib malate, or regorafenib.

Furthermore, the tumor can be selected from the group consisting of lung adenocarcinoma, squamous cell lung cancer, glioblastoma, pediatric glioma, astrocytoma, sarcoma, gastrointestinal stromal tumor (GIST), and melanoma. In some embodiments, the tumor is melanoma. In some embodiments, the tumor is GIST.

The method may further comprise administering to the patient a cancer targeted therapeutic agent, cancer-targeted biological, immune checkpoint inhibitor, and/or chemotherapeutic agent. The method may also further comprise administering a RAF inhibitor to the patient.

In another embodiment, the 1-[4-bromo-5-[1-ethyl-7-(methylamino)-2-oxo-1,2-dihydro-1,6-naphthyridin-3-yl]-2-fluorophenyl]-3-phenylurea, or the pharmaceutically acceptable salt thereof, and the MAPKAP kinase inhibitor is administered substantially concurrently or sequentially.

The MEK inhibitor in this disclosed method can be selected from the group consisting of trametinib, selumetinib, cobimetinib, and binimetinib. In some embodiments, the MEK inhibitor is binimetinib. In some embodiments, the MEK inhibitor is trametinib. In some embodiments, the ERK inhibitor is selected from the group consisting of ulixertinib, SCH772984, and LY3214996.

Administration for two weeks or more in accordance with such a disclosed method can result in the patient having partial reduction in tumor volume of at least 30%. In some embodiments, the treatment results in a complete reduction in tumor volume.

The disclosed method may further comprise determining if the tumor or tumor cells comprise a primary c-KIT gene mutation. In some embodiments, the primary mutation is in exon 11 of the c-KIT gene. In some embodiments, the primary mutation is in exon 9 of the c-KIT gene. In some embodiments, the primary mutation is a deletion mutation. In some embodiments, the primary mutation is V560D. In other embodiments, one or more additional secondary mutations c-KIT mutations are present.

Also provided by the disclosure is a method of treating a solid tumor in an imatinib resistant patient, comprising: administering to the patient an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and administering to the patient an effective amount of a MAPKAP kinase inhibitor selected from the group consisting of trametinib, binimetinib, cobimetinib, and ulixertinib, wherein the solid tumor is selected from the group consisting of of lung adenocarcinoma, squamous cell lung cancer, glioblastoma, pediatric glioma, astrocytoma, sarcoma, gastrointestinal stromal tumor (GIST), and melanoma. In some embodiments, the method further comprises administering a RAF inhibitor. In some embodiments, the RAF inhibitor is a pan-RAF inhibitor.

Also provided herein is a method of treating an imatinib resistant gastrointestinal stromal tumor or imatinib resistant melanoma in a patient in need thereof, comprising administering to the patient an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and administering to the patient an effective amount of a MAPKAP kinase inhibitor selected from the group consisting of trametinib, binimetinib, cobimetinib, and ulixertinib.

In some embodiments, the method further comprises determining whether the tumor has a mutation of the c-KIT gene. In some embodiments, the mutation is in exon 17 of the c-KIT gene. In some embodiments, the c-KIT mutation is the substitution of aspartic acid in codon 816 or the substitution of asparagine in codon 822. In some embodiments, the mutation is one of D816V, D816E, D816H, D820A, T670I, or N822V.

Additionally provided is a method of treating a solid tumor in a patient need thereof, comprising administering to the patient an effective amount of 1-[4-bromo-5-[1-ethyl-7-(methylamino)-2-oxo-1,2-dihydro-1,6-naphthyridin-3-yl]-2- fluorophenyl]-3-phenylurea, or a pharmaceutically acceptable salt thereof; and administering to the patient an effective amount of a RAF inhibitor.

In such a disclosed method, the solid tumor can be selected from the group consisting of lung adenocarcinoma, squamous cell lung cancer, GIST, and melanoma. In some embodiments, the solid tumor has one or more mutations of the c-KIT gene.

Furthermore, the RAF inhibitor can be a pan-RAF inhibitor. In another embodiment, the RAF inhibitor is dabrafenib, vemurafenib, or LY3009120.

Also provided by the disclosure is a method of treating a solid tumor in a patient in need thereof, comprising: administering to the patient an effective amount of 1-[4-bromo-5-[1-ethyl-7-(methylamino)-2-oxo-1,2-dihydro-1,6-naphthyridin-3-yl]- 2-fluorophenyl]-3-phenylurea, or a pharmaceutically acceptable salt thereof; and administering to the patient an effective amount of a MAPKAP kinase inhibitor selected from the group consisting of trametinib, binimetinib, cobimetinib, and ulixertinib, wherein the solid tumor is selected from the group consisting of of lung adenocarcinoma, squamous cell lung cancer, glioblastoma, pediatric glioma, astrocytoma, sarcoma, gastrointestinal stromal tumor (GIST), and melanoma. In some embodiments, the method further comprises administering a RAF inhibitor. In some embodiments, the RAF inhibitor is a pan-RAF inhibitor.

Also provided herein is a method of treating a gastrointestinal stromal tumor or melanoma in a patient in need thereof, comprising administering to the patient an effective amount of 1-[4-bromo-5-[1-ethyl-7-(methylamino)-2-oxo-1,2-dihydro-1,6-naphthyridin-3-y1]-2-fluorophenyl]-3-phenylurea, or a pharmaceutically acceptable salt thereof; and administering to the patient an effective amount of a MAPKAP kinase inhibitor selected from the group consisting of trametinib, binimetinib, cobimetinib, and ulixertinib.

In some embodiments, the method further comprises determining whether the tumor has a mutation of the c-KIT gene. In some embodiments, the mutation is in exon 17 of the c-KIT gene. In some embodiments, the c-KIT mutation is the substitution of aspartic acid in codon 816 or the substitution of asparagine in codon 822. In some embodiments, the mutation is one of D816V, D816E, D816H, D820A, T670I, or N822V.

In one embodiment, the present disclosure provides methods of treating or preventing a tumor in a patient, optionally a c-KIT-mediated tumor, e.g., a GIST, comprising administering to a patient in need thereof an effective amount of Compound A, or a pharmaceutically acceptable salt thereof, in combination with an effective amount of a MEK inhibitor, e.g., trametinib. In a related embodiment, the present disclosure provides methods of treating or preventing a tumor in a patient, optionally a c-KIT-mediated tumor, e.g., a GIST, comprising administering to a patient in need thereof an effective amount of Compound B, or a pharmaceutically acceptable salt thereof, in combination with an effective amount of a MEK inhibitor, e.g., trametinib.

In specific embodiments, these methods include methods for: inducing prolonged stasis of tumor cells, e.g., GIST cells; killing of tumor cells, e.g., GIST cells; inducing apoptosis of tumor cells, e.g., GIST cells; inducing tumor cell eradication to the limit of detection, e.g., GIST cells; inducing tumor regression, e.g., GIST regression; reducing tumor volume, e.g., GIST tumor volume; inhibiting tumor regrowth, e.g., GIST regrowth. In another specific embodiment, these methods include methods for inducing prolonged stasis of tumor cells, e.g., GIST cells. In another specific embodiment, these methods include methods killing of tumor cells, e.g., GIST cells. In another specific embodiment, these methods include methods inducing apoptosis of tumor cells, e.g., GIST cells. In another specific embodiment, these methods include methods for inducing tumor cell eradication to the limit of detection, e.g., GIST cells. In another specific embodiment, these methods include methods for inducing tumor regression, e.g., GIST regression. In another specific embodiment, these methods include methods for reducing tumor volume, e.g., GIST tumor volume. In another specific embodiment, these methods include methods for inhibiting tumor regrowth, e.g., GIST regrowth. In another specific embodiment, these methods include methods for inhibiting the growth of drug-resistant tumor cells, e.g., drug-resistant GIST cells. In certain embodiments, the methods encompass methods for eradicating a tumor to the limit of detection, e.g., a GIST, in a subject. In particular embodiments of any of the methods disclosed herein, tumor growth or tumor progression in the patient is caused by c-KIT overexpression, constitutive phosphorylation of c-KIT, increased c-KIT activity, oncogenic c-KIT missense mutations, oncogenic deletion c-KIT mutations, oncogenic nucleotide duplications/insertions, oncogenic c-KIT gene rearrangements leading to c-KIT fusion proteins, c-KIT intragenic in-frame deletions, and/or oncogenic c-KIT gene amplification. In one embodiment, the tumor growth or tumor progression is caused by constitutive phosphorylation of c-KIT. In particular embodiments, the tumor comprises one or more of the primary activating c-KIT mutations and/or secondary c-KIT mutations disclosed herein. In another particular embodiment, the tumor comprises one or more mutations in genes other then c-KIT that cause tumor growth by signaling through the MAPKAP pathway involving RAF, MEK, or ERK kinase activation.

Where the methods described herein refer to treatment with Compound A or a pharmaceutically acceptable salt thereof, or Compound B or a pharmaceutically acceptable salt thereof, it is meant that only one of Compound A or a pharmaceutically acceptable salt thereof, or Compound B or a pharmaceutically acceptable salt thereof is required. However, it is understood that these methods encompass administering to a patient both Compound A or a pharmaceutically acceptable sale thereof, and Compound B or a pharmaceutically acceptable salt thereof, in combination with a MEK inhibitor, ERK inhibitor, or RAF inhibitor. Furthermore, it is understood that upon administration of Compound A in combination with a MEK inhibitor, ERK inhibitor, or a RAF inhibitor to a subject, some amount of the Compound A is metabolized in vivo to Compound B, and that an in vivo mixture of Compound A and Compound B may also be used to effectively treat a subject in combination with the MEK inhibitor, the ERK inhibitor or the RAF inhibitor.

Illustrative MEK inhibitors that may be used according to the disclosed methods and compositions include, but are not limited to, trametinib, selumetinib, cobimetinib, and binimetinib.

Illustrative ERK inhibitors that may be used according to the disclosed methods and compositions include, but are not limited to, ulixertinib, SCH772984,LY3214996, ravoxertinib, and VX-11e.

Illustrative RAF inhibitors that may be used according to the disclosed methods and compositions include, but are not limited to, LY3009120, dabrafenib, and vemurafenib.

In one embodiment, Compound A or a pharmaceutically acceptable salt thereof and a MEK inhibitor, e.g., trametinib, are administered to a patient with a c-KIT-mediated tumor, e.g., a GIST. In another embodiment, Compound B or a pharmaceutically acceptable salt thereof and a MEK inhibitor, e.g., trametinib, are administered to a patient with a c-KIT-mediated tumor, e.g., a GIST.

In a related embodiment, Compound A or a pharmaceutically acceptable salt thereof and a MEK inhibitor, e.g., trametinib, are administered to a patient with a tumor, e.g., a patient having a GIST, wherein tumor growth or tumor progression is caused by a primary activating c-KIT mutation and/or a secondary c-KIT mutation. In another embodiment, Compound B or a pharmaceutically acceptable salt thereof and a MEK inhibitor, e.g., trametinib, are administered to a patient with a tumor, e.g., a patient having a GIST, wherein tumor growth or tumor progression is caused by a primary activating c-KIT mutation and/or a secondary c-KIT mutation. In certain embodiments, the primary activating c-KIT mutation is an exon 11 mutation (e.g., a 57 base pair exon 11 deletion). In certain embodiments, the primary activating c-KIT mutation is an exon 9 A-Y 502-503 duplication. In certain embodiments, the primary activating c-KIT mutation is an exon 13 mutation. In certain embodiments, the primary activating c-KIT mutation is an exon 17 mutation. In certain embodiments, the secondary c-KIT mutation is any disclosed resistance mutation herein, e.g., a T670I mutation or a D816E mutation. In certain embodiments, multiple secondary c-KIT resistance mutations coexist in a subject.

In certain embodiments, Compound A or a pharmaceutically acceptable salt thereof and a MEK inhibitor, e.g., trametinib, are administered to a cancer patient. In certain embodiments, Compound B or a pharmaceutically acceptable salt thereof and a MEK inhibitor, e.g., trametinib, are administered to a cancer patient. In particular embodiments of any of the methods disclosed herein, the tumor or cancer is lung adenocarcinoma, squamous cell lung cancer, glioblastoma, pediatric glioma, astrocytomas, sarcomas, melanoma, or gastrointestinal stromal tumors (GIST). In one embodiment, the cancer is melanoma. In another embodiment, the tumor or cancer is a gastrointestinal stromal tumor (GIST). In particular embodiments of any of these methods, the tumor or cancer is a c-KIT-mediated cancer, e.g., a c-KIT-mediated GIST or melanoma.

Treatment with Compound A or a pharmaceutically acceptable salt thereof, or Compound B or a pharmaceutically acceptable salt thereof, in combination with a MEK inhibitor, e.g., trametinib, encompasses administering Compound A or a pharmaceutically acceptable salt thereof, or Compound B or a pharmaceutically acceptable salt thereof, before, after, simultaneous with, or during an overlapping time period with administering the MEK inhibitor. It is understood that an effective amount of any of Compound A or a pharmaceutically acceptable salt thereof, Compound B or a pharmaceutically acceptable salt thereof, or a MEK inhibitor, e.g., trametinib, may be different when used in the combinations disclosed herein as compared to when any of these agents is used by itself for the same purpose, e.g., to treat or prevent a tumor. In particular embodiments, an effective amount of Compound A or a pharmaceutically acceptable salt thereof, or of Compound B or a pharmaceutically acceptable salt thereof, is a lower amount when administered as a combination therapy with a MEK inhibitor, e.g., trametinib, as compared to when it is administered as a monotherapy, e.g., to treat or prevent a GIST. In particular embodiments, an effective amount of a MEK inhibitor, e.g., trametinib, is a lower amount when administered in a combination therapy with Compound A or a pharmaceutically acceptable salt thereof, or when administered in a combination therapy with Compound B or a pharmaceutically acceptable salt thereof, e.g., to treat or prevent a GIST.

Any of the methods disclosed herein may further include determining that the tumor being treated has one or more c-KIT gene mutations. Such a determination may be made by routine methods for determining the presence of a gene mutation in a biological sample, e.g., a tumor sample, a blood sample, or a plasma sample obtained from the patient. In addition, such a determination may be made by reviewing the results of tests performed to determine the presence of one or more c-KIT gene mutations in a biological sample, e.g., a tumor sample, blood sample, or plasma sample obtained from the patient. In certain embodiments of any of the methods disclosed herein, the methods are performed on patients wherein the tumor has been identified as having one or more c-KIT gene mutations. The c-KIT gene mutations include but are not limited to any of those specifically described herein.

In various aspects of any of the methods disclosed herein, treatment with either Compound A or a pharmaceutically acceptable salt thereof, or Compound B or a pharmaceutically acceptable salt thereof, in combination with a MEK inhibitor, e.g., trametinib: induces prolonged cell stasis of tumor cells, e.g. GIST cells; induces killing of tumor cells, e.g., GIST cells; induces apoptosis of tumor cells, e.g., GIST cells; induces tumor cell eradication to the limit of detection, e.g., GIST cells; induces tumor regression, e.g., GIST tumor; reduces tumor weight or volume; e.g., GIST tumor; inhibits tumor regrowth, e.g., GIST tumor. In another aspect of any of the methods disclosed herein, treatment with either Compound A or a pharmaceutically acceptable salt thereof, or Compound B or a pharmaceutically acceptable salt thereof, in combination with a MEK inhibitor, e.g., trametinib: induces prolonged cell stasis of tumor cells, e.g. GIST cells; induces killing of tumor cells, e.g., GIST cells; induces apoptosis of tumor cells, e.g., GIST cells; induces tumor cell eradication to the limit of detection, e.g., GIST cells; induces tumor regression, e.g., GIST tumor; reduces tumor weight or volume; e.g., GIST tumor; inhibits tumor regrowth, e.g., GIST tumor in a drug resistant tumor, e.g. drug-resistant GIST. In another aspect of any of the methods disclosed herein, treatment with either Compound A or a pharmaceutically acceptable salt thereof, or Compound B or a pharmaceutically acceptable salt thereof, in combination with a MEK inhibitor, e.g., trametinib: eradicates a tumor to the limit of detection, in a patient being treated, e.g., a GIST patient. Methods for measuring or determining amounts of tumor cell stasis, tumor cell death, apoptosis of tumor cells, tumor regression, tumor weight or volume, tumor regrowth, growth of resistant tumor cells, and eradication of tumors are known in the art and include any methods described herein.

In particular embodiments, treatment with a combination of: Compound A or a pharmaceutically acceptable salt thereof, or Compound B or a pharmaceutically acceptable salt thereof; and a MEK inhibitor, e.g., trametinib, results in an increased amount of tumor cell stasis, killing of tumor cells or apoptosis of tumor cells, e.g., GIST cells, as compared to the amount of stasis, cell killing or apoptosis of tumor cells of the same type or same tumor type either untreated or treated with only a MEK inhibitor, e.g., trametinib, or with only a c-KIT inhibitor, e.g., imatinib, or with a combination of a MEK inhibitor, e.g., trametinib, with the c-KIT inhibitor imatinib. For example, cell stasis, cell killing or apoptosis may be increased by at least 10%, at least 20%, at least 30%, at least 40%, at least 50%, at least 60%, at least 70%, at least 80%, at least 90%, at least two-fold, at least three-fold, at least four-fold, at least five-fold, at least 10-fold, or at least 20-fold. In certain embodiments, amounts of apoptosis are determined by measuring caspase activity of tumor cells.

In particular embodiments, treatment with a combination of: Compound A or a pharmaceutically acceptable salt thereof, or Compound B or a pharmaceutically acceptable salt thereof; and a MEK inhibitor, e.g., trametinib, results in increased tumor regression or decreased tumor size or volume (e.g., a GIST), as compared to the size, e.g., weight or volume of a tumor of the same type or the same tumor either untreated or treated with only a MEK inhibitor, e.g., trametinib, or with only a c-KIT inhibitor, e.g., imatinib. For example, tumor weight or volume may be decreased by at least 10%, at least 20%, at least 30%, at least 40%, at least 50%, at least 60%, at least 70%, at least 80%, or at least 90%.

In particular embodiments, treatment with a combination of: Compound A or a pharmaceutically acceptable salt thereof, or Compound B or a pharmaceutically acceptable salt thereof; and a MEK inhibitor, e.g., trametinib, inhibits the amount of tumor growth or regrowth, e.g., GIST growth or regrowth, to a greater extent as compared to the amount of growth or regrowth the same type or the same tumor either untreated or treated with only a MEK inhibitor, e.g., trametinib, or with only a c-KIT inhibitor, e.g., imatinib. For example, tumor growth or regrowth may be inhibited by at least 10%, at least 20%, at least 30%, at least 40%, at least 50%, at least 60%, at least 70%, at least 80%, or at least 90%.

In particular embodiments, treatment with a combination of: Compound A or a pharmaceutically acceptable salt thereof, or Compound B or a pharmaceutically acceptable salt thereof; and a MEK inhibitor, e.g., trametinib, inhibits the growth of resistant tumor cells, e.g., resistant GIST cells, to a greater extent as compared to the amount of growth of resistant tumor cells of the same type or the same tumor either untreated or treated with only a MEK inhibitor, e.g., trametinib, or with only a c-KIT inhibitor, e.g., imatinib, or with a combination of a MEK inhibitor, e.g., trametinib and a c-KIT inhibitor, e.g., imatinib. For example, the amount of growth or number of resistant tumor cells may be inhibited by at least 10%, at least 20%, at least 30%, at least 40%, at least 50%, at least 60%, at least 70%, at least 80%, or at least 90%. In particular embodiments, resistant tumor cells are resistant to treatment with a c-KIT inhibitor, e.g., imatinib, and/or a MEK inhibitor, e.g., trametinib. In certain embodiments, the resistant tumor cells comprise a c-KIT secondary mutation. In certain embodiments, the c-KIT secondary mutation is a mutation of any of the following amino acid residues of c-KIT: V654, N655, T670, L783, D816, D820, N822, Y823, A829, and/or T847,including but not limited to any of the amino acid substitutions depicted in the accompanying figures. In particular embodiments, the resistant tumor cells comprise an activated MAPKAP kinase pathway, and in certain embodiments, they comprise a mutation in a mutation in a RAS gene, e.g., an N-RAS or K-RAS gene, a Fibloblast Growth Factor Receptor (FGFR) gene, and/or a Neurofibromin-1 (NF1) gene. In certain embodiments, the mutation is an N-RAS G12D mutation.

In particular embodiments, treatment with a combination of: either Compound A or a pharmaceutically acceptable salt thereof, or Compound B or a pharmaceutically acceptable salt thereof; in combination with a MEK inhibitor, e.g., trametinib, results in eradication of a tumor to the limit of detection, e.g., a GIST. In particular embodiments, eradication of a tumor means there is no longer any detectable tumor in the patient to the limit of detection. In particular embodiments, there is no detectable tumor in the patient for at least six months, at least one year, at least two years, at least five years, or at least 10 years following eradication of the tumor, e.g., GIST, by a combination therapy disclosed herein. Tumor eradication may be determined by photon emission tomography (PET), CT scans, absence of circulating cell free DNA (cfDNA) containing a c-KIT mutation, absence of circulating tumor cells (CTCs) present in the vasculature of a subject, or absence of a cancer cell biomarker within the circulating blood vasculature of a subject.

In various aspects of any of the methods disclosed herein, treatment with either Compound A or a pharmaceutically acceptable salt thereof, or Compound B or a pharmaceutically acceptable salt thereof, in combination with a ERK inhibitor, e.g., ulixertinib: induces prolonged cell stasis of tumor cells, e.g. GIST cells; induces killing of tumor cells, e.g., GIST cells; induces apoptosis of tumor cells, e.g., GIST cells; induces tumor cell eradication to the limit of detection, e.g., GIST cells; induces tumor regression, e.g., GIST tumor; reduces tumor weight or volume; e.g., GIST tumor; inhibits tumor regrowth, e.g., GIST tumor. In another aspect of any of the methods disclosed herein, treatment with either Compound A or a pharmaceutically acceptable salt thereof, or Compound B or a pharmaceutically acceptable salt thereof, in combination with a ERK inhibitor, e.g., ulixertinib: induces prolonged cell stasis of tumor cells, e.g. GIST cells; induces killing of tumor cells, e.g., GIST cells; induces apoptosis of tumor cells, e.g., GIST cells; induces tumor cell eradication to the limit of detection, e.g., GIST cells; induces tumor regression, e.g., GIST tumor; reduces tumor weight or volume; e.g., GIST tumor; inhibits tumor regrowth, e.g., GIST tumor in a drug resistant tumor, e.g. drug-resistant GIST. In another aspect of any of the methods disclosed herein, treatment with either Compound A or a pharmaceutically acceptable salt thereof, or Compound B or a pharmaceutically acceptable salt thereof, in combination with a ERK inhibitor, e.g., ulixertinib: eradicates a tumor to the limit of detection, in a patient being treated, e.g., a GIST patient. Methods for measuring or determining amounts of tumor cell stasis, tumor cell death, apoptosis of tumor cells, tumor regression, tumor weight or volume, tumor regrowth, growth of resistant tumor cells, and eradication of tumors are known in the art and include any methods described herein.

In particular embodiments, treatment with a combination of: Compound A or a pharmaceutically acceptable salt thereof, or Compound B or a pharmaceutically acceptable salt thereof; and a ERK inhibitor, e.g., ulixertinib, results in an increased amount of tumor cell stasis, killing of tumor cells or apoptosis of tumor cells, e.g., GIST cells, as compared to the amount of stasis, cell killing or apoptosis of tumor cells of the same type or same tumor type either untreated or treated with only a ERK inhibitor, e.g., ulixertinib, or with only a c-KIT inhibitor, e.g., imatinib, or with a combination of a ERK inhibitor, e.g., ulixertinib, with the c-KIT inhibitor imatinib. For example, cell stasis, cell killing or apoptosis may be increased by at least 10%, at least 20%, at least 30%, at least 40%, at least 50%, at least 60%, at least 70%, at least 80%, at least 90%, at least two-fold, at least three-fold, at least four-fold, at least five-fold, at least 10-fold, or at least 20-fold. In certain embodiments, amounts of apoptosis are determined by measuring caspase activity of tumor cells.

In particular embodiments, treatment with a combination of: Compound A or a pharmaceutically acceptable salt thereof, or Compound B or a pharmaceutically acceptable salt thereof; and a ERK inhibitor, e.g., ulixertinib, results in increased tumor regression or decreased tumor size or volume (e.g., a GIST), as compared to the size, e.g., weight or volume of a tumor of the same type or the same tumor either untreated or treated with only a ERK inhibitor, e.g., ulixertinib, or with only a c-KIT inhibitor, e.g., imatinib. For example, tumor weight or volume may be decreased by at least 10%, at least 20%, at least 30%, at least 40%, at least 50%, at least 60%, at least 70%, at least 80%, or at least 90%.

In particular embodiments, treatment with a combination of: Compound A or a pharmaceutically acceptable salt thereof, or Compound B or a pharmaceutically acceptable salt thereof; and a ERK inhibitor, e.g., ulixertinib, inhibits the amount of tumor growth or regrowth, e.g., GIST growth or regrowth, to a greater extent as compared to the amount of growth or regrowth the same type or the same tumor either untreated or treated with only a ERK inhibitor, e.g., ulixertinib, or with only a c-KIT inhibitor, e.g., imatinib. For example, tumor growth or regrowth may be inhibited by at least 10%, at least 20%, at least 30%, at least 40%, at least 50%, at least 60%, at least 70%, at least 80%, or at least 90%.

In particular embodiments, treatment with a combination of: Compound A or a pharmaceutically acceptable salt thereof, or Compound B or a pharmaceutically acceptable salt thereof; and a ERK inhibitor, e.g., ulixertinib, inhibits the growth of resistant tumor cells, e.g., resistant GIST cells, to a greater extent as compared to the amount of growth of resistant tumor cells of the same type or the same tumor either untreated or treated with only a ERK inhibitor, e.g., ulixertinib, or with only a c-KIT inhibitor, e.g., imatinib, or with a combination of a ERK inhibitor, e.g., ulixertinib and a c-KIT inhibitor, e.g., imatinib. For example, the amount of growth or number of resistant tumor cells may be inhibited by at least 10%, at least 20%, at least 30%, at least 40%, at least 50%, at least 60%, at least 70%, at least 80%, or at least 90%. In particular embodiments, resistant tumor cells are resistant to treatment with a c-KIT inhibitor, e.g., imatinib, and/or a ERK inhibitor, e.g., ulixertinib. In certain embodiments, the resistant tumor cells comprise a c-KIT secondary mutation. In certain embodiments, the c-KIT secondary mutation is a mutation of any of the following amino acid residues of c-KIT: V654, N655, T670, L783, D816, D820, N822, Y823, A829, and/or T847,including but not limited to any of the amino acid substitutions depicted in the accompanying figures. In particular embodiments, the resistant tumor cells comprise an activated MAPKAP kinase pathway, and in certain embodiments, they comprise a mutation in a mutation in a RAS gene, e.g., an N-RAS or K-RAS gene, a Fibloblast Growth Factor Receptor (FGFR) gene, and/or a Neurofibromin-1 (NF1) gene. In certain embodiments, the mutation is an N-RAS G12D mutation.

In particular embodiments, treatment with a combination of: either Compound A or a pharmaceutically acceptable salt thereof, or Compound B or a pharmaceutically acceptable salt thereof; in combination with a ERK inhibitor, e.g., ulixertinib, results in eradication of a tumor to the limit of detection, e.g., a GIST. In particular embodiments, eradication of a tumor means there is no longer any detectable tumor in the patient to the limit of detection. In particular embodiments, there is no detectable tumor in the patient for at least six months, at least one year, at least two years, at least five years, or at least 10 years following eradication of the tumor, e.g., GIST, by a combination therapy disclosed herein. Tumor eradication may be determined by photon emission tomography (PET), CT scans, absence of circulating cell free DNA (cfDNA) containing a c-KIT mutation, absence of circulating tumor cells (CTCs) present in the vasculature of a subject, or absence of a cancer cell biomarker within the circulating blood vasculature of a subject.

In various aspects of any of the methods disclosed herein, treatment with either Compound A or a pharmaceutically acceptable salt thereof, or Compound B or a pharmaceutically acceptable salt thereof, in combination with a RAF inhibitor, e.g., dabrafenib: induces prolonged cell stasis of tumor cells, e.g. GIST cells; induces killing of tumor cells, e.g., GIST cells; induces apoptosis of tumor cells, e.g., GIST cells; induces tumor cell eradication to the limit of detection, e.g., GIST cells; induces tumor regression, e.g., GIST tumor; reduces tumor weight or volume; e.g., GIST tumor; inhibits tumor regrowth, e.g., GIST tumor. In another aspect of any of the methods disclosed herein, treatment with either Compound A or a pharmaceutically acceptable salt thereof, or Compound B or a pharmaceutically acceptable salt thereof, in combination with a RAF inhibitor, e.g., dabrafenib: induces prolonged cell stasis of tumor cells, e.g. GIST cells; induces killing of tumor cells, e.g., GIST cells; induces apoptosis of tumor cells, e.g., GIST cells; induces tumor cell eradication to the limit of detection, e.g., GIST cells; induces tumor regression, e.g., GIST tumor; reduces tumor weight or volume; e.g., GIST tumor; inhibits tumor regrowth, e.g., GIST tumor in a drug resistant tumor, e.g. drug-resistant GIST. In another aspect of any of the methods disclosed herein, treatment with either Compound A or a pharmaceutically acceptable salt thereof, or Compound B or a pharmaceutically acceptable salt thereof, in combination with a RAF inhibitor, e.g., dabrafenib: eradicates a tumor to the limit of detection, in a patient being treated, e.g., a GIST patient. Methods for measuring or determining amounts of tumor cell stasis, tumor cell death, apoptosis of tumor cells, tumor regression, tumor weight or volume, tumor regrowth, growth of resistant tumor cells, and eradication of tumors are known in the art and include any methods described herein.

In particular embodiments, treatment with a combination of: Compound A or a pharmaceutically acceptable salt thereof, or Compound B or a pharmaceutically acceptable salt thereof; and a RAF inhibitor, e.g., dabrafenib, results in an increased amount of tumor cell stasis, killing of tumor cells or apoptosis of tumor cells, e.g., GIST cells, as compared to the amount of stasis, cell killing or apoptosis of tumor cells of the same type or same tumor type either untreated or treated with only a RAF inhibitor, e.g., dabrafenib, or with only a c-KIT inhibitor, e.g., imatinib, or with a combination of a RAF inhibitor, e.g., dabrafenib, with the c-KIT inhibitor imatinib. For example, cell stasis, cell killing or apoptosis may be increased by at least 10%, at least 20%, at least 30%, at least 40%, at least 50%, at least 60%, at least 70%, at least 80%, at least 90%, at least two-fold, at least three-fold, at least four-fold, at least five-fold, at least 10-fold, or at least 20-fold. In certain embodiments, amounts of apoptosis are determined by measuring caspase activity of tumor cells.

In particular embodiments, treatment with a combination of: Compound A or a pharmaceutically acceptable salt thereof, or Compound B or a pharmaceutically acceptable salt thereof; and a RAF inhibitor, e.g., dabrafenib, results in increased tumor regression or decreased tumor size or volume (e.g., a GIST), as compared to the size, e.g., weight or volume of a tumor of the same type or the same tumor either untreated or treated with only a RAF inhibitor, e.g., dabrafenib, or with only a c-KIT inhibitor, e.g., imatinib. For example, tumor weight or volume may be decreased by at least 10%, at least 20%, at least 30%, at least 40%, at least 50%, at least 60%, at least 70%, at least 80%, or at least 90%.

In particular embodiments, treatment with a combination of: Compound A or a pharmaceutically acceptable salt thereof, or Compound B or a pharmaceutically acceptable salt thereof; and a RAF inhibitor, e.g., dabrafenib, inhibits the amount of tumor growth or regrowth, e.g., GIST growth or regrowth, to a greater extent as compared to the amount of growth or regrowth the same type or the same tumor either untreated or treated with only a RAF inhibitor, e.g., dabrafenib, or with only a c-KIT inhibitor, e.g., imatinib. For example, tumor growth or regrowth may be inhibited by at least 10%, at least 20%, at least 30%, at least 40%, at least 50%, at least 60%, at least 70%, at least 80%, or at least 90%.

In particular embodiments, treatment with a combination of: Compound A or a pharmaceutically acceptable salt thereof, or Compound B or a pharmaceutically acceptable salt thereof; and a RAF inhibitor, e.g., dabrafenib, inhibits the growth of resistant tumor cells, e.g., resistant GIST cells, to a greater extent as compared to the amount of growth of resistant tumor cells of the same type or the same tumor either untreated or treated with only a RAF inhibitor, e.g., dabrafenib, or with only a c-KIT inhibitor, e.g., imatinib, or with a combination of a RAF inhibitor, e.g., dabrafenib and a c-KIT inhibitor, e.g., imatinib. For example, the amount of growth or number of resistant tumor cells may be inhibited by at least 10%, at least 20%, at least 30%, at least 40%, at least 50%, at least 60%, at least 70%, at least 80%, or at least 90%. In particular embodiments, resistant tumor cells are resistant to treatment with a c-KIT inhibitor, e.g., imatinib, and/or a RAF inhibitor, e.g., dabrafenib. In certain embodiments, the resistant tumor cells comprise a c-KIT secondary mutation. In certain embodiments, the c-KIT secondary mutation is a mutation of any of the following amino acid residues of c-KIT: V654, N655, T670, L783, D816, D820, N822, Y823, A829,and/or T847, including but not limited to any of the amino acid substitutions depicted in the accompanying figures. In particular embodiments, the resistant tumor cells comprise an activated MAPKAP kinase pathway, and in certain embodiments, they comprise a mutation in a mutation in a RAS gene, e.g., an N-RAS or K-RAS gene, a Fibloblast Growth Factor Receptor (FGFR) gene, and/or a Neurofibromin-1 (NF1) gene. In certain embodiments, the mutation is an N-RAS G12D mutation.

In particular embodiments, treatment with a combination of: either Compound A or a pharmaceutically acceptable salt thereof, or Compound B or a pharmaceutically acceptable salt thereof; in combination with a RAF inhibitor, e.g., dabrafenib, results in eradication of a tumor to the limit of detection, e.g., a GIST. In particular embodiments, eradication of a tumor means there is no longer any detectable tumor in the patient to the limit of detection. In particular embodiments, there is no detectable tumor in the patient for at least six months, at least one year, at least two years, at least five years, or at least 10 years following eradication of the tumor, e.g., GIST, by a combination therapy disclosed herein. Tumor eradication may be determined by photon emission tomography (PET), CT scans, absence of circulating cell free DNA (cfDNA) containing a c-KIT mutation, absence of circulating tumor cells (CTCs) present in the vasculature of a subject, or absence of a cancer cell biomarker within the circulating blood vasculature of a subject.

The present disclosure describes combination therapies that involve the administration of either Compound A or a pharmaceutically acceptable salt thereof, or Compound B or a pharmaceutically acceptable salt thereof, and one or more MAPKAP kinase inhibitors, e.g., a MEK inhibitor, ERK inhibitor, or RAF inhibitor. The combination therapies described herein can be used by themselves, or in further combination with one or more additional therapeutic agents (e.g., one or more additional therapeutic agents described below). For example, either Compound A or a pharmaceutically acceptable salt thereof, or Compound B or a pharmaceutically acceptable salt thereof, and a MEK inhibitor, can be administered together with a cancer targeted therapeutic agent, a cancer-targeted biological, an immune checkpoint inhibitor, or a chemotherapeutic agent. In another embodiment Compound A or Compound B and a MEK inhibitor are administered without any other therapeutic agent. The therapeutic agents can be administered together with or sequentially with another therapeutic agent described herein in a combination therapy.

Combination therapy can be achieved by administering two or more therapeutic agents, each of which is formulated and administered separately, or by administering two or more therapeutic agents in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The one or more additional therapeutic agents that may be administered according to the present disclosure include, but are not limited to, cytotoxic agents, cisplatin, doxorubicin, etoposide, irinotecan, topotecan, paclitaxel, docetaxel, the epothilones, tamoxifen, 5-fluorouracil, methotrexate, temozolomide, cyclophosphamide, lonafarib, tipifarnib, 4- ((5- ((4-(3-chlorophenyl)-3-oxopiperazin-1-yl) methyl)-1H-imidazol-1-yl) methyl) benzonitrile hydrochloride, (R)-1- ((1H-imidazol-5-yl) methyl)-3-benzyl-4-(thiophen-2-ylsulfonyl)-2,3,4,5-tetrahydro-1H-benzo diazepine-7-carbonitrile, cetuximab, imatinib, interferon alfa-2b, pegylated interferon alfa-2b, aromatase combinations, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, leucovorin, oxaliplatin, pentostatine, vinblastine, vincristine, vindesine, bleomycin, dactinomycin, daunorubicin, epirubicin, idarubicin, mithramycin, deoxycoformycin, mitomycin-C, L-asparaginase, teniposide 17α-ethinyl estradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, 17α-hydroxyprogesterone, aminoglutethimide, estramustine, medroxyprogesterone acetate, leuprolide acetate, flutamide, toremifene citrate, goserelin acetate, carboplatin, hydroxyurea, amsacrine, procarbazine, mitotane, mitoxantrone, levamisole, vinorelbine, anastrazole, letrozole, capecitabine, raloxifene, droloxafine, hexamethylmelamine, bevacizumab, trastuzumab, tositumomab, bortezomib, ibritumomab tiuxetan, arsenic trioxide, porfimer sodium, cetuximab, thioTEPA, altretamine, fulvestrant, exemestane, rituximab, alemtuzumab, dexamethasone, bicalutamide, chlorambucil, and valrubicin.

The one or more additional therapeutic agents that can be administered may include, without limitation, an AKT inhibitor, alkylating agent, all-trans retinoic acid, antiandrogen, azacitidine, BCL2 inhibitor, BCL-XL inhibitor, BCR-ABL inhibitor, BTK inhibitor, BTK/LCK/LYN inhibitor, CDK1/2/4/6/7/9 inhibitor, CDK4/6 inhibitor, CDK9inhibitor, CBP/p300 inhibitor, EGFR inhibitor, endothelin receptor antagonist, RAF inhibitor, MEK inhibitor, ERK inhibitor, farnesyltransferase inhibitor, FLT3 inhibitor, glucocorticoid receptor agonist, HDM2 inhibitor, histone deacetylase inhibitor, IKKβ inhibitor, immunomodulatory drug (IMiD), ingenol, ITK inhibitor, JAK1/JAK2/JAK3/TYK2 inhibitor, MTOR inhibitor, PI3 kinase inhibitor, dual PI3 kinase/MTOR inhibitor, proteasome inhibitor, protein kinase C agonist, SUV39H1 inhibitor, TRAIL, VEGFR2 inhibitor, Wnt/β-catenin signaling inhibitor, decitabine, and anti-CD20 monoclonal antibody.

In certain embodiments, the additional therapeutic agent is an immunomodulatory agentis selected from the group consisting of CTLA4 inhibitors such as, but not limited to ipilimumab and tremelimumab; PD1 inhibitors such as, but not limited to pembrolizumab, and nivolumab; PDL1 inhibitors such as, but not limited to atezolizumab (formerly MPDL3280A), durvalumab (formerly MEDI4736), avelumab, PDR001; 4 1BB or 4 1BB ligand inhibitors such as, but not limited to urelumab and PF-05082566; OX40 ligand agonists such as, but not limited to MEDI6469; GITR agents such as, but not limited to TRX518; CD27 inhibitors such as, but not limited to varlilumab; TNFRSF25 or TL1A inhibitors; CD40 agonists such as, but not limited to CP-870893; HVEM or LIGHT or LTA or BTLA or CD160 inhibitors; LAG3 inhibitors such as, but not limited to BMS-986016;TIM3 inhibitors; Siglecs inhibitors; ICOS or ICOS ligand agonists; B7 H3 inhibitors such as, but not limited to MGA271; B7 H4 inhibitors; VISTA inhibitors; HHLA2 or TMIGD2inhibitors; inhibitors of Butyrophilins, including BTNL2 inhibitors; CD244 or CD48inhibitors; inhibitors of TIGIT and PVR family members; KIRs inhibitors such as, but not limited to lirilumab; inhibitors of ILTs and LIRs; NKG2D and NKG2A inhibitors such as, but not limited to IPH2201; inhibitors of MICA and MICB; CD244 inhibitors; CSFIR inhibitors such as, but not limited to emactuzumab, cabiralizumab, pexidartinib, ARRY382,BLZ945; IDO inhibitors such as, but not limited to INCB024360; thalidomide, lenalidomide, TGFß inhibitors such as, but not limited to galunisertib; adenosine or CD39 or CD73inhibitors; CXCR4 or CXCL12 inhibitors such as, but not limited to ulocuplumab and (3S,6S,9S,12R,17R,20S,23S,26S,29S,34aS)-N-((S)-1-amino-5-guanidino-1-oxopentan-2-yl)-26,29-bis (4-aminobutyl)-17-((S)-2- ((S)-2- ((S)-2-(4-fluorobenzamido)-5-guanidinopentanamido)-5-guanidinopentanamido)-3-(naphthalen-2-yl) propanamido)-6-(3-guanidinopropyl)-3,20-bis (4-hydroxybenzyl)-1,4,7,10,18,21,24,27,30-nonaoxo-9,23-bis (3-ureidopropyl) triacontahydro-1H, 16H-pyrrolo [2,1-p][1,2] dithia [5,8,11,14,17,20,23,26,29] nonaazacyclodotriacontine-12-carboxamide BKT140;phosphatidylserine inhibitors such as, but not limited to bavituximab; SIRPA or CD47inhibitors such as, but not limited to CC-90002; VEGF inhibitors such as, but not limited to bevacizumab; and neuropilin inhibitors such as, but not limited to MNRP1685A.

### Pharmaceutical Compositions

Aspects of the present disclosure are directed to methods of treatment

involving the administration of a combination of compounds disclosed herein, or one or more pharmaceutical compositions comprising such compounds and a pharmaceutically acceptable diluent, excipient or carrier. In particular embodiments, the methods disclosed herein involve administering a first pharmaceutical composition comprising either Compound A or a pharmaceutically acceptable salt thereof thereof and a pharmaceutically acceptable diluent, excipient or carrier, and a second pharmaceutical composition comprising a MEK inhibitor, e.g., trametinib, a ERK inhibitor, e.g. ulixertinib, a RAF inhibitor, e.g. LY3009120, or pharmaceutically acceptable salts thereof, and a pharmaceutically acceptable diluent, excipient or carrier. In particular embodiments, the methods disclosed herein involve administering a first pharmaceutical composition comprising Compound B or a pharmaceutically acceptable salt thereof and a pharmaceutically acceptable diluent, excipient or carrier, and a second pharmaceutical composition comprising a MEK inhibitor, e.g., trametinib, a ERK inhibitor, e.g. ulixertinib, a RAF inhibitor, e.g. LY3009120, or pharmaceutically acceptable salts thereof, and a pharmaceutically acceptable diluent, excipient or carrier. In particular embodiments, the methods disclosed herein involve administering a pharmaceutical composition comprising Compound A or a pharmaceutically acceptable salt thereof, a MEK inhibitor, e.g., trametinib, and a pharmaceutically acceptable diluent, excipient or carrier. In particular embodiments, the methods disclosed herein involve administering a pharmaceutical composition comprising Compound B or a pharmaceutically acceptable salt thereof, a MEK inhibitor, e.g., trametinib, and a pharmaceutically acceptable diluent, excipient or carrier.

In particular embodiments, the methods disclosed herein involve administering a pharmaceutical composition comprising Compound A or a pharmaceutically acceptable salt thereof, a ERK inhibitor, e.g., ulixertinib, and a pharmaceutically acceptable diluent, excipient or carrier. In particular embodiments, the methods disclosed herein involve administering a pharmaceutical composition comprising Compound B or a pharmaceutically acceptable salt thereof, a ERK inhibitor, e.g., ulixertinib, and a pharmaceutically acceptable diluent, excipient or carrier.

In particular embodiments, the methods disclosed herein involve

administering a pharmaceutical composition comprising Compound A or a pharmaceutically acceptable salt thereof, a RAF inhibitor, e.g., LY3009120, and a pharmaceutically acceptable diluent, excipient or carrier. In particular embodiments, the methods disclosed herein involve administering a pharmaceutical composition comprising Compound B or a pharmaceutically acceptable salt thereof, a RAF inhibitor, e.g., LY3001290, and a pharmaceutically acceptable diluent, excipient or carrier.

In using the pharmaceutical compositions of the compounds described herein, pharmaceutically acceptable carriers can be either solid or liquid. Solid forms include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa, which is hereby incorporated by reference in its entirety.

Liquid form preparations include solutions, suspensions and emulsions. For example, water or water-propylene glycol solutions for parenteral injection or addition of sweeteners and opacifiers for oral solutions, suspensions and emulsions. Liquid form preparations may also include solutions for intranasal administration.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Aerosol preparations suitable for inhalation may also be used. These preparations may include solutions and solids in powder form, which may be in combination with a pharmaceutically acceptable carrier, such as an inert compressed gas, e.g., nitrogen.

Also contemplated for use are solid form preparations that are intended to be converted, shortly before use, to liquid form preparations for either oral or parenteral administration. Such liquid forms include solutions, suspensions and emulsions.

### Dosage

In some embodiments where Compound A or Compound B (or

pharmaceutically acceptable salts thereof) is used in combination with a MEK inhibitor (e.g., trametinib) for a treatment protocol, the two therapeutics may be administered together or in a “dual-regimen” wherein the two therapeutics are dosed and administered separately. When the Compound A or B (or pharmaceutically acceptable salts thereof) and the MEK inhibitor are dosed separately, the typical dosage of Compound A or Compound B (or pharmaceutically acceptable salts thereof) administered to the subject in need of the treatment is typically from about 5 mg per day to about 5000 mg per day and, in other embodiments, from about 50 mg per day to about 1000 mg per day. Other dosages may be from about 10 mmol up to about 250 mmol per day, from about 20 mmol to about 70 mmol per day or even from about 30 mmol to about 60 mmol per day. Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. A typical recommended daily dosage regimen for oral administration can range from about 1 mg/day to about 500 mg/day or 1 mg/day to 200 mg/day, in a single dose, or in two to four divided doses. In one embodiment, the typical daily dose regimen is 150 mg.

In certain embodiments, the dosage of MEK inhibitors is consistent with

previously disclosed dosages and/or dosages approved for use by the Food and Drug Administration. In other embodiments, the dosage of MEK inhibitor is less than previously approved dosages, e.g., about 20%, about 50% or about 80% of an approved dosage. In certain embodiments, the dosage of trametinib is about 0.5 mg to 20 mg orally daily, e.g., about 1 mg daily or about 2 mg daily. In certain embodiments, the dosage of cobimetinib is about 10 mg to 200 mg daily, e.g., about 30 mg or about 60 mg daily. In certain embodiments, the dosage of binimetinib is about 10 mg to about 200 mg twice daily, e.g., about 25 mg or about 45 mg twice daily. In certain embodiments, the dosage of selumetinib is about 10 mg to 200 mg daily, e.g., about 30 mg or about 75 mg twice daily.

The amount and frequency of administration of the compounds described herein and/or the pharmaceutically acceptable salts thereof, and other therapeutic agents, will be regulated according to the judgment of the attending clinician considering such factors as age, condition and size of the patient as well as severity of the symptoms being treated.

Compounds of the present disclosure (e.g., Compound A or Compound B (and pharmaceutically acceptable salts thereof), MEK inhibitors, and other therapeutic agents) may be administered by any suitable route. The compounds can be administrated orally (e.g., dietary) in capsules, suspensions, tablets, pills, dragees, liquids, gels, syrups, slurries, and the like. Methods for encapsulating compositions (such as in a coating of hard gelatin or cyclodextran) are known in the art (Baker, et al., “Controlled Release of Biological Active Agents”, John Wiley and Sons, 1986, which is hereby incorporated by reference in its entirety). The compounds can be administered to the subject in conjunction with an acceptable pharmaceutical carrier as part of a pharmaceutical composition. The formulation of the pharmaceutical composition will vary according to the route of administration selected. Suitable pharmaceutical carriers may contain inert ingredients which do not interact with the compound. The carriers are biocompatible, i.e., non-toxic, non-inflammatory, non-immunogenic and devoid of other undesired reactions at the administration site.

Illustrative pharmaceutical compositions are tablets and gelatin capsules comprising a compound described herein and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

If formulated as a fixed dose, such combination products employ the compounds described herein within the dosage range described herein, or as known to those skilled in the art.

Since the compounds described herein (e.g., Compounds A and B and MAPKAP kinase inhibitors including MEK inhibitors) are intended for use in pharmaceutical compositions a skilled artisan will understand that they can be provided in substantially pure forms for example, at least 60% pure, at least 75% pure, at least 85% pure, and at least 98% pure (w/w). The pharmaceutical preparation may be in a unit dosage form. In such form, the preparation is subdivided into suitably sized unit doses containing appropriate quantities of compounds A or B, e.g., an effective amount to achieve the desired purpose as described herein.

### EXAMPLES

It is found that treatment of c-KIT-mediated tumor cells with a combination of either Compound A or a pharmaceutically acceptable salt thereof in combination with a MEK inhibitor or an ERK inhibitor, or a RAF inhibitor unexpectedly and synergistically induces apoptosis of the tumor cells. In addition, this combination therapy prevents growth of tumor cells, including tumor cells having a secondary mutation conferring resistance to other c-KIT inhibitors and/or MEK, ERK or RAF inhibitors. Furthermore, the combination therapy disclosed herein appeared to have a prolonged effect on tumor cell stasis, as opposed to rapid tumor regrowth in the absence of drug combination treatment. Furthermore, the combination therapy disclosed herein appeared to have a cytotoxic effect on tumor cells, as opposed to merely a cytostatic effect. Furthermore, the combination therapy disclosed herein appeared to eradicate GIST tumor cells to the limit of detection, with no tumor cell colony outgrowth after removal of combination therapy including drug-free recovery periods of up to 21 days. Characterization of this unexpected finding was undertaken in biochemical assays and cellular assays, including those described herein.

The disclosure is thus further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Combination Treatment of Compound A with Trametinib Induces Apoptosis in GIST-T1, GIST-T1/D816E Imatinib-Resistant Cells and GIST-T1/T670I Imatinib Resistant Cells

A study was performed which demonstrates that combination treatment with Compound A and trametinib induces apoptosis in GIST-T1 (57 bp exon 11 deletion) imatinib sensitive cells, GIST-T1/D816E imatinib resistant cells and GIST-T1/T6701) imatinib resistant cells. Assays were conducted in 96 well plates with 10,000 GIST-T1, GIST-T1/D816E or GIST-T1/T670I cells seeded per well. Cells were treated with vehicle control, Compound A, trametinib, or combinations thereof at varying concentrations, and the cells were allowed to grow for 24 and 48 hours in the presence of the drug treatments. Apoptosis was assessed by measuring Caspase 3/7 activity.

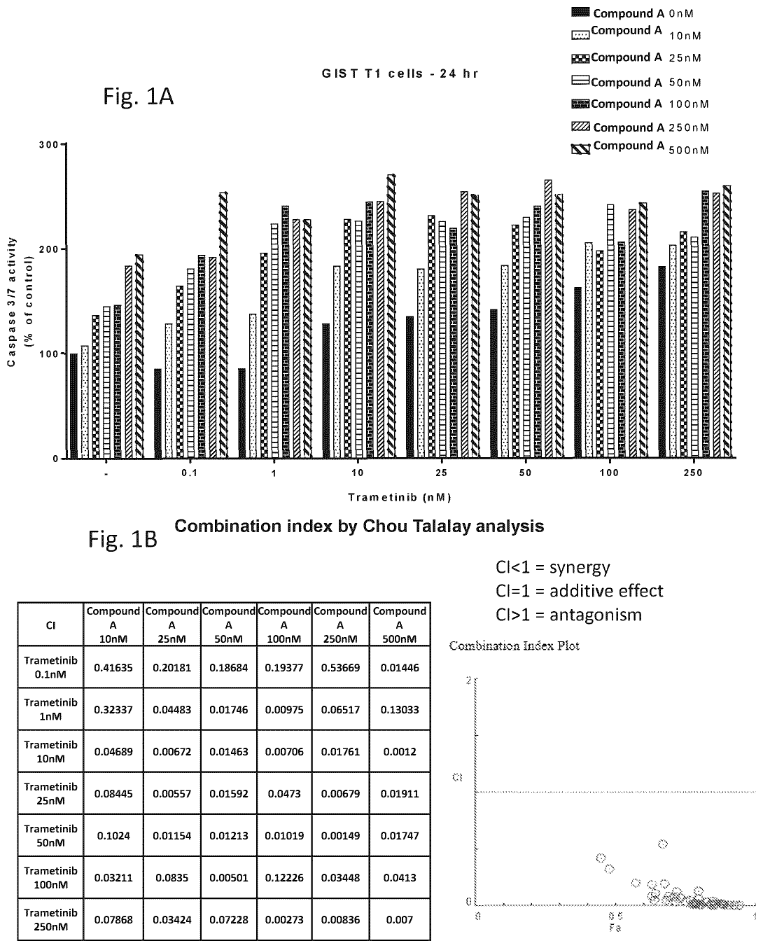

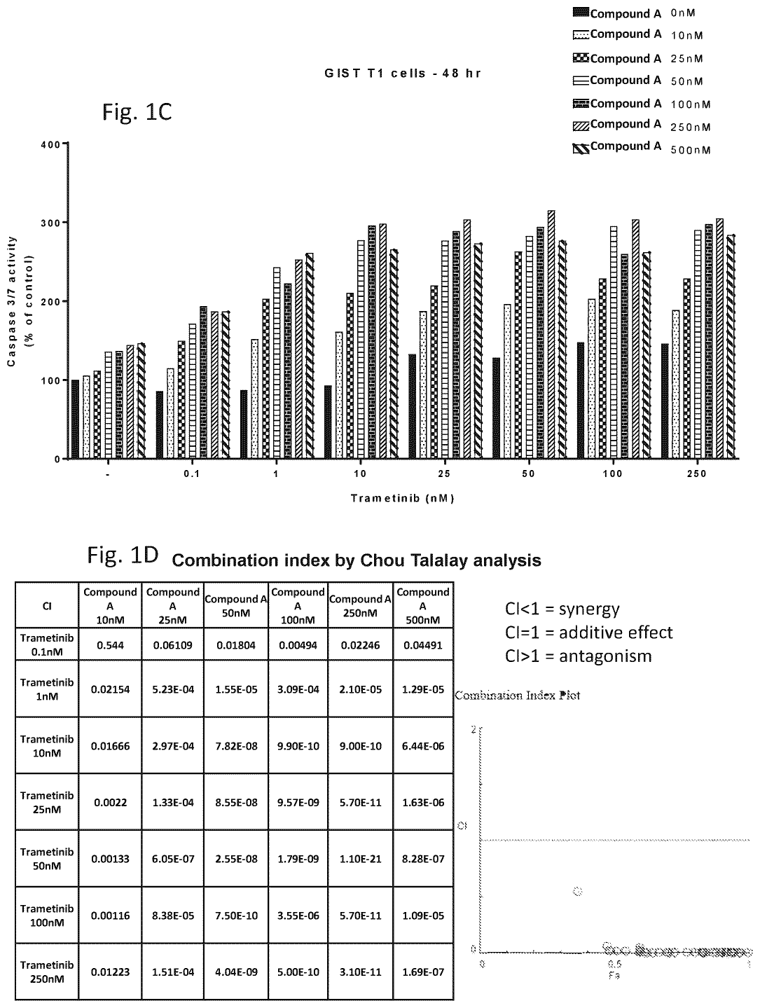

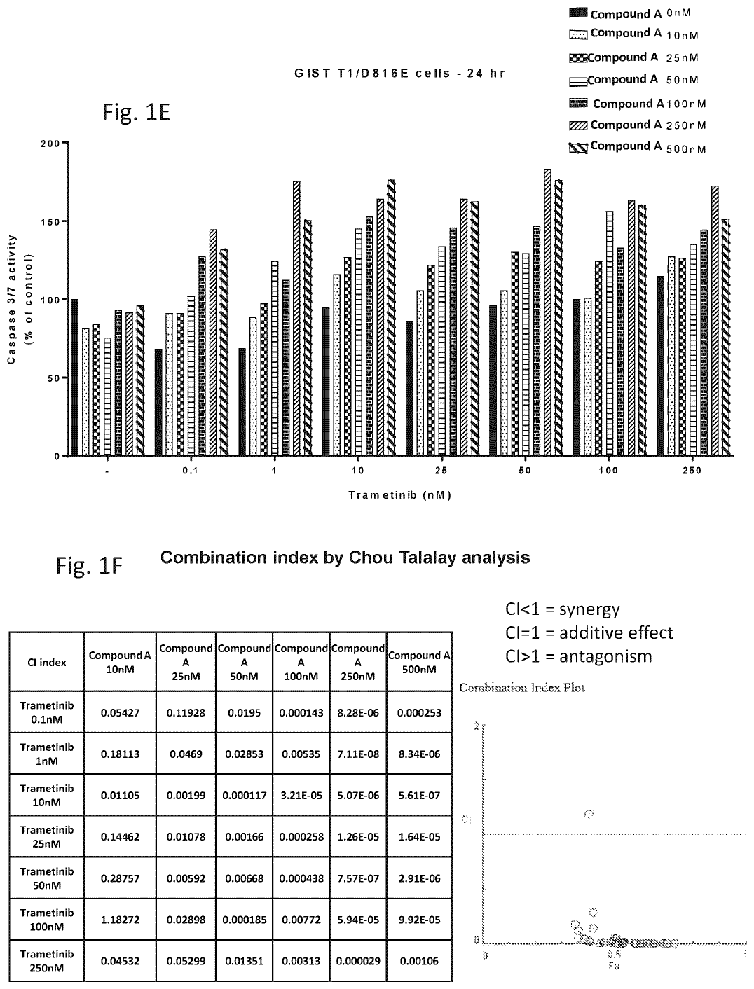

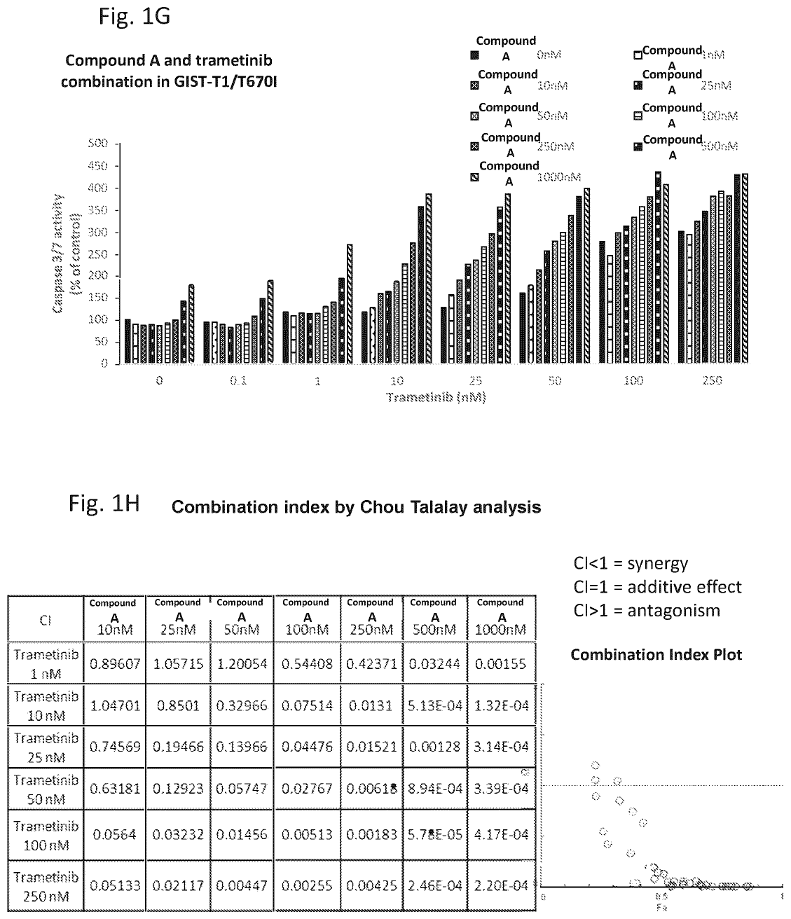

FIGS. 1A and 1C are graphical representations showing the relative percentage of caspase activity (compared to vehicle control set at 100%) determined for various treatments of GIST-T1 cells. FIGS. 1B and 1D are matrix synergy charts and combination index plots based on the combination index (CI) method described by Chou and Talalay (1984) and the computer software of Chou and Martin (2005). CI<1 indicates synergism, CI=1 indicates additive effect, and CI>1 indicates antagonism. Combination treatments for 24 hours (FIGS. 1A, B) and 48 hours (FIGS. 1C, D) with Compound Aand trametinib showed strong synergy for inducing apoptosis in GIST-T1 cells.

FIG. 1E is a graphical representation showing caspase activity from various treatments of GIST-T1/D816E imatinib resistant cells. FIG. 1F is a matrix synergy chart and combination index plot based on the combination index (CI) method described by Chou and Talalay (1984) and the computer software of Chou and Martin (2005). CI<1 indicates synergism, CI=1 indicates additive effect, and CI>1 indicates antagonism. Combination treatments for 24 hours with Compound A and trametinib showed strong synergy for inducing apoptosis of GIST-T1/D816E imatinib resistant cells.

FIG. 1G is a graphical representation showing caspase activity from various treatments of GIST-T1/T670I imatinib resistant cells. FIG. 1H is a matrix synergy chart and combination index plot based on the combination index (CI) method described by Chou and Talalay (1984) and the computer software of Chou and Martin (2005). CI<1 indicates synergism, CI=1 indicates additive effect, and CI>1 indicates antagonism. Combination treatments for 24 hours with Compound A and trametinib showed strong synergy for inducing apoptosis of GIST-T1/T670I imatinib resistant cells.

### Example 2

### Combination Treatment of Compound B with Trametinib Induces Apoptosis in GIST-T1, GIST-T1/D816E Imatinib-Resistant Cells and GIST-T1/T670I Imatinib Resistant Cells

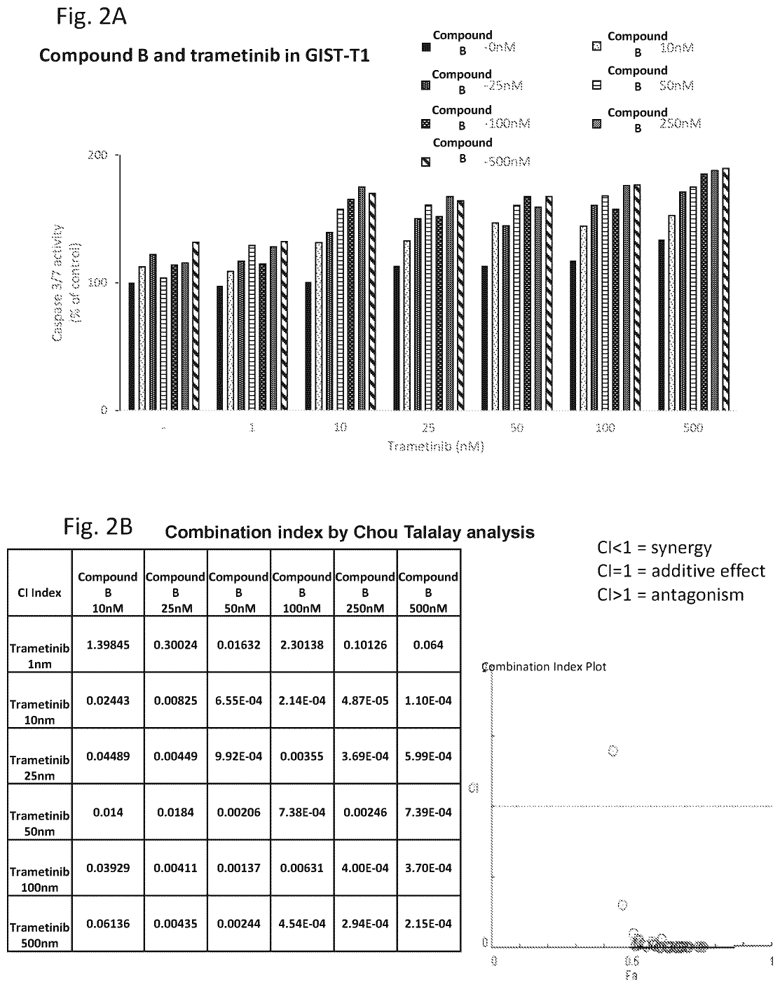

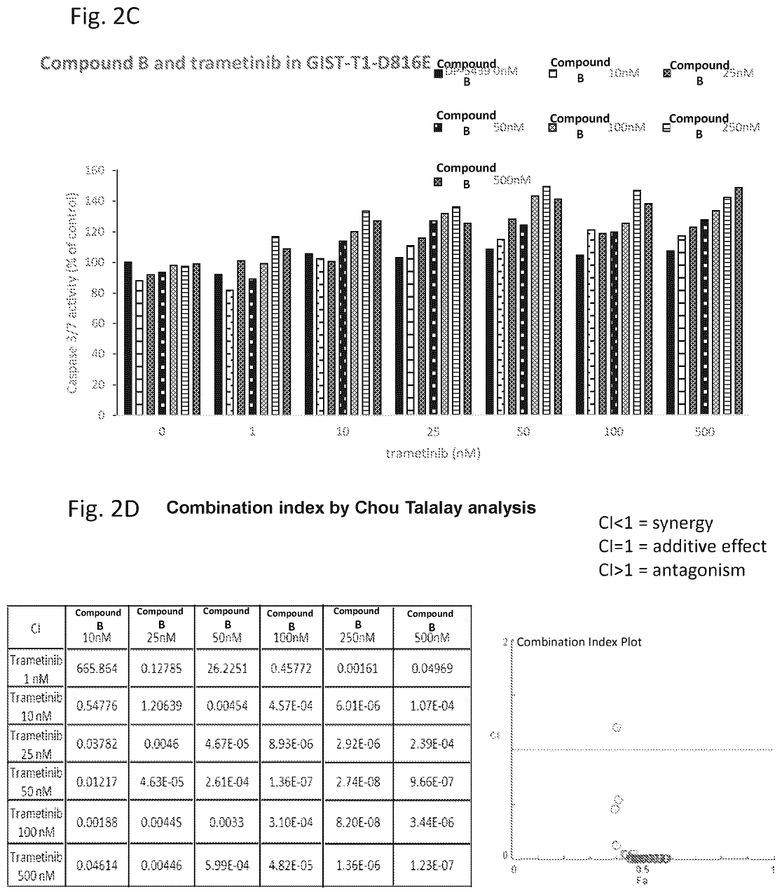

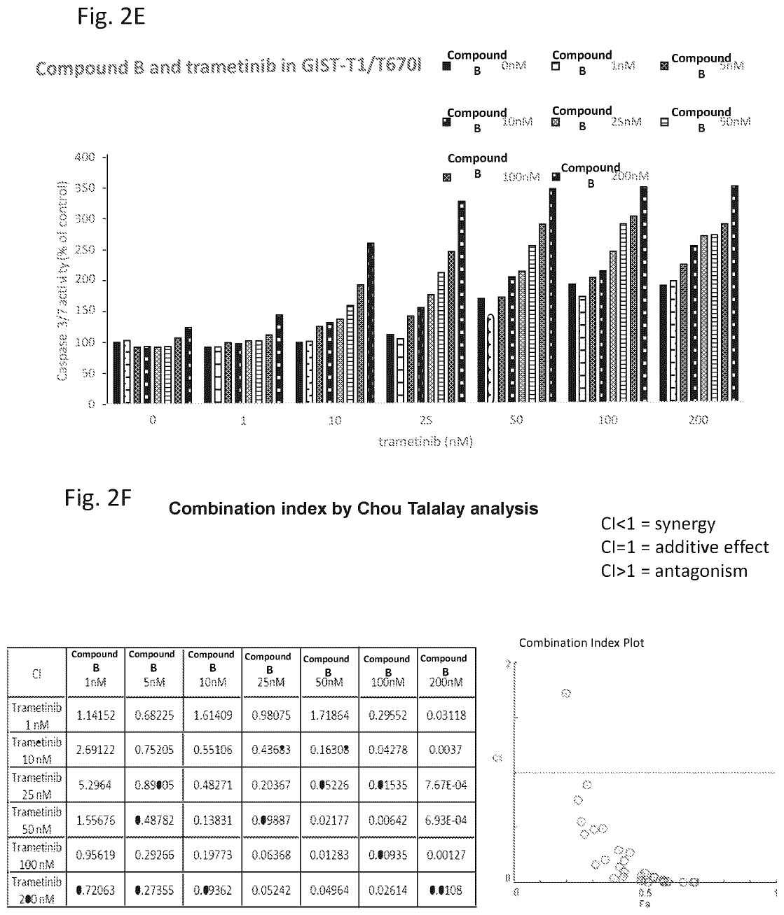

FIG. 2A is a graphical representations showing the relative percentage of caspase activity (compared to vehicle control set at 100%) determined for various treatments of GIST-T1 cells. FIG. 2B is a matrix synergy charts and combination index plots as described in example 1 Combination treatments for 24 hours (FIGS. 2A, B) with Compound B and trametinib showed strong synergy for inducing apoptosis in GIST-T1 cells.

FIG. 2C is a graphical representation showing caspase activity from various treatments of GIST-T1/D816E imatinib resistant cells. FIG. 2D is a matrix synergy chart and combination index plot. Combination treatments for 24 hours with Compound B and trametinib showed strong synergy for inducing apoptosis of GIST-T1/D816E imatinib resistant cells.

FIG. 2E is a graphical representation showing caspase activity from various treatments of GIST-T1/T670I imatinib resistant cells. FIG. 2F is a matrix synergy chart and combination index plot. Combination treatments for 24 hours with Compound B and trametinib showed strong synergy for inducing apoptosis of GIST-T1/T670I imatinib resistant cells.

### Example 3

### Combination Treatment of Compound A with Binimetinib Induces Apoptosis in GIST-T1 and GIST-T1/D816E Imatinib-Resistant Cells and GIST-T1/T670I Imatinib Resistant Cells

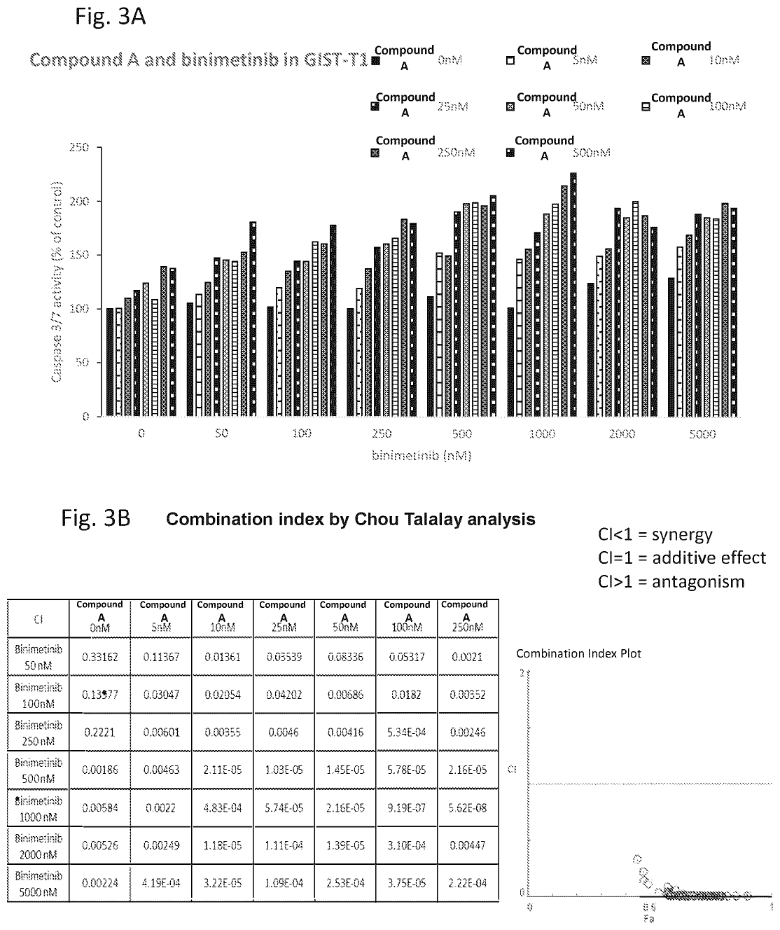

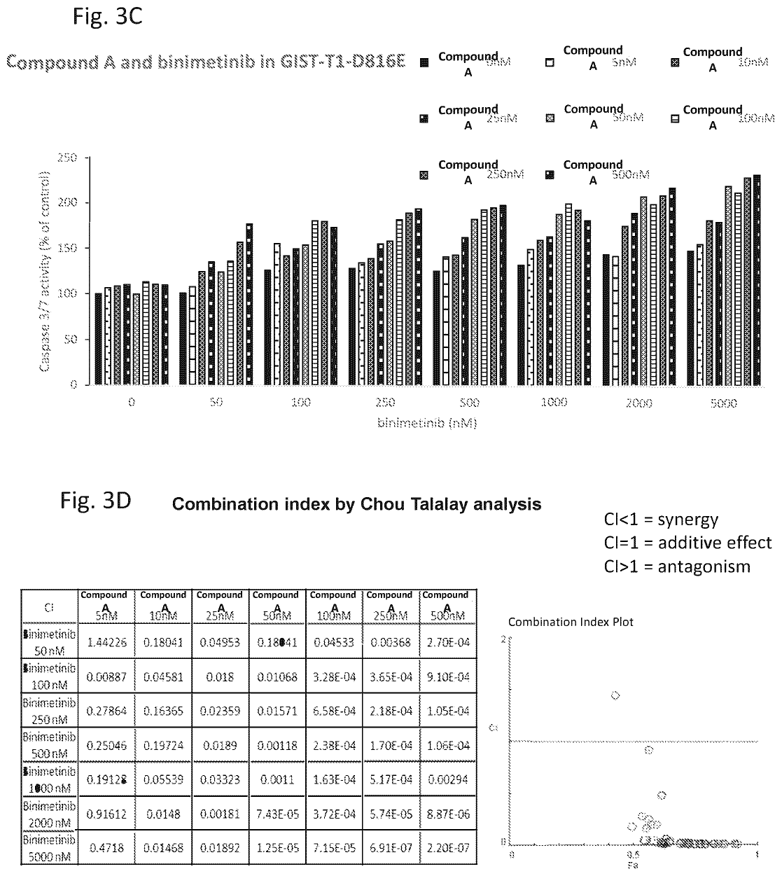

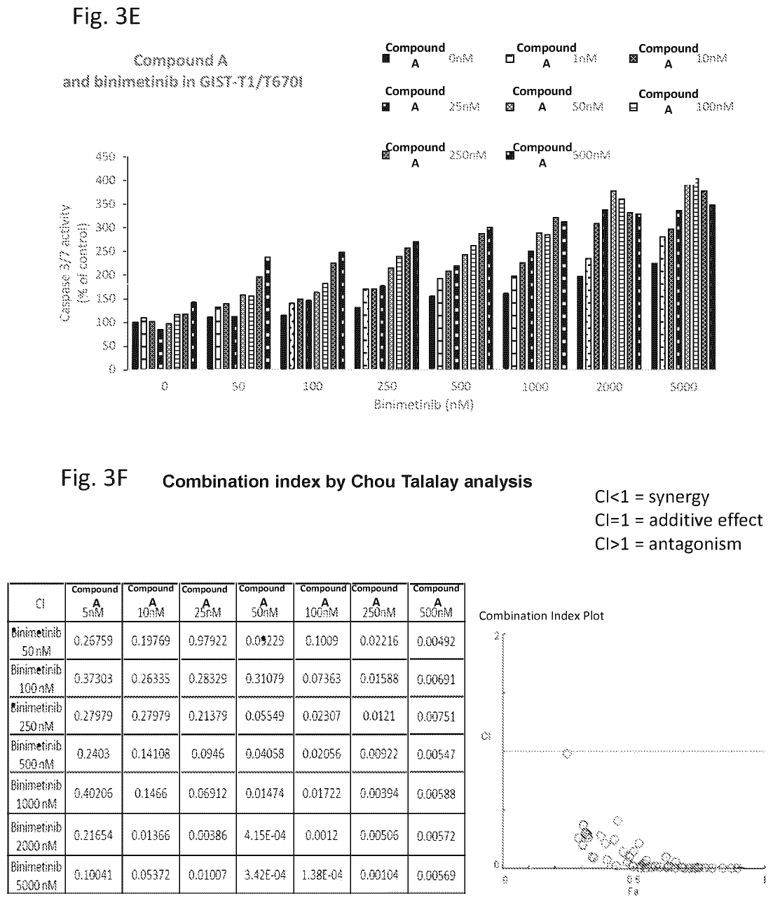

FIG. 3A is graphical representations showing the relative percentage of caspase activity (compared to vehicle control set at 100%) determined for various treatments of GIST-T1 cells. FIG. 3B shows matrix synergy charts and combination index plots based on the combination index (CI) method as described in example 1. Combination treatments for 24 hours (FIGS. 3A, 3B) with Compound A and binimetinib showed strong synergy for inducing apoptosis in GIST-T1 cells.

FIG. 3C is a graphical representation showing caspase activity from various treatments of GIST-T1/D816E imatinib resistant cells. FIG. 3D is a matrix synergy chart and combination index plot based on the combination index (CI) as described in example 1. Combination treatments for 24 hours with Compound A and binimetinib showed strong synergy for inducing apoptosis of GIST-T1/D816E imatinib resistant cells.

FIG. 3E is a graphical representation showing caspase activity from various treatments of GIST-T1/T670I imatinib resistant cells. FIG. 3F is a matrix synergy chart and combination index plot. Combination treatments for 24 hours with Compound A and binimetinib showed strong synergy for inducing apoptosis of GIST-T1/T670I imatinib resistant cells.

### Example 4

### Combination Treatment of Compound B with Binimetinib Induces Apoptosis in GIST-T1, GIST-T1/D816E Imatinib-Resistant Cells and GIST-T1/T670I Imatinib Resistant Cells

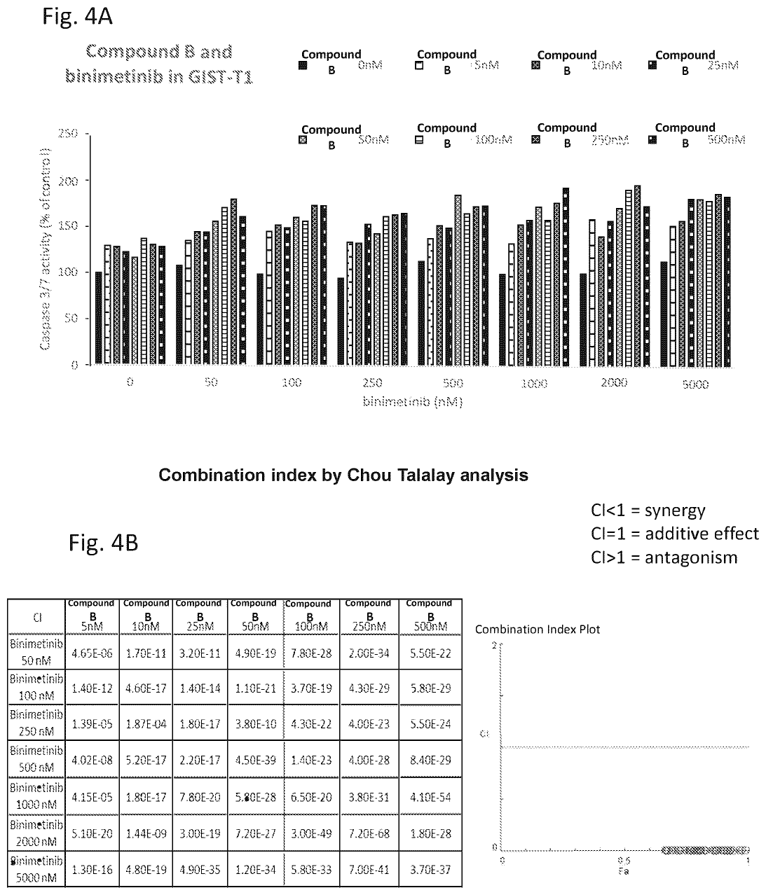

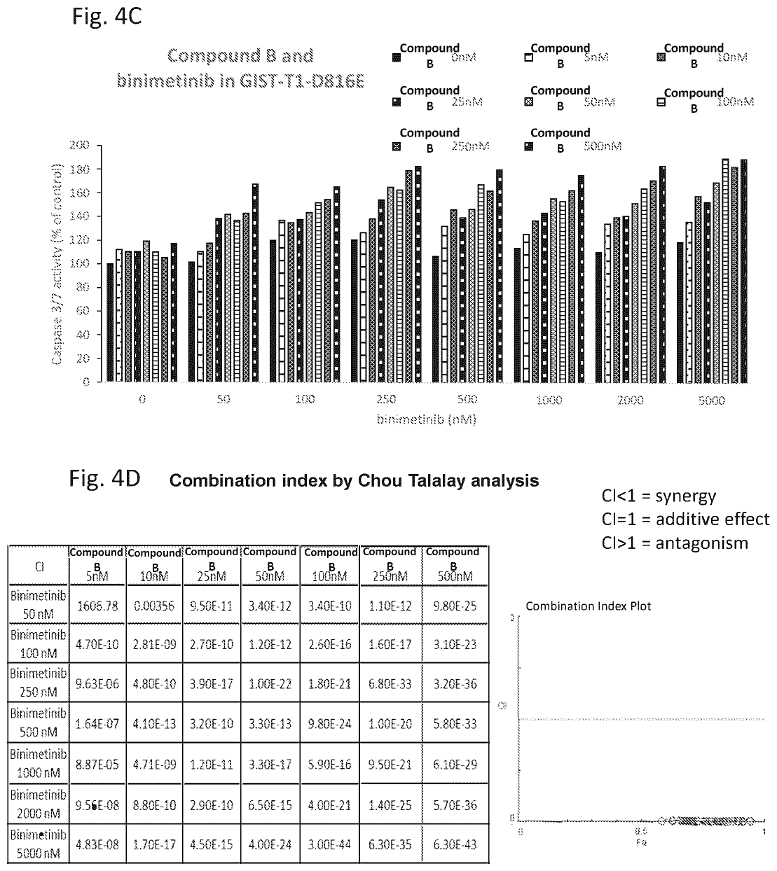

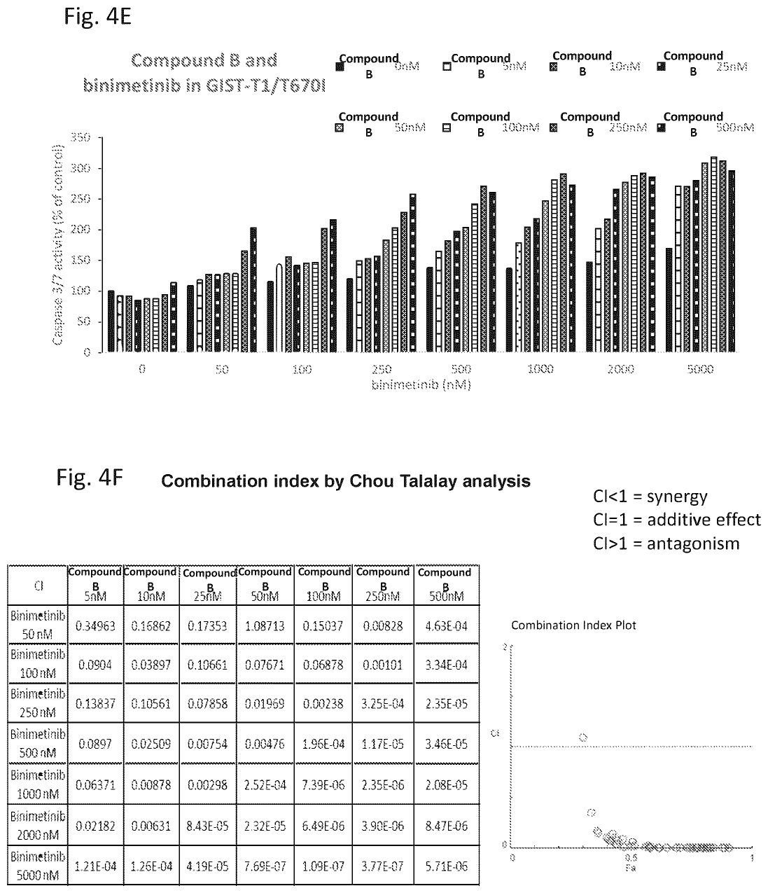

FIG. 4A is a graphical representations showing the relative percentage of caspase activity (compared to vehicle control set at 100%) determined for various treatments of GIST-T1 cells. FIG. 4B is a matrix synergy charts and combination index plots based on the combination index (CI) method described by Chou and Talalay (1984). Combination treatments for 24 hours (FIGS. 4A, B) with Compound B and binimetinib showed strong synergy for inducing apoptosis in GIST-T1 cells.

FIG. 4C is a graphical representation showing caspase activity from various treatments of GIST-T1/D816E imatinib resistant cells. FIG. 4D is a matrix synergy chart and combination index plot. Combination treatments for 24 hours with Compound B and binimetinib showed strong synergy for inducing apoptosis of GIST-T1/D816E imatinib resistant cells.

FIG. 4E is a graphical representation showing caspase activity from various treatments of GIST-T1/T670I imatinib resistant cells. FIG. 4F is a matrix synergy chart and combination index plot. Combination treatments for 24 hours with Compound B and binimetinib showed strong synergy for inducing apoptosis of GIST-T1/T670I imatinib resistant cells.

### Example 5

### Combination Treatment of Compound A with Cobimetinib Induces Apoptosis in GIST-T1 Imatinib Sensitive Cells, GIST-T1/D816E Imatinib-Resistant Cells and GIST-T1/T670I Imatinib Resistant Cells

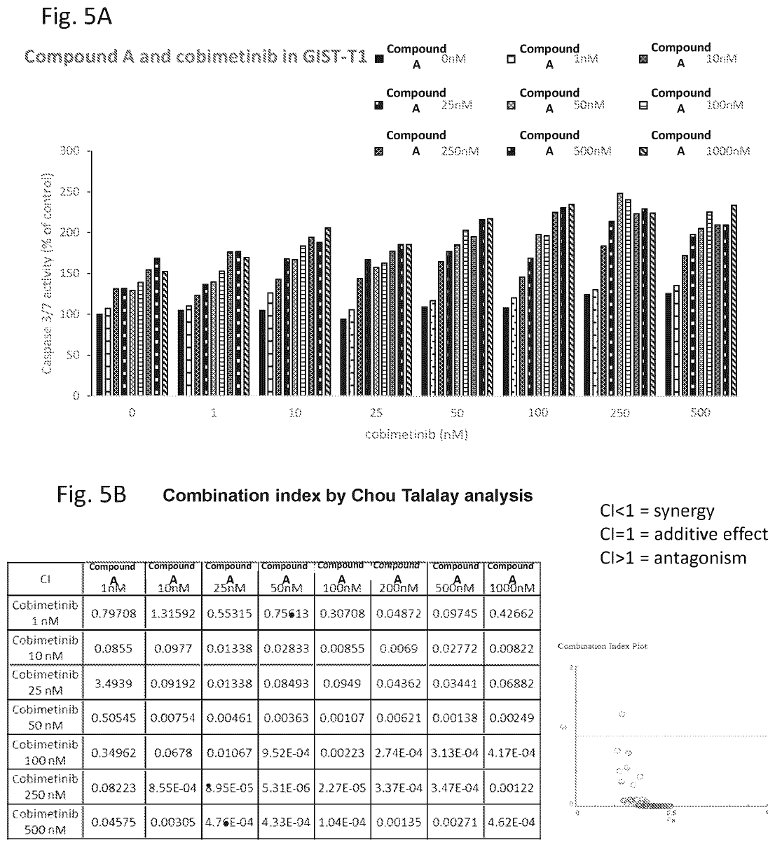

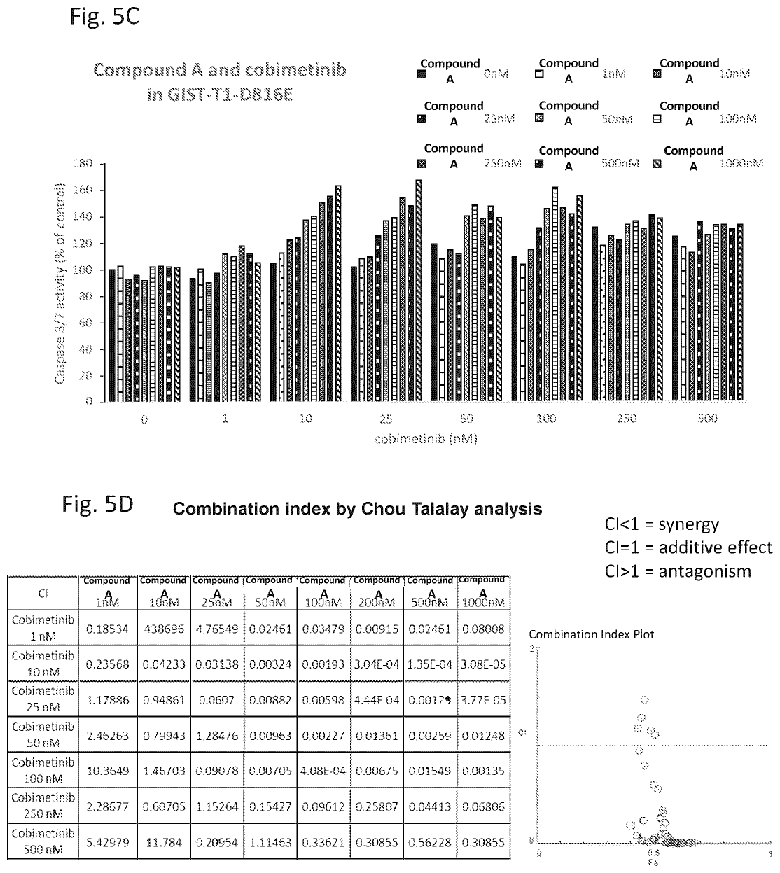

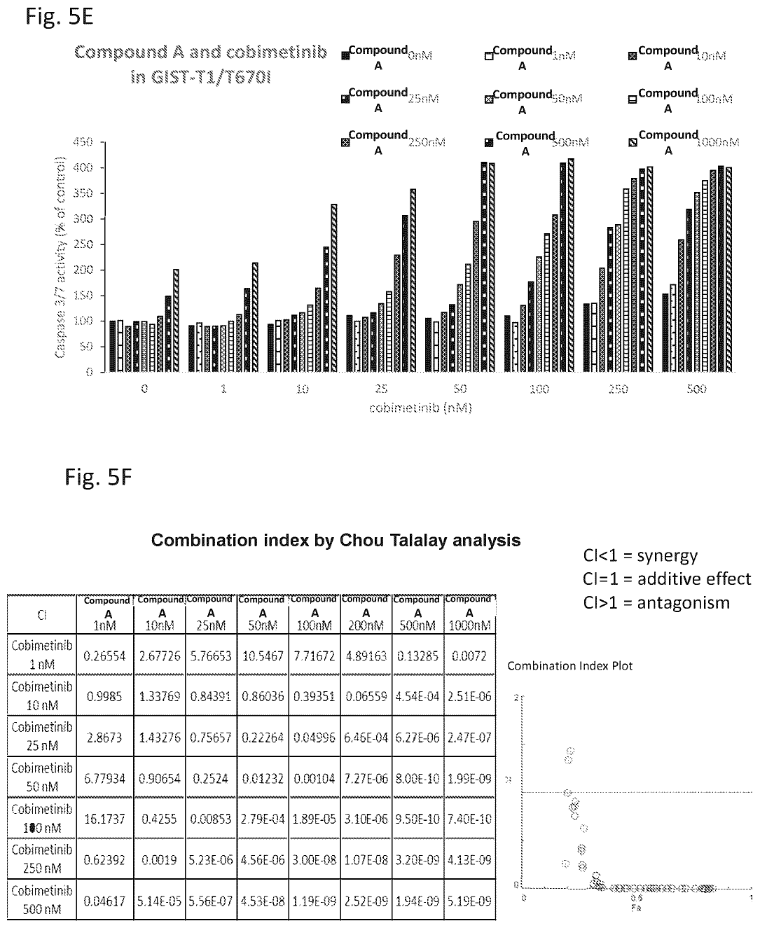

FIG. 5A is graphical representations showing the relative percentage of caspase activity (compared to vehicle control set at 100%) determined for various treatments of GIST-T1 cells. FIG. 5B shows matrix synergy charts and combination index plots based on the combination index (CI) method as described in example 1. Combination treatments for 24 hours (FIGS. 5A, 5B) with Compound A and cobimetinib showed strong synergy for inducing apoptosis in GIST-T1 cells.

FIG. 5C is a graphical representation showing caspase activity from various treatments of GIST-T1/D816E imatinib resistant cells. FIG. 5D is a matrix synergy chart and combination index plot based on the combination index (CI) as described in example 1. Combination treatments for 24 hours with Compound A and cobimetinib showed strong synergy for inducing apoptosis of GIST-T1/D816E imatinib resistant cells.

FIG. 5E is a graphical representation showing caspase activity from various treatments of GIST-T1/T670I imatinib resistant cells. FIG. 5F is a matrix synergy chart and combination index plot based on the combination index (CI) as described in example 1. Combination treatments for 24 hours with Compound A and cobimetinib showed strong synergy for inducing apoptosis of GIST-T1/T670I imatinib resistant cells.

### Example 6

### Combination Treatment of Compound B with Cobimetinib Induces Apoptosis in GIST-T1, GIST-T1/D816E Imatinib-Resistant Cells and GIST-T1/T670I Imatinib Resistant Cells

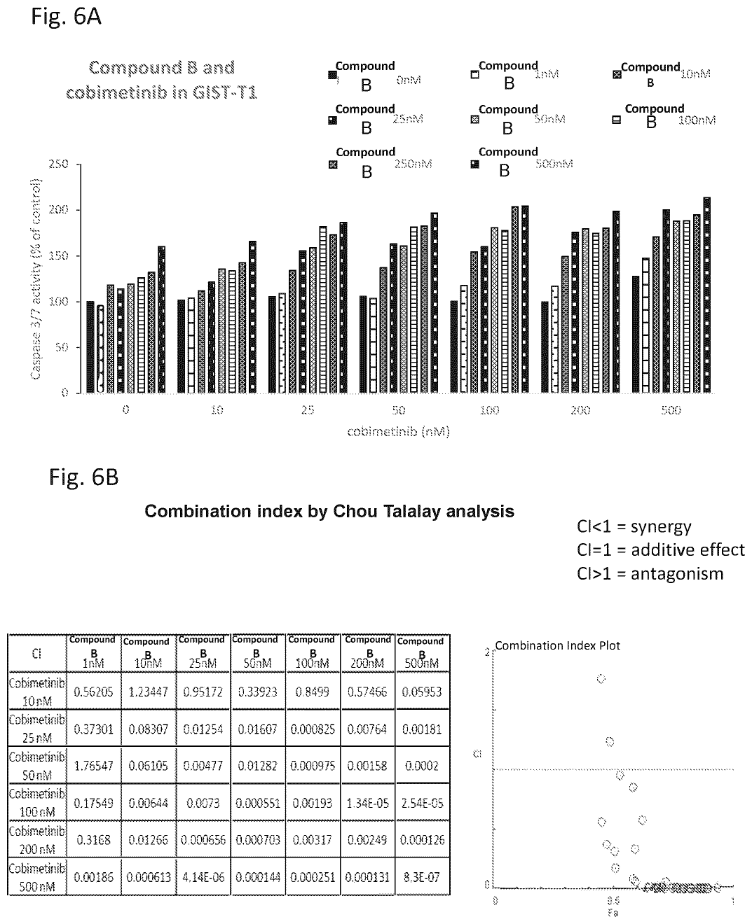

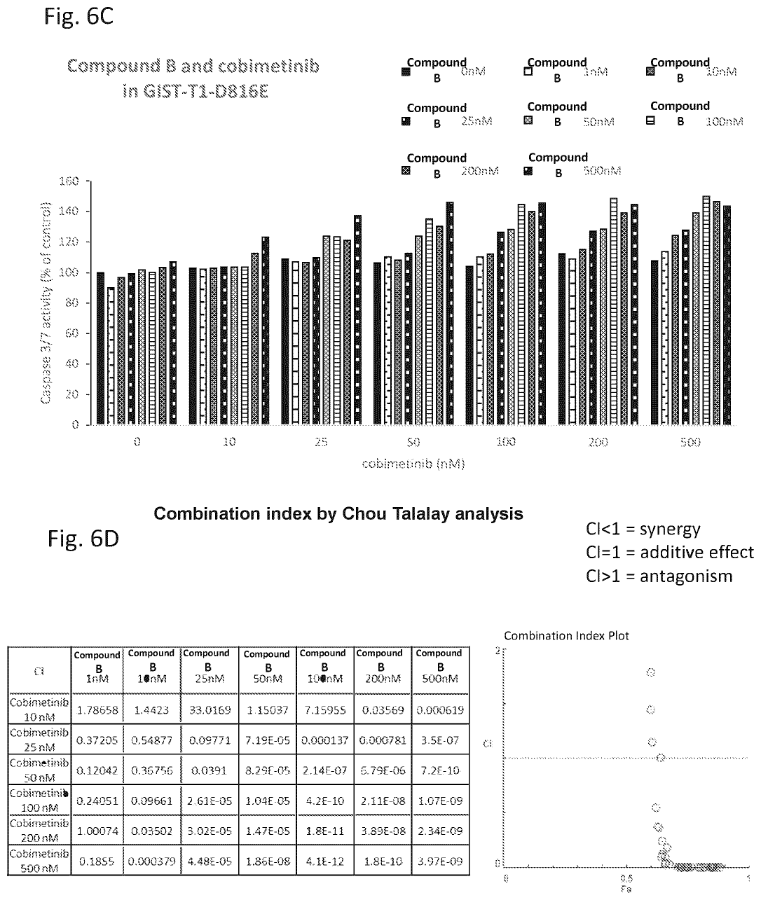

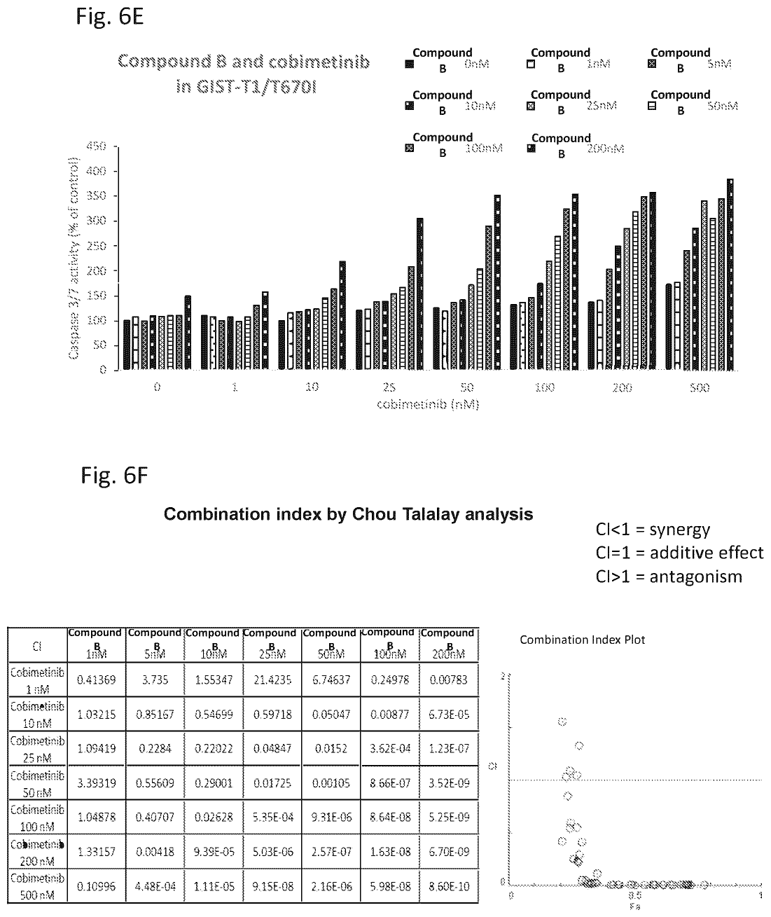

FIG. 6A is a graphical representations showing the relative percentage of caspase activity (compared to vehicle control set at 100%) determined for various treatments of GIST-T1 cells. FIG. 6B is a matrix synergy charts and combination index plot. Combination treatments for 24 hours (FIGS. 6A, 6B) with Compound B and cobimetinib showed strong synergy for inducing apoptosis in GIST-T1 cells.

FIG. 6C is a graphical representation showing caspase activity from various treatments of GIST-T1/D816E imatinib resistant cells. FIG. 6D is a matrix synergy chart and combination index plot. Combination treatments for 24 hours with Compound B and cobimetinib showed strong synergy for inducing apoptosis of GIST-T1/D816E imatinib resistant cells.

FIG. 6E is a graphical representation showing caspase activity from various treatments of GIST-T1/T670I imatinib resistant cells. FIG. 6F is a matrix synergy chart and combination index plot. Combination treatments for 24 hours with Compound B and cobimetinib showed strong synergy for inducing apoptosis of GIST-T1/T670I imatinib resistant cells.

### Example 7

### Combination Treatment of Compound A with Ulixertinib (BVD-523) Induces Apoptosis in GIST-T1, and GIST-T1/T670I Imatinib Resistant Cells

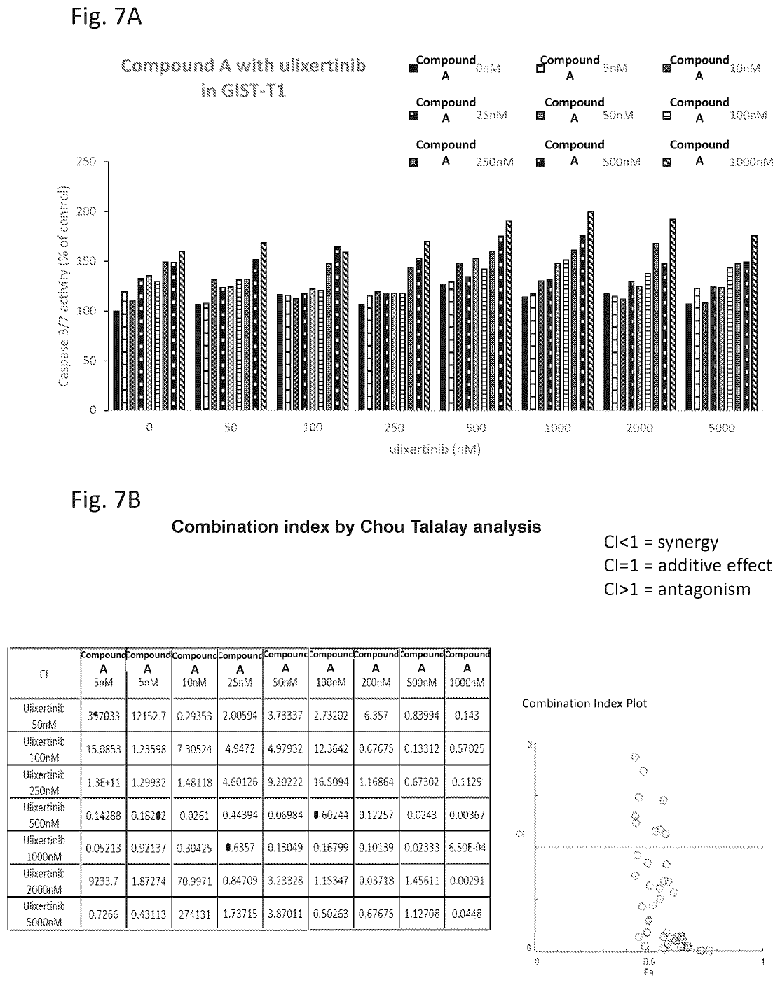

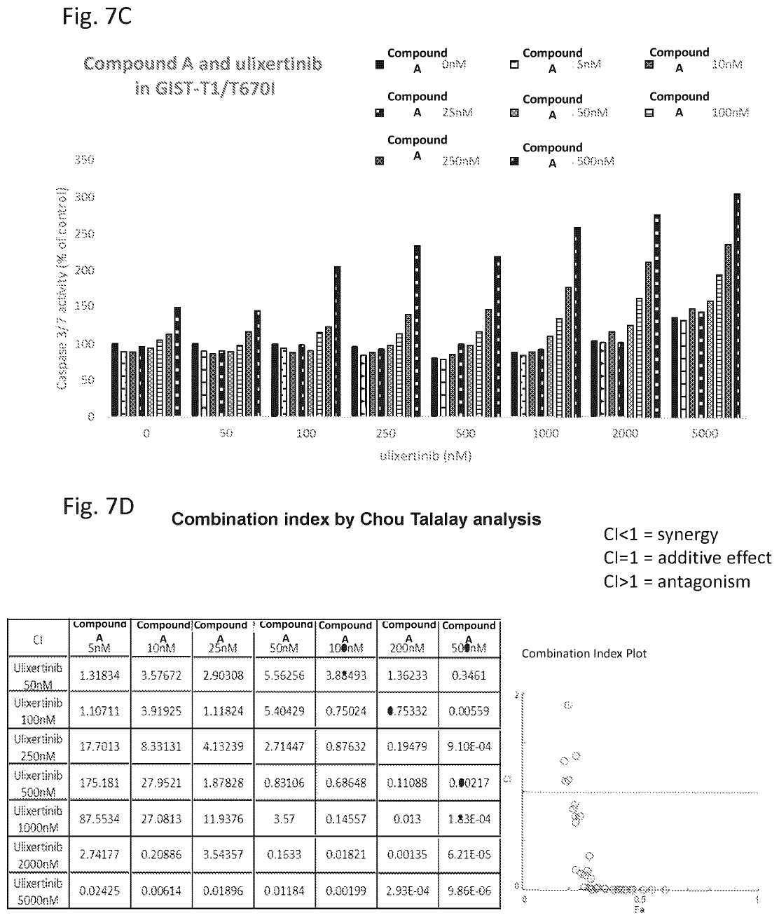

FIG. 7A is graphical representations showing the relative percentage of caspase activity (compared to vehicle control set at 100%) determined for various treatments of GIST-T1 cells. FIG. 7B shows matrix synergy charts and combination index plots based on the combination index (CI) method as described in example 1. Combination treatments for 24 hours (FIGS. 7A, 7B) with Compound A and ulixertinib showed strong synergy for inducing apoptosis in GIST-T1 cells at higher concentrations.

FIG. 7C is a graphical representation showing caspase activity from various treatments of GIST-T1/T670I imatinib resistant cells. FIG. 7D is a matrix synergy chart and combination index plot based on the combination index (CI) as described in example 1. Combination treatments for 24 hours with Compound A and ulixertinib showed strong synergy for inducing apoptosis of GIST-T1/T670I imatinib resistant cells.

### Example 8

### Combination Treatment of Compound B with Ulixertinib (Bvd-523) Induces Apoptosis in GIST-T1, and GIST-T1/T670I Imatinib Resistant Cells

Synergy charts and combination index plots for caspase activity can be used to show for synergy for Compound B and ulixertinib combination in inducing apoptosis in GIST-T1, GIST-T1/D816E imatinib resistant cells and GIST-T1/T670I Imatinib resistant cells.

### Example 9

### Combination Treatment of Compound A with SCH772984 Induces Apoptosis in GIST-T1, GIST-T1/D816E Imatinib-Resistant Cells and GIST-T1/T670I Imatinib Resistant Cells

Synergy charts and combination index plots for caspase activity can be used to show for synergy for Compound A and SCH772984 combination in inducing apoptosis in GIST-T1, GIST-T1/D816E imatinib resistant cells and GIST-T1/T670I imatinib resistant cells.

### Example 10

### Combination Treatment of Compound B with SCH772984 Induces Apoptosis in GIST-T1, GIST-T1/D816E Imatinib-Resistant Cells and GIST-T1/T670I Imatinib Resistant Cells

Synergy charts and combination index plots for caspase activity can be used to show for synergy for Compound B and SCH772984 combination in inducing apoptosis in GIST-T1, GIST-T1/D816E imatinib resistant cells and GIST-T1/T670I imatinib resistant cells.

### Example 11

### Combination Treatment of Compound A with LY3009120 Induces Apoptosis in GIST-T1, GIST-T1/D816E Imatinib-Resistant Cells and GIST-T1/T670I Imatinib Resistant Cells

Synergy charts and combination index plots for caspase activity can be used to show for synergy for Compound A and LY3009120 combination in inducing apoptosis in GIST-T1, GIST-T1/D816E imatinib resistant cells and GIST-T1/T670I imatinib resistant cells.

### Example 12

### Combination Treatment of Compound B with LY3009120 Induces Apoptosis in GIST-T1, GIST-T1/D816E Imatinib-Resistant Cells and GIST-T1/T670I Imatinib Resistant Cells

Synergy charts and combination index plots for caspase activity can be used to show for synergy for Compound B and LY3009120 combination in inducing apoptosis in GIST-T1, GIST-T1/D816E imatinib resistant cells and GIST-T1/T670I Imatinib resistant cells.

### Example 13

### Combination Treatment of Compound A with Dabrafenib Induces Apoptosis in GIST-T1, GIST-T1/D816E Imatinib-Resistant Cells and GIST-T1/T670I Imatinib Resistant Cells

Synergy charts and combination index plots for caspase activity can be used to show for synergy for Compound A and dabrafenib combination in inducing apoptosis in GIST-T1, GIST-T1/D816E imatinib resistant cells and GIST-T1/T670I Imatinib resistant cells.

### Example 14

### Combination Treatment of Compound B with Dabrafenib Induces Apoptosis in GIST-T1, GIST-T1/D816E Imatinib-Resistant Cells and GIST-T1/T670I Imatinib Resistant Cells

Synergy charts and combination index plots for caspase activity can be used to show for synergy for Compound B and dabrafenib combination in inducing apoptosis in GIST-T1, GIST-T1/D816E imatinib resistant cells and GIST-T1/T670I Imatinib resistant cells.

### Example 15

### Combination Treatment Prevents Colony Outgrowth in GIST-T1, GIST-T1/D816E and GIST-T1/T670I) Imatinib Resistant Cells

Studies were performed which demonstrate that combination treatment with Compound A and trametinib prevents colony outgrowth in GIST-T1 (57 bp exon 11 deletion) imatinib sensitive cells, GIST-T1/D816E imatinib resistant cells and GIST-T1/T670I imatinib resistant cells. Assays were conducted in 6 well plates with 100 cells seeded per well. Cells were treated with vehicle control, Compound A, trametinib, imatinib (IM), or combinations thereof at varying concentrations, and the cells were cultured for 2 weeks. Post-treatment, the drug was washed out, and the cells were cultured in normal media for 1-3 weeks. The outgrown cell colonies were stained with crystal violet and counted.

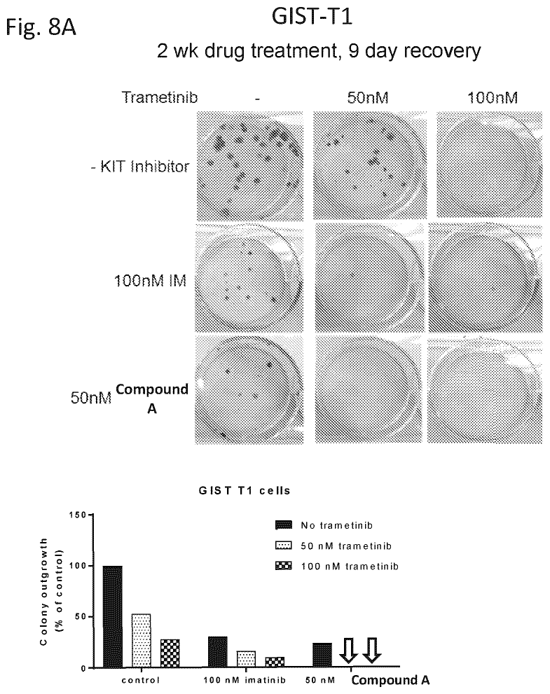

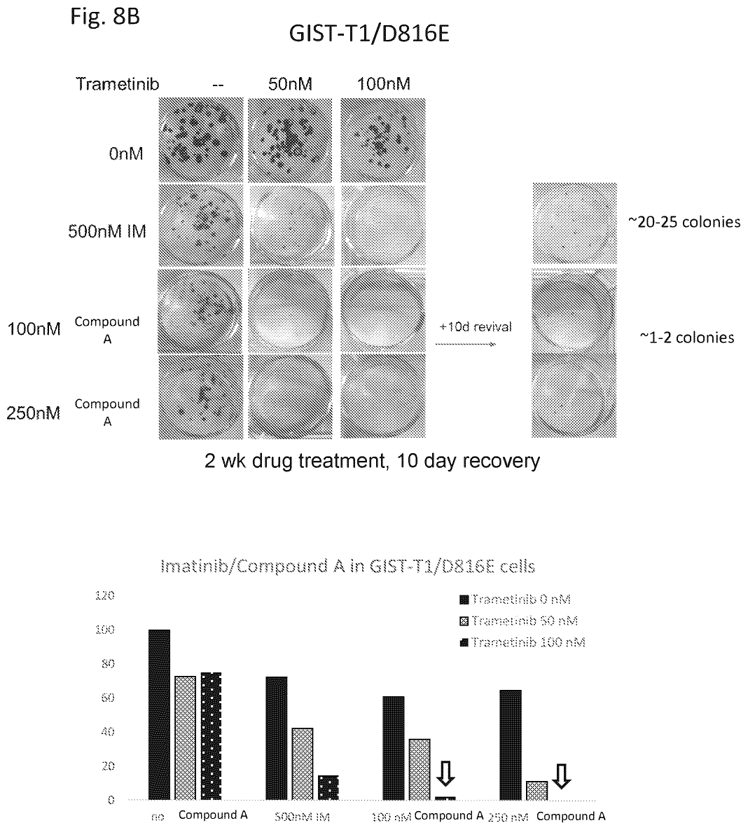

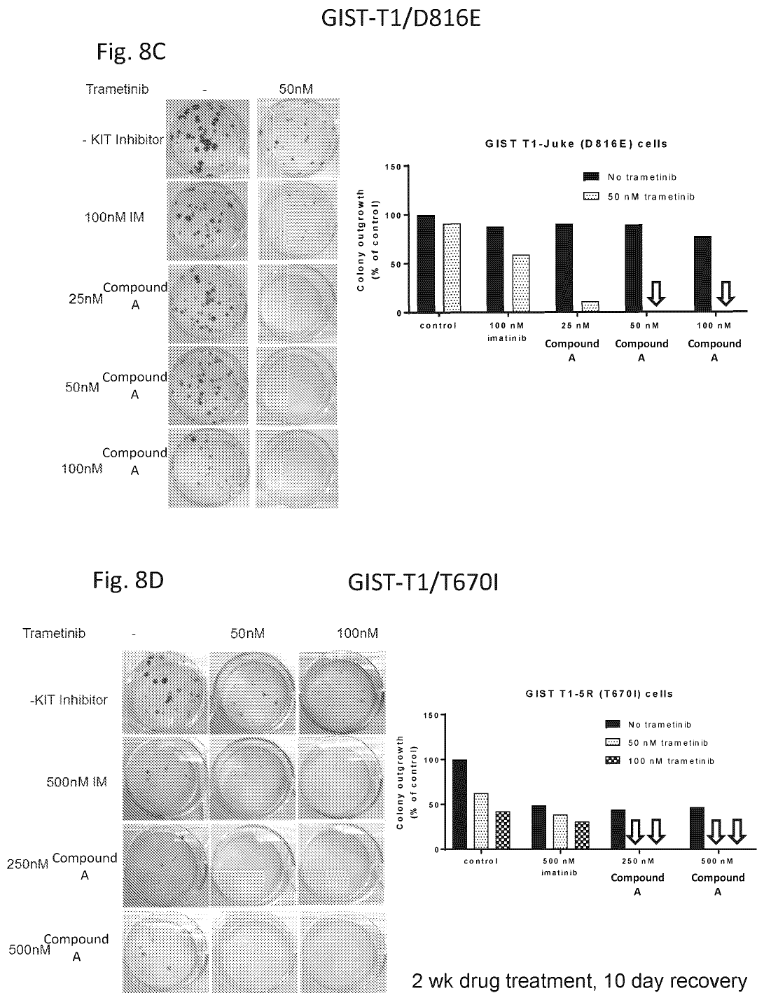

FIG. 8A shows images of representative culture plates and a graphical representation of the number of GIST-T1 colonies counted from various treatments. GIST T1 cells are sensitive to imatinib and Compound A as single agents. It is noted that each of imatinib and Compound A as single agents demonstrate approximately a similar reduction of GIST T1 colony outgrowth to 23-30% of vehicle control. Combination treatment for 2 weeks with 50 nM Compound A and either 50 nM or 100 nM trametinib unexpectedly led to complete cell stasis or eradication of GIST T1 colony outgrowth to the limit of detection as visualized with 5X objective microscopy, with no detectable colonies after removal of combination therapy for 9 days (note arrows in FIG. 8A). In contrast, combination treatment for 2 weeks with 100 nM imatinib and either 50 nM or 100 nM trametinib did not lead to complete tumor cell stasis or eradication after removal of combination therapy for 9 days.

FIG. 8B shows images of representative culture plates and graphical representation of the number of GIST-T1/D816E colonies counted from various treatments. It is noted that each of imatinib (500 nM) and Compound A (100 nM or 250 nM) as single agents demonstrate approximately a similar lack of cytocidal efficacy of GIST T1/D816E with colony outgrowth of approximately 61-72% of vehicle control (FIG. 8B). Combination treatment for 2 weeks with Compound A (100 nM or 250 nM) and trametinib (100 nM) led to almost complete cell stasis with Compound A (100 nM) and complete cell stasis or eradication of colony outgrowth in GIST-T1/D816E cells with combination of trametinib (100 nM) and Compound A (250 nM) to the limit of detection as visualized with 5X objective microscopy, following ten days of recovery (see arrows, FIG. 8B), whereas combination treatment for 2 weeks with imatinib (500 nM) and trametinib (50 nM or 100 nM) did not lead to complete cell stasis or tumor cell eradication (see graph in FIG. 8B). This was prominent when cells were cultured for an extra 10 days without drug and ˜20-25 colonies outgrew. FIG. 8C shows images of representative culture plates when Compound A concentration was further lowered to 25 nM, 50 nM, or 100 nM and evaluated in combination with 50 nM trametinib. Complete tumor cell stasis or eradication to the limit of detection as visualized with 5X objective microscopy of tumor colony outgrowth was achieved with 100 nM Compound A in combination with trametinib following ten days of recovery (see arrow, FIG. 8C), nearly complete tumor cell stasis or near eradication (1% of vehicle control) was achieved with 50 nM Compound A in combination with trametinib (see arrow, FIG. 8C), and significant tumor cell stasis or killing was achieved with 25 nM Compound A (11% of vehicle control). In contrast, combination of 100 nM imatinib with 50 nM trametinib did not eradicate tumor colony outgrowth, achieving a modest tumor cell stasis or killing following ten days of recovery (60% of vehicle control).

FIG. 8D shows images of representative culture plates and graphical representation of the number of GIST-T1/T670I colonies counted from various treatments. It is noted that each of imatinib (500 nM) and Compound A (250 nM or 500 nM) as single agents demonstrate approximately a similar reduction of GIST T1/T670I colony outgrowth to approximately 44-49% of vehicle control. Treatment for 2 weeks with either 250 nM or 500nM Compound A in combination with either 50 nM or 100 nM trametinib led to complete cell stasis or eradication of GIST T1/T670I colony outgrowth to the limit of detection as visualized with 5X objective microscopy, with no detectable colonies after removal of combination therapy for 10 days (note arrows in FIG. 8D). In contrast, treatment for 2 weeks with 500 nM imatinib in combination with either 50 nM or 100 nM trametinib did not lead to complete tumor cell stasis or tumor cell eradication after removal of combination therapy for 9 days.

### Example 16

### Combination Treatment of Compound B with Trametinib Prevents Colony Outgrowth in GIST-T1, GIST-T1/D816E and GIST-T1/T670I Imatinib Resistant Cells

Studies explained in example 8 were also performed in combination treatment with Compound B and trametinib in 3 GIST cell lines.

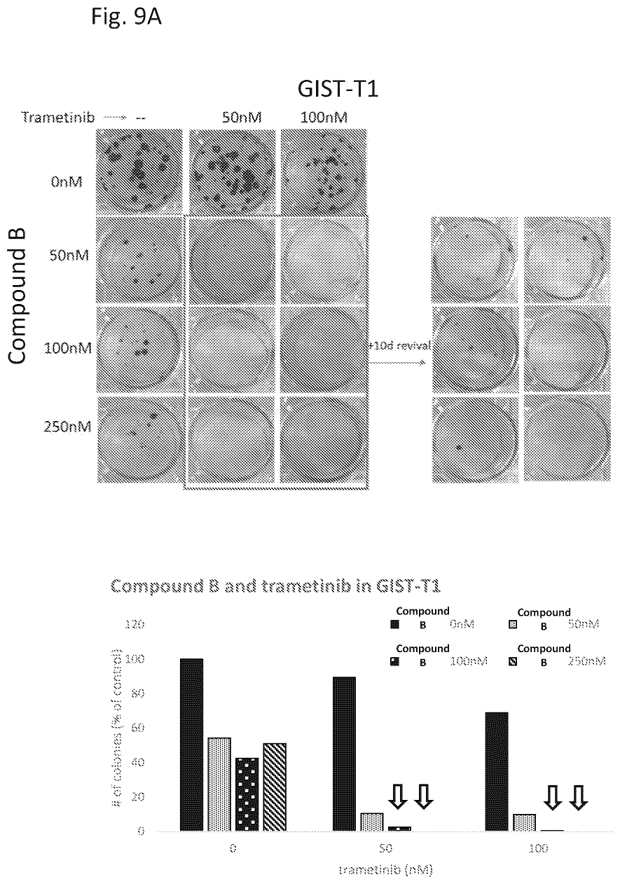

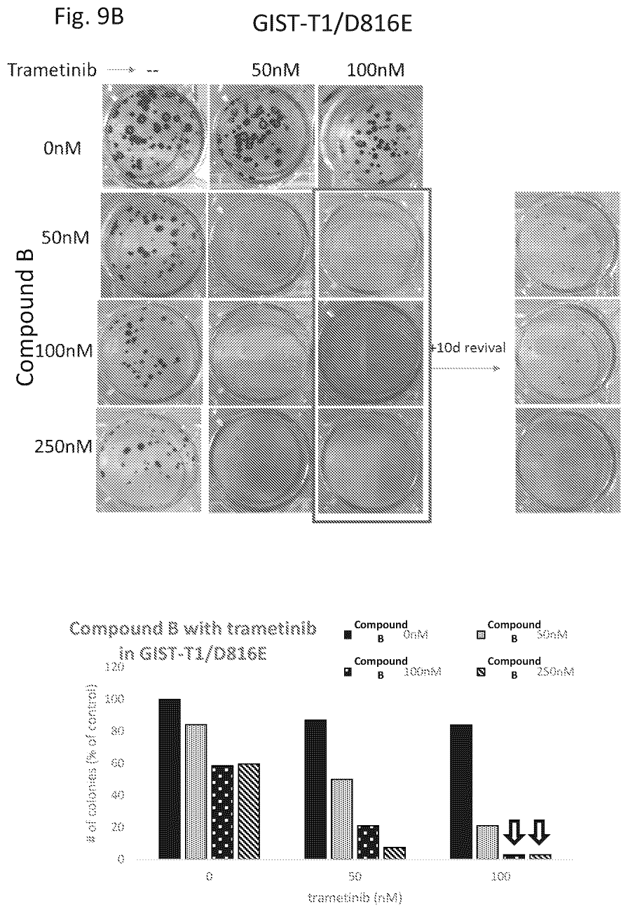

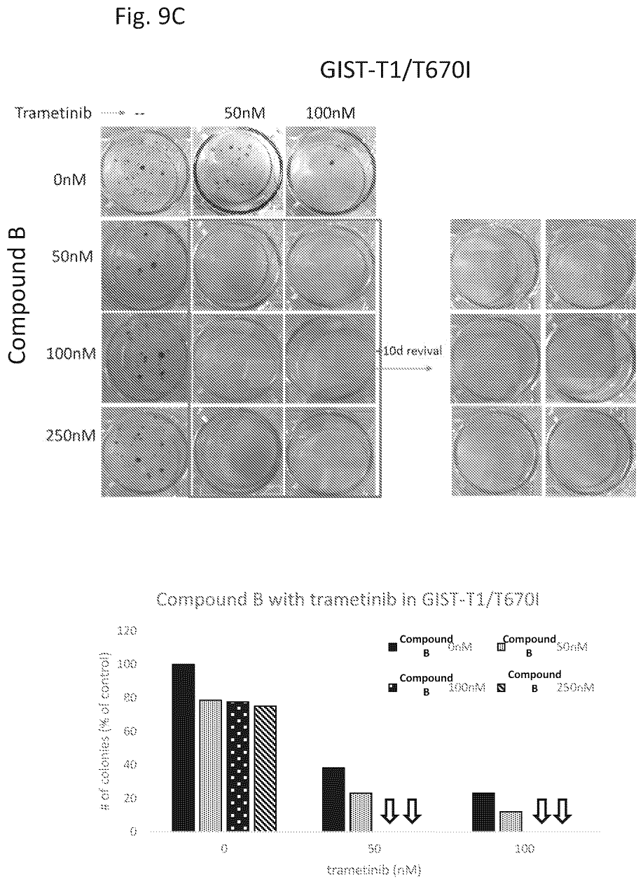

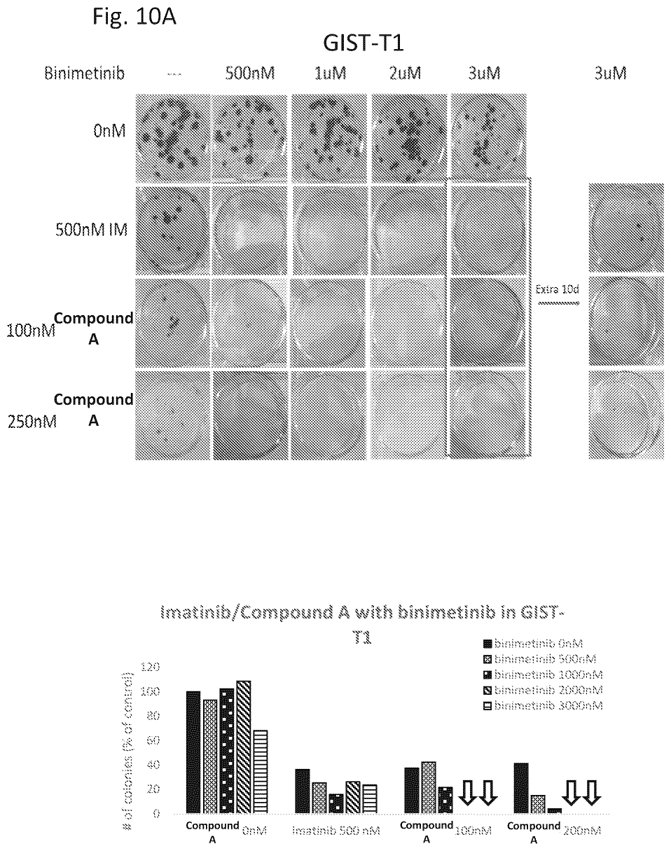

FIG. 9A shows images of representative culture plates and a graphical representation of the number of GIST-T1 colonies counted from various treatments. GIST T1 cells were sensitive to Compound B as single agents and showed a 42-54% reduction of GIST T1 colony outgrowth compared to vehicle control. Combination treatment for 2 weeks with 50 or 100 nM of Compound B and either 50 nM or 100 nM trametinib led to significant cell stasis with little colony outgrowh, while combination treatment with 250 nM of Compound A with either 50 nM or 100 nM trametninib led to complete cell stasis or eradication of GIST T1 colony outgrowth to the limit of detection as visualized with 5X objective microscopy, with no detectable colonies after removal of combination therapy for 10 days (note arrows in FIG. 9A). Outgrowth of colonies was prevented even after extended long term recovery for a total of 20 days.

FIG. 9B shows images of representative culture plates and graphical representation of the number of GIST-T1/D816E colonies counted from various treatments. It is noted that Compound B (50 nM, 100 nM or 250 nM) as a single agent demonstrate cytocidal efficacy of GIST T1/D816E with colony outgrowth of approximately 59-84% of vehicle control (FIG. 9B). Combination treatment for 2 weeks with Compound B (250 nM) and trametinib (50 nM) or with Compound B (100 nM or 250 nM) and trametinib (100 nM) led to >90% cell stasis or eradication of colony outgrowth in GIST-T1/D816E cells as visualized with 5X objective microscopy, following ten days of recovery (see arrows, FIG. 9B), Treatment with Compound B (250 nM) maintained cell stasis or cell killing in combination with trametinib (100 nM) even after extended long term of 20 days.

FIG. 9C shows images of representative culture plates and graphical representation of the number of GIST-T1/T670I colonies counted from various treatments. It is noted that Compound B (50 nM, 100 nM, 250 nM) as single agents demonstrate GIST T1/T670I colony outgrowth to approximately 75-78% of vehicle control. Treatment for 2 weeks with either 100 nM or 250 nM Compound B in combination with either 50 nM or 100 nM trametinib led to complete cell stasis or eradication of GIST T1/T670I colony outgrowth to the limit of detection as visualized with 5X objective microscopy, with no detectable colonies after removal of combination therapy for 10 days (note arrows in FIG. 9C). The inhibition of outgrowth was maintained even after extended term of 20 days without drug.

### Example 17

### Combination Treatment of Compound A with Binimetinib Prevents Colony Outgrowth in GIST-T1, GIST-T1/D816E and GIST-T1/T670I Imatinib Resistant Cells

Studies were performed which demonstrate that combination treatment with Compound A and binimetinib prevents colony outgrowth in 3 GIST cell lines as explained in example 15.

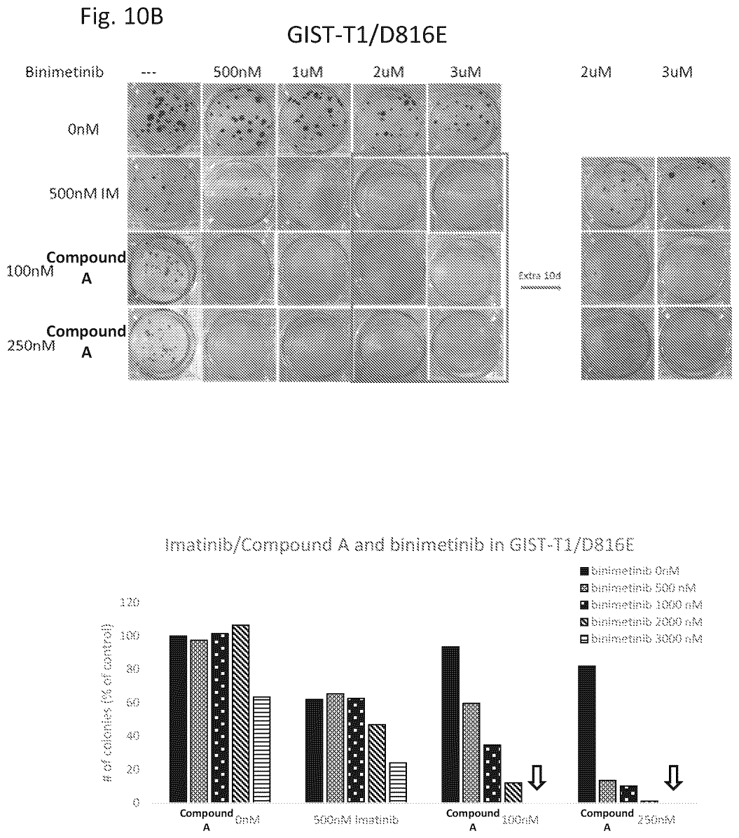

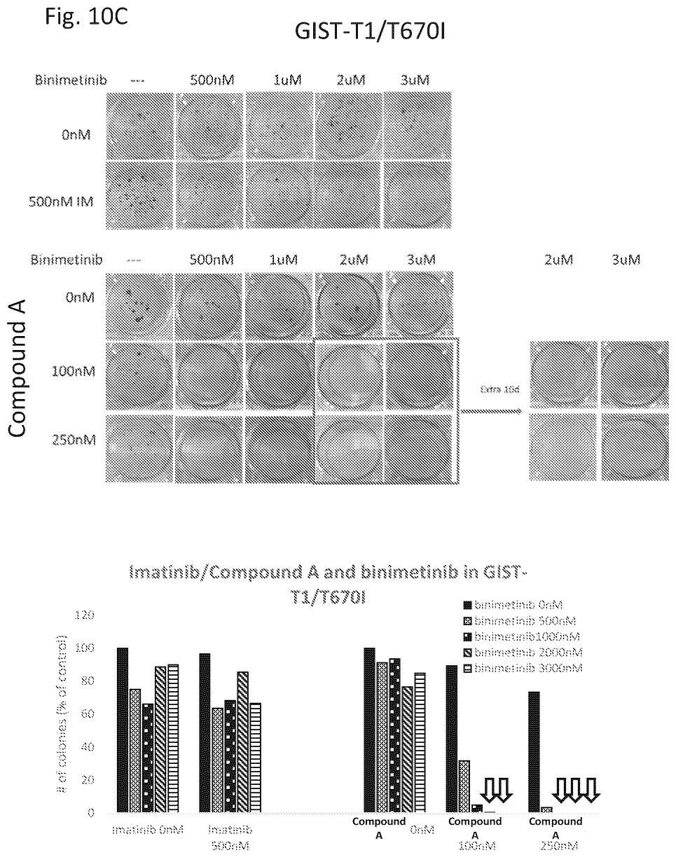

FIG. 10A shows images of representative culture plates and a graphical representation of the number of GIST-T1 colonies counted from various treatments. It is noted that each of imatinib and Compound A as single agents demonstrate approximately a similar reduction of GIST T1 colony outgrowth to 36-41% of vehicle control. Combination treatment for 2 weeks with 100 nM or 250 nM of Compound A and either 500 nM, 1 uM, 2 uM or 3 uM binimetinib was evaluated. Combination of Compound A (100 nM or 250 nM) with binimetinib (2 uM or 3 uM) led to complete cell stasis or eradication of GIST T1 colony outgrowth to the limit of detection as visualized with 5X objective microscopy, with no detectable colonies after removal of combination therapy for 10 days (note arrows in FIG. 10A). In contrast, combination treatment for 2 weeks with even 500 nM imatinib and either either 500 nM, 1 uM, 2 uM or 3 uM binimetinib did not lead to complete tumor cell stasis or eradication after removal of combination therapy for 10 days. The effect was more pronounced after incubation of extended period of time without drug, where ˜10-15 colonies were visible with imatinib and no colony outgrowth was observed with Compound A.

FIG. 10B shows images of representative culture plates and graphical representation of the number of GIST-T1/D816E colonies counted from various treatments. It is noted that each of imatinib (500 nM) and Compound A (100 nM or 250 nM) as single agents demonstrate a lack of cytocidal efficacy of GIST T1/D816E with colony outgrowth of approximately 60-95% of vehicle control (FIG. 10B). Combination treatment for 2 weeks with Compound A (100 nM or 250 nM) and binimetinib (500 nM, 1 uM, 2 uM or 3 uM) was evaluated. Combination of Compound A (100 nM or 250 nM) with binimetinib (3 uM) led to complete cell stasis or eradication of colony outgrowth in GIST-T1/D816E cells to the limit of detection as visualized with 5X objective microscopy, following ten days of recovery (see arrows, FIG. 10B), whereas combination treatment for 2 weeks with imatinib (500 nM) and binimetinib (500 nM, 1 uM, 2 uM or 3 uM) did not lead to complete cell stasis or tumor cell eradication. The effect was more pronounced after incubation of extended period of time where imatinib treatment did not lead to complete inhibition whwereas Compound A showed maintained cell stasis or cell killing even after 20 days.

FIG. 10C shows images of representative culture plates and graphical representation of the number of GIST-T1/T6701) colonies counted from various treatments. It is noted that imatinib (500 nM) as a single agent does not show any reduction of colonies whereas Compound A (100 nM or 250 nM) as single agents demonstrate a dose dependent reduction in colony outgrowth to about 78-89% of vehicle control. Treatment for 2 weeks with 250 nM Compound A in combination with 1 uM, 2 uM or 3 uM binimetinib led to complete cell stasis or eradication of GIST T1/T670I colony outgrowth to the limit of detection as visualized with 5X objective microscopy, with no detectable colonies after removal of combination therapy for 10 days (note arrows in FIG. 10C). Treatment for 2 weeks with 100 nM Compound A in combination with 3 uM binimetinib led to complete cell stasis or eradication of GIST T1/T670I colony outgrowth to the limit of detection as visualized with 5X objective microscopy, with no detectable colonies after removal of combination therapy for 10 days (note arrows in FIG. 10C). The cell stasis was maintained even after extended period of 20 days after drug removal. In contrast, treatment for 2 weeks with 500 nM imatinib in combination with either 500 nM, 1 uM, 2 uM or 3 uM binimetinib did not lead to complete tumor cell stasis or tumor cell eradication after removal of combination therapy for 10 days.

### Example 18

### Combination Treatment of Compound B with Binimetinib Prevents Colony Outgrowth in GIST-T1, GIST-T1/D816E and GIST-T1/T670I Imatinib Resistant Cells

Studies were performed which demonstrate that combination treatment with Compound B and binimetinib prevents colony outgrowth in 3 GIST cell lines as explained in Example 15.

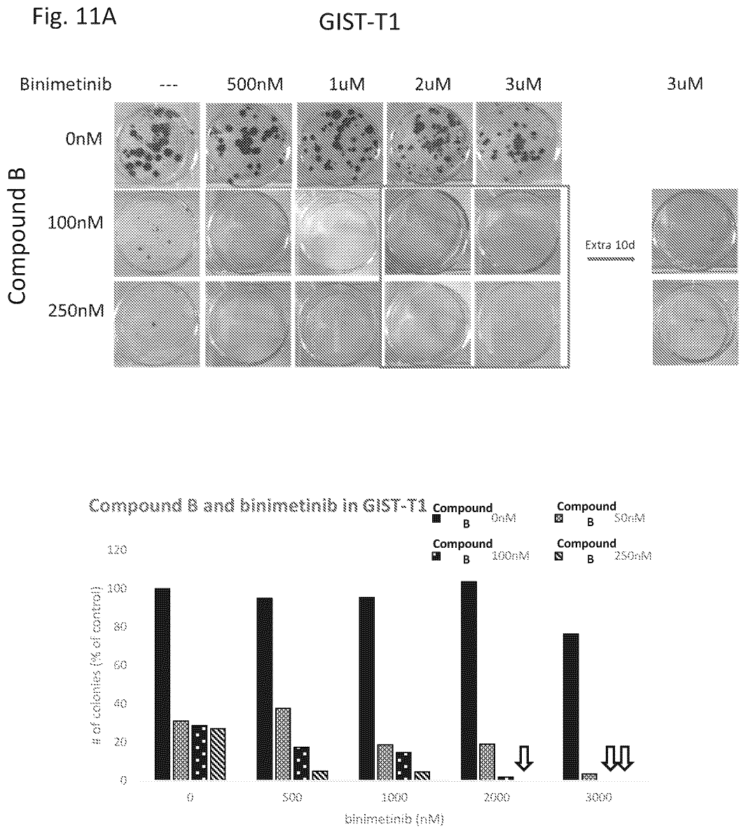

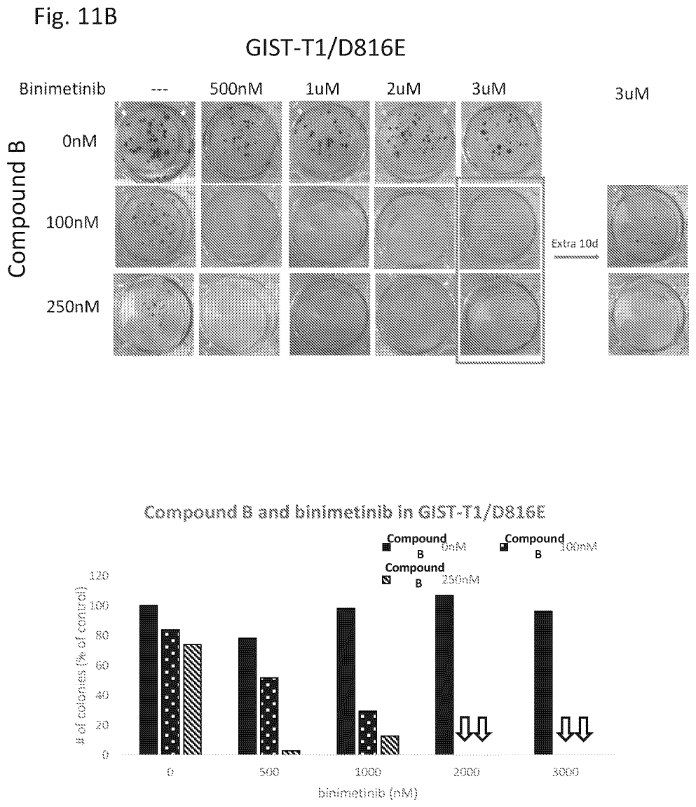

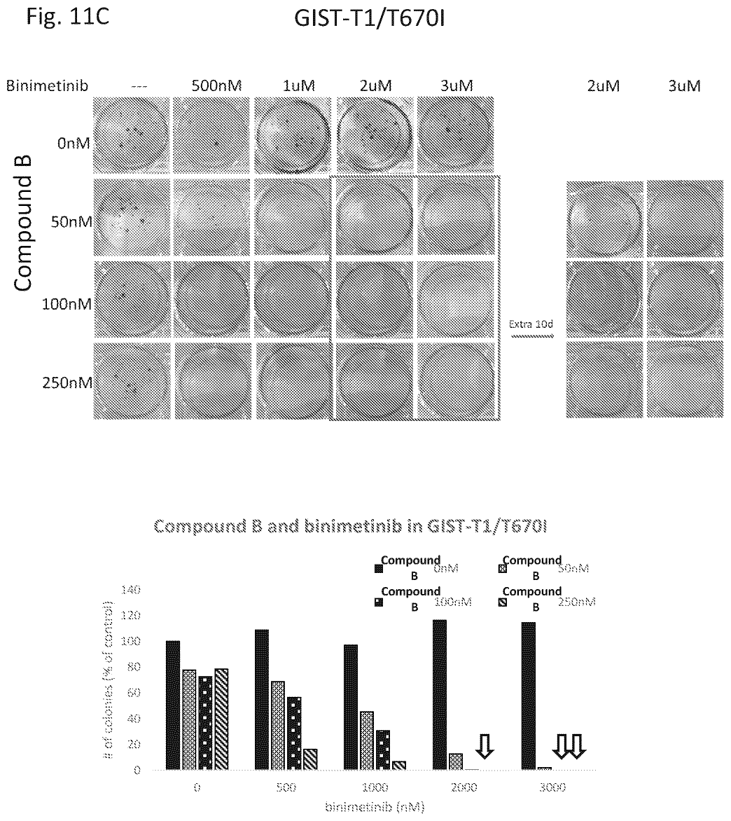

FIG. 11A shows images of representative culture plates and a graphical representation of the number of GIST-T1 colonies counted from various treatments. It is noted that each concentration of Compound B as a single agent demonstrates approximately a similar reduction of GIST T1 colony outgrowth to 27-31% of vehicle control. Combination treatment for 2 weeks with 250 nM of Compound B and 2 uM or 3 uM binimetinib led to complete cell stasis or eradication of GIST T1 colony outgrowth to the limit of detection as visualized with 5X objective microscopy, with no detectable colonies after removal of combination therapy for 10 days (note arrows in FIG. 11A) and maintained significant cell stasis or cell killing of GIST-T1 cells even after an extended long term recovery of 20 days (FIG. 11A upper right panel). Combination treatment for 2 weeks with 100 nM of Compound B and 3 uM binimetinib led to complete cell stasis or eradication of GIST T1 colony outgrowth to the limit of detection as visualized with 5X objective microscopy, with no detectable colonies after removal of combination therapy for 10 days (note arrows in FIG. 11A).

FIG. 11B shows images of representative culture plates and graphical representation of the number of GIST-T1/D816E colonies counted from various treatments. It is noted that Compound B (100 nM or 250 nM) as single agents demonstrate approximately a similar lack of cytocidal efficacy of GIST T1/D816E with colony outgrowth of approximately 74-83% of vehicle control (FIG. 11B). Combination treatment for 2 weeks with 100 nM or 250 nM of Compound B and either 2 uM or 3 uM binimetinib led to complete cell stasis or eradication of colony outgrowth in GIST-T1/D816E cells to the limit of detection as visualized with 5X objective microscopy, following ten days of recovery (see arrows, FIG. 11B) The cell stasis was maintained even after extended period of 20 days at highser concentration of Compound B.

FIG. 11C shows images of representative culture plates and graphical representation of the number of GIST-T1/T670I colonies counted from various treatments. It is noted that Compound B (100 nM or 250 nM) as single agent showed a reduction of GIST T1/T670I colony outgrowth to about 72-78% of vehicle control. Treatment for 2 weeks with either 100 nM or 250 nM of Compound B and 3 uM binimetinib unexpectedly led to complete cell stasis or eradication of GIST T1/T670I colony outgrowth to the limit of detection as visualized with 5X objective microscopy, with no detectable colonies after removal of combination therapy for 10 days (note arrows in FIG. 11C). Treatment for 2 weeks with 250 nM of Compound B and 2 uM binimetinib unexpectedly led to complete cell stasis or eradication of GIST T1/T670I colony outgrowth to the limit of detection as visualized with 5X objective microscopy, with no detectable colonies after removal of combination therapy for 10 days (note arrow in FIG. 11C). The cell stasis was maintained even after extended period of 20 days after drug removal.

### Example 19

### Combination Treatment of Compound A with Cobimetinib Prevents Colony Outgrowth in GIST-T1, GIST-T1/D816E and GIST-T1/T670I Imatinib Resistant Cells

Studies were performed which demonstrate that combination treatment with Compound A and cobimetinib prevents colony outgrowth in 3 GIST cell lines as explained in Example 15.

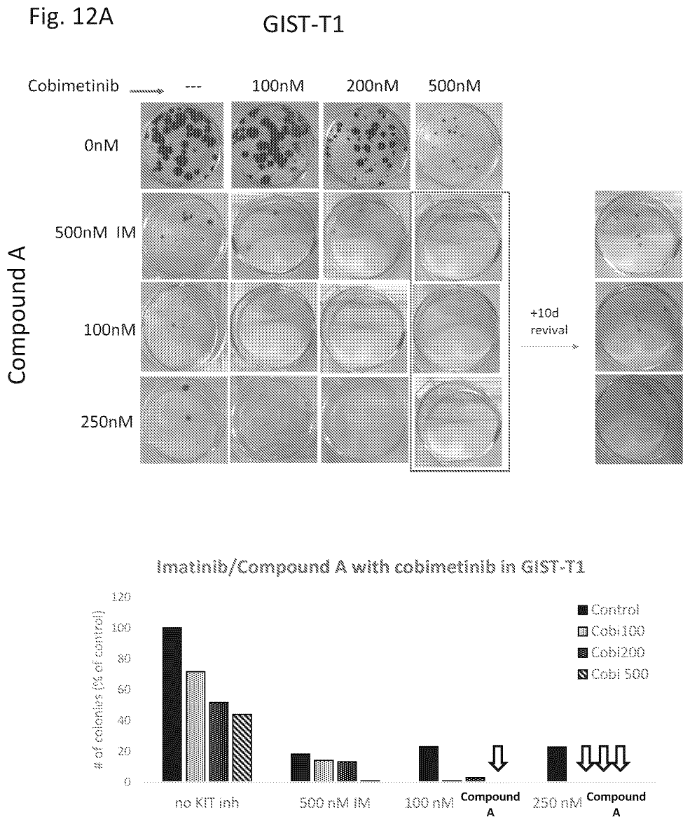

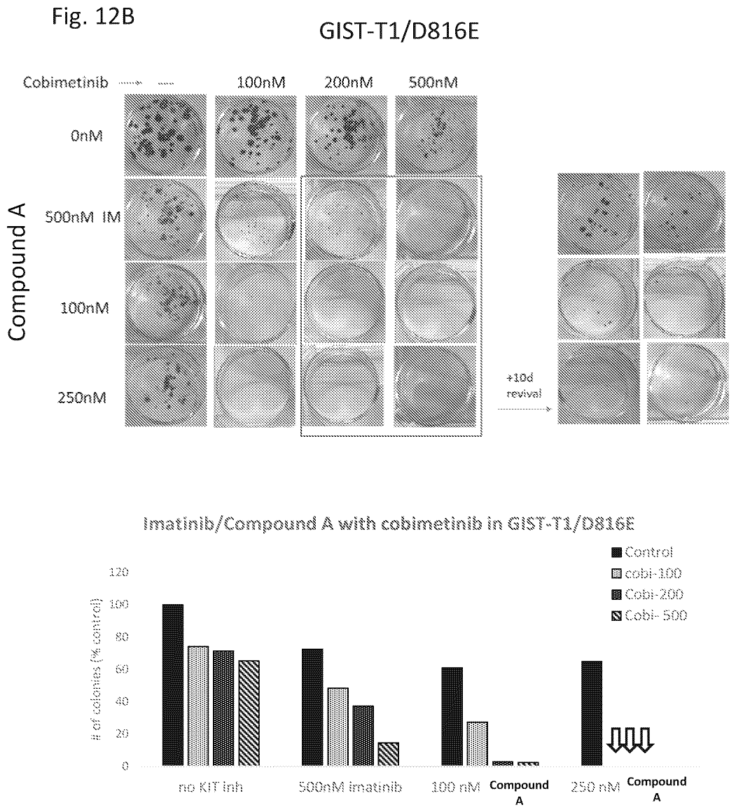

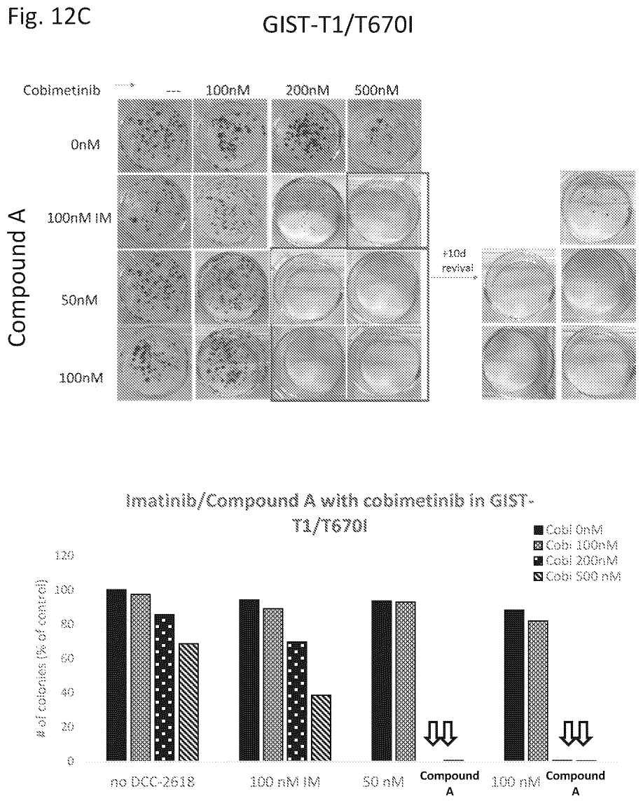

FIG. 12A shows images of representative culture plates and a graphical representation of the number of GIST-T1 colonies counted from various treatments. It is noted that each of imatinib and Compound A as single agents demonstrate approximately a similar reduction of GIST T1 colony outgrowth to 18-23% of vehicle control. Combination treatment for 2 weeks with 250 nM of Compound A and either 100 nM, 200 nM or 500 nM of cobimetinib led to complete cell stasis or eradication of GIST T1 colony outgrowth to the limit of detection as visualized with 5X objective microscopy, with no detectable colonies after removal of combination therapy for 10 days (note arrows in FIG. 12A). Combination treatment for 2 weeks with 100 nM of Compound A and 500 nM of cobimetinib led to complete cell stasis or eradication of GIST T1 colony outgrowth to the limit of detection as visualized with 5X objective microscopy, with no detectable colonies after removal of combination therapy for 10 days (note arrow in FIG. 12A). In contrast, combination treatment for 2 weeks with 500 nM imatinib and either 100 nM, 200 nM or 500 nM cobimetinib did not lead to complete tumor cell stasis or eradication after removal of combination therapy for 10 days.

The effect was more visible after incubation of extended period of time where ˜10-15 colonies grew out with 500 nM imatinib and no colony out growth was observed with 500 nM of Compound A and 500 nM of cobimetinib.

FIG. 12B shows images of representative culture plates and graphical representation of the number of GIST-T1/D816E colonies counted from various treatments. It is noted that each of imatinib (500 nM) and Compound A (100 nM or 250 nM) as single agents demonstrate approximately a similar lack of cytocidal efficacy of GIST T1/D816E with colony outgrowth of approximately 65-74% of vehicle control (FIG. 12B). Combination treatment for 2 weeks with Compound A (250 nM) and cobimetinib (100 nM, 200 nM or 500 nM) led to complete cell stasis or eradication of colony outgrowth in GIST-T1/D816E cells to the limit of detection as visualized with 5X objective microscopy, following ten days of recovery (see arrows, FIG. 12B), whereas combination treatment for 2 weeks with imatinib (500 nM) and cobimetinib (100 nM, 200 nM or 500 nM) did not lead to complete cell stasis or tumor cell eradication. The effect was more visible after incubation of extended period of time where imatinib treatment did not lead to complete inhibition whereas Compound A showed maintained significant cell stasis or cell killing even after 20 days after drug removal.

FIG. 12C shows images of representative culture plates and graphical representation of the number of GIST-T1/T670I colonies counted from various treatments. Treatment for 2 weeks with either 50 nM or 100 nM Compound A in combination with cobimetinib (200 nM or 500 nM) unexpectedly led to >99% inhibition of GIST T1/T670I colony outgrowth as visualized with 5X objective microscopy (note arrows in FIG. 12C). The cell stasis was maintained even after extended period of 20 days after drug removal. In contrast, treatment for 2 weeks with 500 nM imatinib in combination with up to 500 nM of cobimetinib did not lead to robust cell stasis or cell eradication after removal of combination therapy for 10 days.

### Example 20

### Combination Treatment of Compound B with Cobimetinib Prevents Colony Outgrowth in GIST-T1, GIST-T1/D816E and GIST-T1/T670I Imatinib Resistant Cells

Studies were performed which demonstrate that combination treatment with Compound B and cobimetinib prevents colony outgrowth in 3 GIST cell lines as explained in example 15.

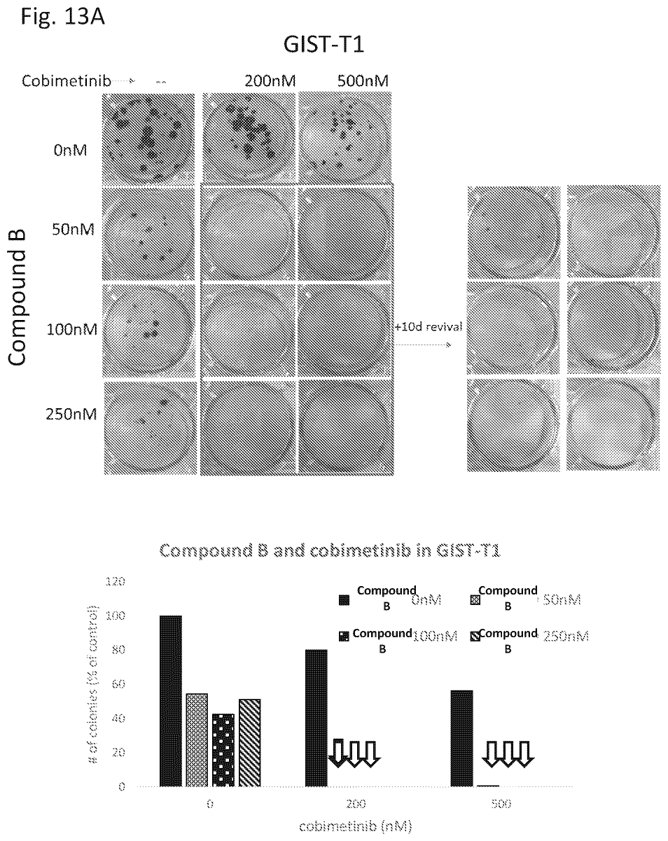

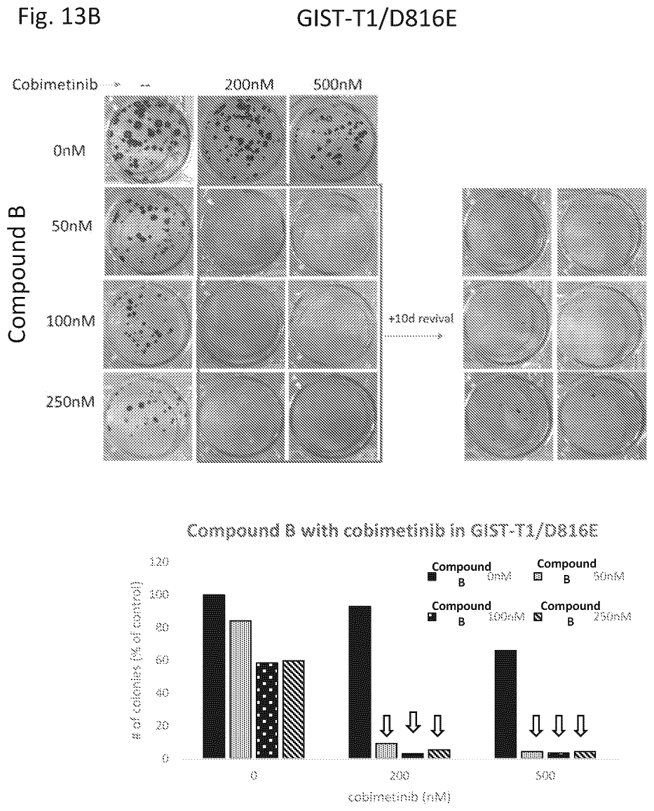

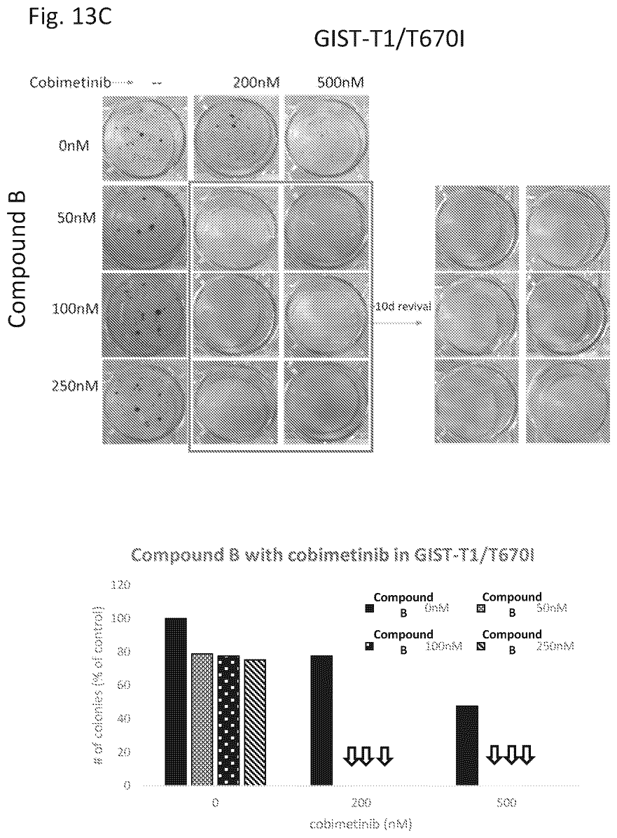

FIG. 13A shows images of representative culture plates and a graphical representation of the number of GIST-T1 colonies counted from various treatments. It is noted that Compound B as a single agent demonstrate approximately a similar reduction of GIST T1 colony outgrowth to 42-54% of vehicle control. Combination treatment for 2 weeks with 50 nM, 100 nM or 250 nM of Compound B and either 200 nM or 500 nM of cobimetinib led to complete or near complete cell stasis or eradication of GIST T1 colony outgrowth to the limit of detection as visualized with 5X objective microscopy, with no detectable colonies after removal of combination therapy for 10 days (note arrows in FIG. 13A). Combination treatment for 2 weeks with with 100 nM or 250 nM of Compound B and either 200 nM or 500 nM of cobimetinib maintained significant cell stasis or cell killing of GIST-T1 cells even after an extended long term recovery of 20 days.

FIG. 13B shows images of representative culture plates and graphical representation of the number of GIST-T1/D816E colonies counted from various treatments. It is noted that Compound B (100 nM or 250 nM) as a single agent demonstrates a lack of cytocidal efficacy of GIST T1/D816E with colony outgrowth of approximately 58-84% of vehicle control (FIG. 13B). Combination treatment for 2 weeks with 50 nM, 100 nM or 250 nM of Compound B and either 200 nM or 500 nM of cobimetinib led to >90 inhibition of colony outgrowth in GIST-T1/D816E cells as visualized with 5X objective microscopy, following ten days of recovery (see arrows, FIG. 13B). The cell stasis was significantly maintained even after extended period of 20 days at higher concentration of Compound B.

FIG. 13C shows images of representative culture plates and graphical representation of the number of GIST-T1/T670I colonies counted from various treatments. It is noted that Compound B (100 nM or 250 nM) as single showed a reduction of GIST T1/T670I colony outgrowth to about 75-78% of vehicle control. Treatment for 2 weeks with either 50 nM, 100 nM or 250 nM of Compound B and either 200 nM or 500 nM of cobimetinib led to complete cell stasis or eradication of GIST T1/T670I colony outgrowth to the limit of detection as visualized with 5X objective microscopy, with no detectable colonies after removal of combination therapy for 10 days (note arrows in FIG. 13C). The cell stasis was maintained even after extended period of 20 days after drug removal.

### Example 21

### Combination Treatment of Compound A with Ulixertinib Prevents Colony Outgrowth in GIST-T1, GIST-T1/D816E and GIST-T1/T670I Imatinib Resistant Cells

Studies were performed which demonstrate that combination treatment with Compound A and ulixertinib prevents colony outgrowth in 3 GIST cell lines as explained in example 15.

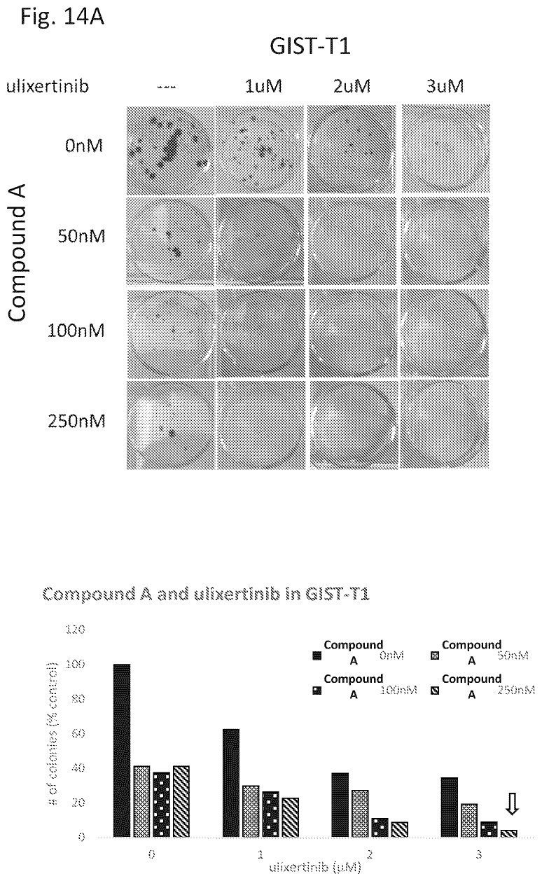

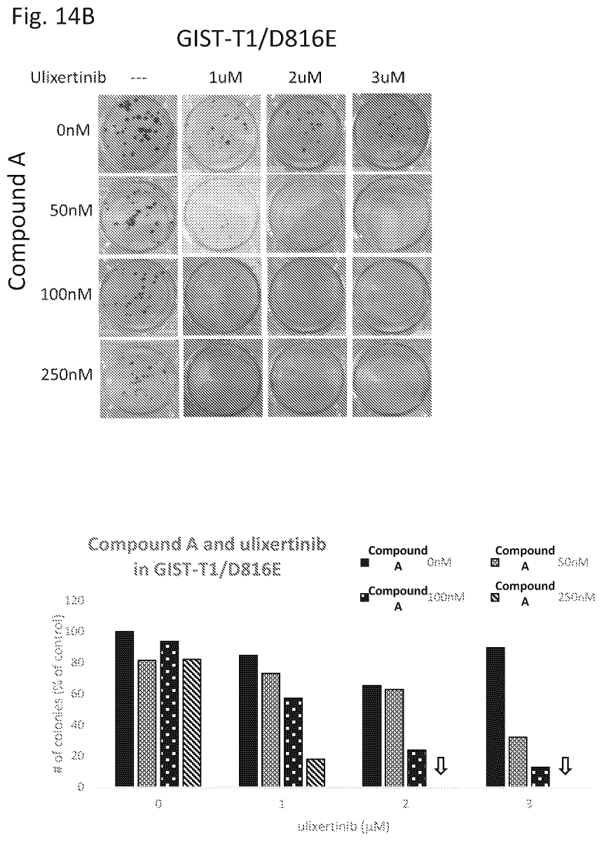

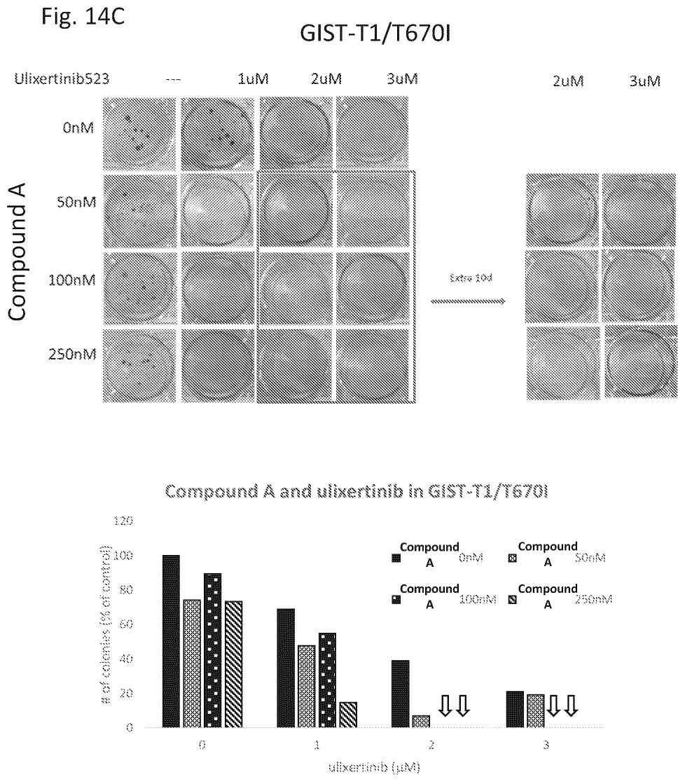

FIG. 14A shows images of representative culture plates and a graphical representation of the number of GIST-T1 colonies counted from various treatments. It is noted that Compound A as single agents demonstrate approximately a similar reduction of GIST T1 colony outgrowth to 37-41% of vehicle control. Combination treatment for 2 weeks with 50 nM, 100 nM or 250 nM of Compound A and either 1 uM, 2 uM or 3 uM of ulixertinib led to significant decrease in GIST T1 colony outgrowth as visualized with 5X objective microscopy (note arrows in FIG. 14A).

FIG. 14B shows images of representative culture plates and graphical representation of the number of GIST-T1/D816E colonies counted from various treatments. It is noted that Compound A (50 nM, 100 nM or 250 nM) as single agent demonstrate approximately a similar lack of cytocidal efficacy of GIST T1/D816E with colony outgrowth of approximately 81-93% of vehicle control (FIG. 14B). Combination treatment for 2 weeks with Compound A (250 nM) and ulixertinib (2 uM or 3 uM) led to complete cell stasis or eradication of colony outgrowth in GIST-T1/D816E cells to the limit of detection as visualized with 5X objective microscopy, following ten days of recovery (see arrows, FIG. 14B)

FIG. 14C shows images of representative culture plates and graphical representation of the number of GIST-T1/T670I colonies counted from various treatments. Treatment for 2 weeks with either 100 nM or 250 nM Compound A in combination with ulixertinib (2 uM or 3 uM) unexpectedly led to complete cell stasis or eradication of GIST T1/T670I colony outgrowth to the limit of detection as visualized with 5X objective microscopy, with no detectable colonies after removal of combination therapy for 10 days (note arrows in FIG. 14C). The cell stasis was maintained even after extended period of 20days after drug removal.

### Example 22

### Combination Treatment Of Compound B with Ulixertinib Prevents Colony Outgrowth in GIST-T1/D816E Imatinib Resistant Cells

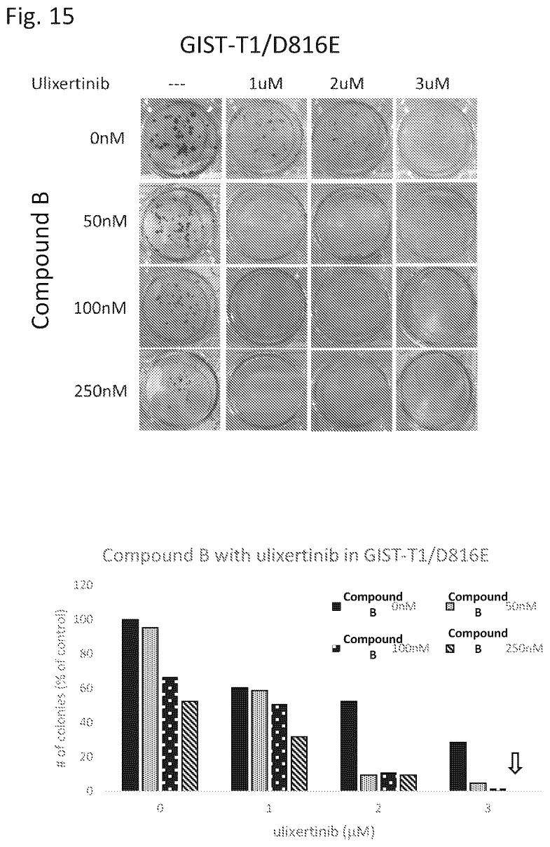

FIG. 15 shows images of representative culture plates and graphical representation of the number of GIST-T1/D816E colonies counted from various treatments. It is noted that Compound B (50 nM, 100 nM or 250 nM) as a single agent demonstrated approximately a similar lack of cytocidal efficacy of GIST T1/D816E with colony outgrowth of approximately 52-95% of vehicle control (FIG. 15). Combination treatment for 2 weeks with Compound B (250 nM) and ulixertinib (3 uM) led to complete cell stasis or eradication of GIST T1/D816E colony outgrowth to the limit of detection as visualized with 5X objective microscopy, with no detectable colonies after removal of combination therapy for 10 days (note arrow in FIG. 15).

### Example 23

### Combination Treatment of Compound A and ERK Inhibitor SCH772984Prevents Colony Outgrowth in GIST-T1/D816E Imatinib Resistant Cells

The protocol outlined in Example 15 can be used to show synergy for Compound A and SCH772984 combination in preventing outgrowth of colonies in GIST-T1, GIST-T1/T670I and GIST-T1/D816E imatinib resistant cells.

### Example 24

### Combination Treatment of Compound B and ERK Inhibitor SCH772984Prevents Colony Outgrowth in GIST-T1/D816E Imatinib Resistant Cells

The protocol outlined in Example 15 can be used to show synergy for Compound B and SCH772984 combination in preventing outgrowth of colonies in GIST-T1, GIST-T1/T670I and GIST-T1/D816E imatinib resistant cells.

### Example 25

### Combination Treatment of Compound a and RAF Inhibitor LY3009120 Prevents Colony Outgrowth in GIST-T1/D816E Imatinib Resistant Cells

The protocol outlined in Example 15 can be used to show synergy for Compound A and LY3009120 combination in preventing outgrowth of colonies in GIST-T1, GIST-T1/T670I and GIST-T1/D816E imatinib resistant cells.

### Example 26

### Combination Ttreatment of Compound B and RAF Inhibitor LY3009120 Prevents Colony Outgrowth in GIST-T1/D816E Imatinib Resistant Cells

The protocol outlined in Example 15 can be used to show synergy for Compound B and LY3009120 combination in preventing outgrowth of colonies in GIST-T1, GIST-T1/T670I and GIST-T1/D816E imatinib resistant cells.

### Example 27

### Combination Treatment of Compound A and RAF Inhibitor Inhibitor Dabrafenib Prevents Colony Outgrowth in GIST-T1/D816E Imatinib Resistant Cells

The protocol outlined in Example 15 can be used to show synergy for Compound A and dabrafenib combination in preventing outgrowth of colonies in GIST-T1, GIST-T1/T6701 and GIST-T1/D816E imatinib resistant cells.

### Example 28

### Combination Ttreatment of Compound B and RAF Inhibitor Inhibitor Dabrafenib Prevents Colony Outgrowth in GIST-T1/D816E Imatinib Resistant Cells

The protocol outlined in Example 15 can be used to show synergy for Compound B and dabrafanib combination in preventing outgrowth of colonies in GIST-T1, GIST-T1/T670I and GIST-T1/D816E imatinib resistant cells.

### Example 29

### Combination Treatment Induces Apoptosis in N-ras G12D Transfected GIST-T1 Cells

A study was performed which demonstrates that combination treatment of Compound A and trametinib induces apoptosis in empty vector control (EV) and mutant N-ras G12D transfected GIST-T1 cells. Assays were conducted in 96 well plates with 10,000 cells seeded/well for vector control or N-ras G12D transfected GIST-T1 cells. The cells were treated with vehicle control, Compound A, trametinib, or combinations thereof at varying concentrations, and the cells were allowed to grow for 48 hours. Apoptosis was assessed by measuring caspase 3/7 activity.

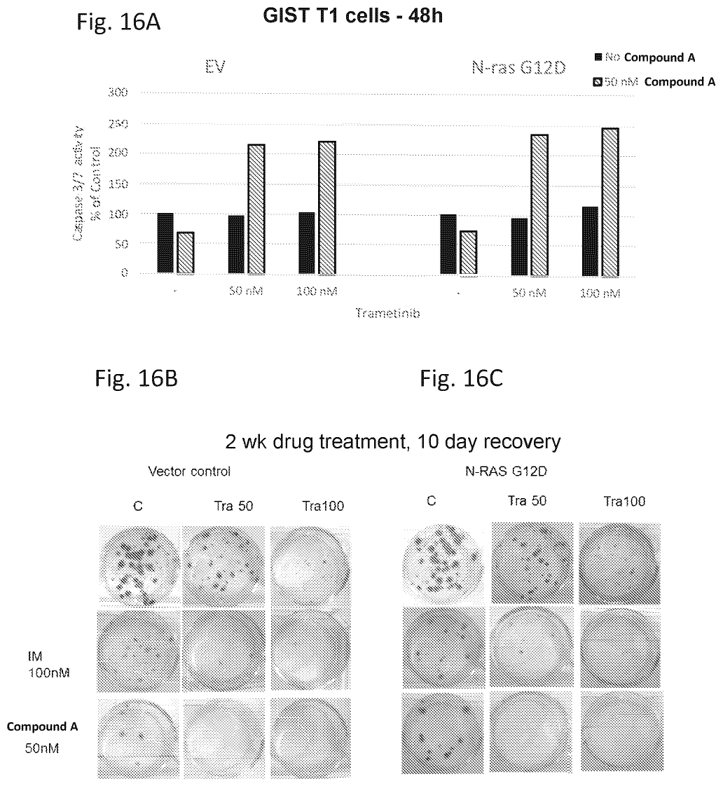

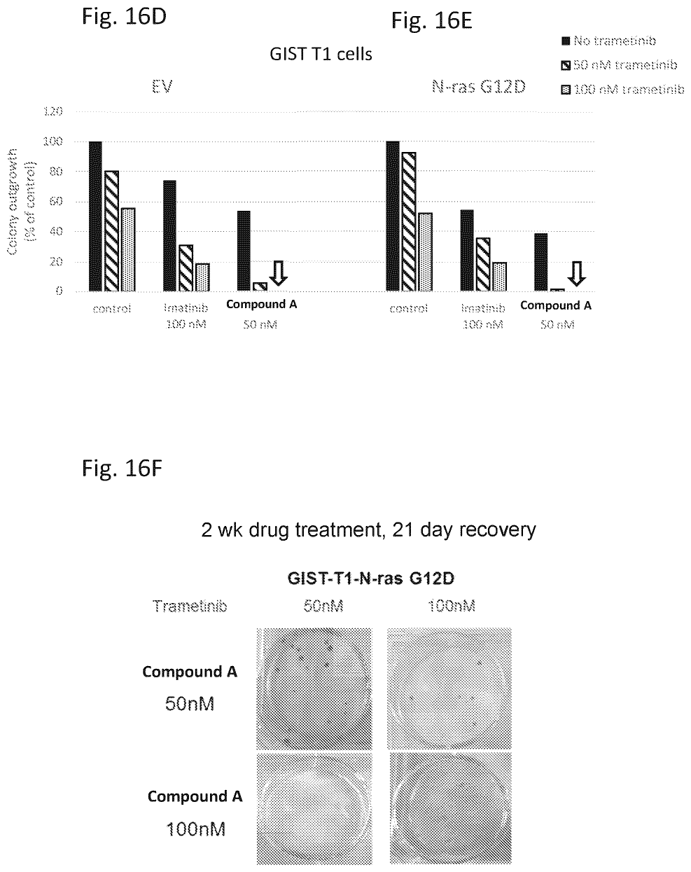

FIG. 16A provides graphical representations of caspase activity measure after the various treatments. Combination treatment for 48 hours with 50 nM Compound A and trametinib (50 nM or 100 nM) induced an increased apoptosis in mutant N-ras G12D transfected GIST-T1 cells compared to cells treated with either single agent Compound A or trametinib.

### Example 30

### Combination Treatment Prevents Colony Outgrowth in N-ras G12D Transfected GIST-T1 Cells

A study was performed which demonstrates that combination treatment of Compound A and trametinib prevents resistant colony outgrowth in empty vector control and mutant N-ras G12D transfected GIST-T1 cells. Assays were conducted in 6 well plates with 100 cells seeded per well. Cells were treated with vehicle control, 50 nM Compound A, 50 nM or 100 nM trametinb, or combinations thereof, and the cells were cultured for 2 weeks. In the same experiment, cells were treated with vehicle control, 100 nM imatinib, 50 nM or 100 nM trametinib, or combinations thereof. After 2 weeks, the drug was washed out, and the cells were cultured in normal media for 1-3 weeks. The colonies were stained with crystal violet and counted.

FIG. 16B and FIG. 16C show images of representative culture plates, and FIG. 16D and FIG. 16F show graphical representations of the number of vector control (FIG. 16B) and mutant N-ras G12D (FIG. 16C) transfected GIST-T1 colonies counted following various treatments. Quantitation of colony outgrowth in the vector control and N-ras G12D transfected GIST T-1 cells is shown in FIG. 16D and FIG. 16E, respectively. Combination treatment with 100 nM imatinib and 50 nM trametinib resulted in colony outgrowth (35% of vehicle control), and combination of 100 nM imatinib with 100 nM trametinib also resulted in colony outgrowth (19% of vehicle). In contrast, combinations of Compound A with trametinib unexpectedly resulted in superior cell stasis or cell killing compared to combination with imatinib. Combination treatment for 2 weeks with 50 nM Compound A and 50 nM trametinib led to almost complete cell stasis or cell killing (2% of vehicle control), and combination of 50 nM Compound A with 100 nM trametinib led to complete (0% of vehicle control colony outgrowth) cell stasis or cell killing to the limit of detection as visualized with 5X objective microscopy following ten days of drug washout and recovery (see arrow, FIG. 16E).

FIG. 16F shows images of representative culture plates of the number of mutant N-ras G12D transfected GIST-T1 colonies counted following an extended drug-free recovery period. Combination treatment for 2 weeks with 100 nM Compound A and 50 nM or 100 nM trametinib led to near complete blockade of colony outgrowth in N-ras G12D transfected GIST-T1 cells after an extended long term recovery period of 21 days.

### Example 32

### Combination Treatment Prevent Colony Outgrowth in Drug Resistant GIST Cells

A saturation mutagenesis study was performed in Ba/F3 cells transformed with oncogenic KIT V560D mutant. DNA nicking was induced by N-ethyl-N-nitrosourea (ENU) for 18 hours to generate additional mutations in the KIT gene or other genes in a random fashion. Assays were conducted in 6 well plates with 100 cells seeded per well. After washout of ENU, wells were incubated with 100 nM or 250 nM or 500 nM imatinib, 100 nM or 250 nM or 500 nM imatinib in combination with 10 nM trametinib, 25 nM or 100nM or 250 nM Compound A, or a combination of 25 nM, 100 nM, or 250 nM Compound A with 10 nM trametinib. Wells which exhibited resistance to drug treatments exhibited outgrowth of Ba/F3 cells. These cells were subjected to PCR and sequencing of the KIT gene to determine the presence of a resistant secondary mutation induced by the ENU treatment.

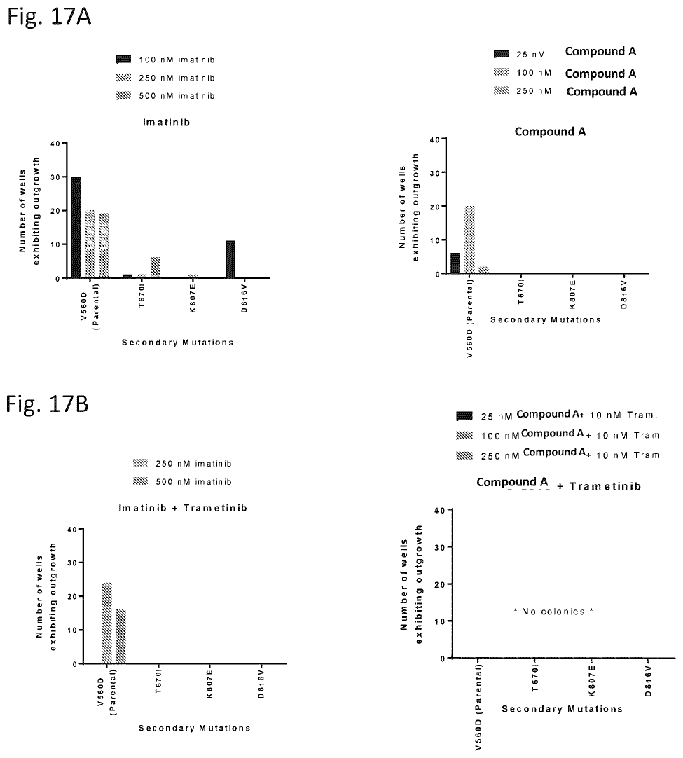

FIG. 17A is a graphical representation demonstrating the growth of Ba/F3 colonies resistant to imatinib. T670I, K807E, and/or D816V imatinib-resistant secondary KIT mutants were identified by PCR and DNA sequencing of genomic DNA in Ba/F3 cells exposed to 100 nM, 250 nM, or 500 nM imatinib as a single agent (FIG. 17A, left panel). FIG. 17A (right panel) is a graphical representation of the Ba/F3 cell saturation mutagenesis study with Compound A. Single agent treatment with 25 nM, 100 nM, or 250 nM Compound A did not lead to the outgrowth of any new secondary resistant mutation as determined by PCR and DNA sequencing. Only Ba/F3 cells containing the original V560D (parental) KIT mutation were shown to grow after exposure to Compound A, likely reflecting mutation in genes other than KIT (FIG. 17A, right panel). FIG. 17B is a graphical representation demonstrating the Ba/F3 cell colony outgrowth with imatinib in the presence of trametinib or Compound A in the presence of trametinib. Combination of imatinib at 250 nM or 500 nM with 10 nM trametinib did not lead to outgrowth of any new secondary resistant mutations but did lead to outgrowth of the original KIT V560D (parental) cells, likely reflecting mutation in genes other than KIT (FIG. 17B, left panel). Significantly, and in contrast to the combination study of imatinib with trametinib, combination of 25, 100, or 250 nM Compound A with 10 nM trametinib led to complete cell stasis or cell killing with no cell outgrowth to the limit of detection as determined by visual inspection in any wells (FIG. 17B, right panel).

### Example 33

### In vivo Xenograft Study of Compound A in Combination with Trametinib

The GIST T1 xenograft model was performed in compliance with all the laws, regulations and guidelines of the National Institutes of Health (NIH) and with the approval of the Animal Care and Use Committee of MI Bioresearch (Ann Arbor, MI), an AAALAC accredited facility. All mice had food and water ad libitum. All mice were observed for clinical signs at least once daily. Female Envigo nude mice (HsdCrl: Athymic Nude-NU-Foxn1nu; 6-7 weeks old) were inoculated subcutaneously just below the right high axilla with five million cells in Dulbecco's Phosphate Buffered Saline mixed with an equal volume of Matrigel, using a 27 gauge needle and syringe. When tumor burdens reached 117 mm3 on average on day 10, mice were randomly assigned into groups such that the mean tumor burden for all groups was within 10% of the overall mean tumor burden for the study population. Groups were treated on days 10-27 as follows: vehicle control diet (n=10); Compound A formulated into the mouse diet to achieve approximately 100 mg/kg/day of Compound A (n=10); or Compound A formulated into mouse diet to achieve approximately 25 mg/kg/day of Compound A (n=10), trametinib dosed orally at 0.5 mg/kg BID and fed vehicle control diet (n=10), trametinib dosed orally at 0.5 mg/kg BID and fed Compound A-formulated diet (achieving treatment with approximately 100 mg/kg/day of Compound A) (n=10), or trametinib dosed orally at 0.5 mg/kg BID and fed Compound A-formulated diet (achieving treatment with approximately 25 mg/kg/day of Compound A (n=10). On Day 27, all animals were placed on control diet to monitor tumor regrowth. Tumor volume and body weight were measured thrice weekly. Tumor burden (mg) was estimated from caliper measurements by the formula: tumor burden (mg=mm³)=(length×width²)/2.

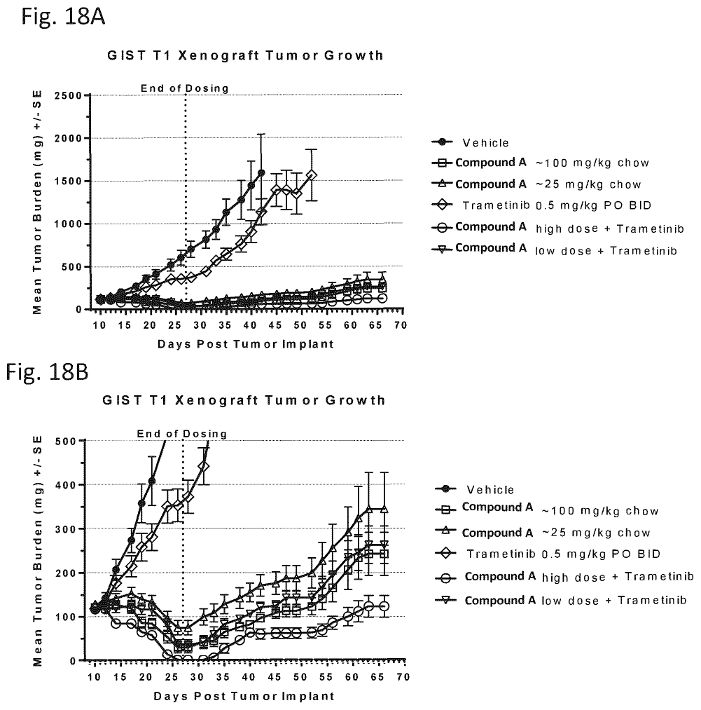

FIG. 18A and FIG. 18B are graphical representation demonstrating inhibition of tumor growth compared to vehicle control. FIG. 18B is the same data as FIG. 18A, but zoomed in to show differences among Compound A or Compound A/trametinib treated cohorts. Treatment with trametinib led to slight tumor growth inhibition compared to vehicle control. At the high dose of Compound A (approximately 100 mg/kg/day), 6/10 mice had complete tumor regression, with the remaining 4/10 mice having partial tumor regression during the dosing period. At the low dose of Compound A (approximately 25 mg/kg/day), 2/10 mice had complete tumor regression, and 6/10 had partial tumor regression. At the high dose of Compound A (approximately 100 mg/kg/day) combined with trametinib, 10/10 mice had complete tumor regression during the dosing period. At the low dose of Compound A (approximately 25 mg/kg/day) combined with trametinib, 5/10 mice had complete tumor regression, and 5/10 had partial tumor regression. In addition, after the dosing period, all Compound A treated cohorts had slower tumor regrowth than initial tumor growth of the vehicle control, indicating a prolonged effect on tumor cell growth through the end of the study on day 66. At the high dose of Compound A (approximately 100 mg/kg/day), 1/10 mice remained in partial tumor regression at the end of study. At the low dose of Compound A (approximately 25 mg/kg/day), 2/10 mice remained in partial tumor regression at the end of study. At the high dose of Compound A (approximately 100 mg/kg/day) combined with trametinib, 1/10 mice retained complete tumor regression and 4/10 mice remained in partial tumor regression at the end of the study. At the low dose of Compound A (approximately 25 mg/kg/day) combined with trametinib, 2/10 mice remained in partial tumor regression at the end of study. These data demonstrate that the combination of Compound A and trametinib induces cell death and/or prolonged cell stasis for at least 40 days after dosing was completed.

### Example 34

### Compound A is a Potent Inhibitor of the BCRP Efflux Transporter

To examine inhibition of the BCRP drug efflux transporter with Compound A, a vesicular transport inhibition assays was conducted using a low permeability probe substrate and inside-out membrane vesicles prepared from BCRP-expressing cells in the presence of ATP. The potential of Compound A to modify the uptake of the probe substrate into the transporter-containing vesicles was measured.

The in vitro interaction potential of Compound A with human efflux transporter BCRP was investigated at 7 concentrations in vesicular transport inhibition assays. Compound A potently inhibited the transport of the probe substrate of BCRP, where 44% inhibition was observed in the lowest concentration tested (0.04 μM). The IC50 value was estimated to be approximately 0.04 μM.

### Equivalents

Those skilled in the art will recognize, or be able to ascertain, using no more than routine experimentation, numerous equivalents to the specific embodiments described specifically in this disclosure. Such equivalents are intended to be encompassed in the scope of the following claims.

What is claimed is:

1. A method of treating a solid tumor in an imatinib resistant patient, comprising:
administering to the patient an effective amount of 1-[4-bromo-5-[1-ethyl-7-(methylamino)-2-oxo-1,2-dihydro-1,6-naphthyridin-3-yl]-2-fluorophenyl]-3- phenylurea, or a pharmaceutically acceptable salt thereof; and administering to the patient an effective amount of a MAPKAP kinase inhibitor selected from the group consisting of trametinib, binimetinib, cobimetinib, and ulixertinib, wherein the solid tumor is selected from the group consisting of of lung adenocarcinoma, squamous cell lung cancer, glioblastoma, pediatric glioma, astrocytoma, sarcoma, gastrointestinal stromal tumor (GIST), and melanoma.

2. The method of claim 1, further comprising administering a RAF inhibitor.

3. The method of claim 2, wherein the RAF inhibitor is a pan-RAF inhibitor.

4. A method of treating a solid tumor in a patient in need thereof, comprising
administering to the patient an effective amount of 1-[4-bromo-5-[1-ethyl-7-(methylamino)-2-oxo-1,2-dihydro-1,6-naphthyridin-3-yl]-2-fluorophenyl]-3- phenylurea, or a pharmaceutically acceptable salt thereof; and
administering to the patient an effective amount of a RAF inhibitor.

5. The method of claim 4, wherein the solid tumor is selected from the group consisting of lung adenocarcinoma, squamous cell lung cancer, GIST, and melanoma.

6. The method of claim 4, wherein the solid tumor has one or more mutations of the c-KIT gene.

7. The method of claim 4, wherein the RAF inhibitor is a pan-RAF inhibitor.

8. The method of claim 4, wherein the RAF inhibitor is dabrafenib or LY3009120.
